(19) United States
(12) Patent Application Publication
(10) Pub. No.: US 2012/0309750 A1
(43) Pub. Date: Dec. 6, 2012

(54) 5-MEMBERED HETEROCYCLE DERIVATIVES AND MANUFACTURING PROCESS THEREOF

(75) Inventors: Jae-Hoon Kang, Seoul (KR); Hong-Sub Lee, Yongin-si (KR); Jin-Sun Kwon, Seongnam-si (KR); Joon-Tae Park, Yongin-si (KR); Chang-Sung Hong, Yongin-si (KR); Dong-Hyung Sin, Hwaseong-si (KR); Soo-Jung Hong, Hwaseong-si (KR); An-Na Moon, Hwaseong-si (KR); Jin-Ah Jeong, Seoul (KR); Sung-Wook Kwon, Gyeonggi-do (KR)

(73) Assignee: ILDONG PHARM CO., LTD., Seoul (KR)

(21) Appl. No.: 13/579,507

(22) PCT Filed: Feb. 17, 2011
(86) PCT No.: PCT/KR11/01068

§ 371 Date: Aug. 16, 2012

(30) Foreign Application Priority Data
Feb. 17, 2010 (KR) 10-2010-0014173
Feb. 17, 2011 (KR) 10-2011-0013917

(51) Int. Cl. C07D 413/14; C07D 413/04; A61P 35/00; C07D 417/14; A61K 31/541; A61K 31/5377; A61K 31/422

(52) U.S. Cl. 514/227.8; 544/137; 514/236.8; 548/247; 514/378; 544/58.7; 544/58.4

(57) ABSTRACT

Disclosed are a novel 5-membered heterocycle derivatives of Formula I, a tautomer, pharmaceutically acceptable salt, prodrug thereof and a pharmaceutical use thereof. The 5-membered heterocycle derivatives of Formula I, a tautomer, pharmaceutically acceptable salt, prodrug thereof and compositions containing them are useful for treating a tumor.

### TECHNICAL FIELD

This present invention relates to a novel 5-membered heterocycle derivatives, a tautomer, a pharmacologically acceptable salt, prodrug or pharmaceutical use thereof.

### BACKGROUND ART

Molecular chaperones are a general term for proteins that form a complex temporally with client proteins to promote the formation of the conformation of the client proteins. These proteins, the activity of which is to help folding and association of protein and to prevent aggregation are broadly defined as molecular chaperones.

Exposure of cells to a number of environmental stresses, including heat shock, alcohol, heavy metals and oxidative stress, results in the cellular accumulation of a number of chaperones, commonly known as heat shock proteins(HSPs). Molecular chaperones of the “heat shock proteins” family (HSPs), classified according to their molecular mass (HSP70, HSP90, HSP27, etc.), protect the cell against the initial stress insult, enhances recovery and leads to maintenance of a stress tolerant state. It has also become clear, however, that certain HSPs may also play a major molecular chaperone role under normal, stress-free conditions by regulating the correct folding, degradation, localization and function of a growing list of important cellular proteins.

Several diseases in humans can be acquired as a result of protein misfolding. In some conditions (e.g., Alzheimer's disease, prion diseases and Huntington's disease), misfolded proteins can cause protein aggregation resulting in neurodegenerative disorders ([Tytell M. and Hooper P. L., Emerging Ther. Targets (2001), 5, 3788-3796]). HSPs, and in particular HSP90, are also involved in the regulation of various major functions of the tumor cell, via their association with various client proteins involved in cell proliferation or apoptosis. In these pathologies, approaches aimed at breaking up or at disturbing the function of chaperones could be available for treatment of disease. Especially, HSP90 chaperons has recently been demonstrated as a particularly promising target in anticancer therapy([Moloney A. and Workman P., Expert Opin. Biol. Ther. (2002), 2(1), 3-24]; [Choisis et al, Drug Discovery Today (2004), 9, 881-888]).

HSP90 (Heat Shock Protein 90) family proteins included HSP90α, HSP90β, GRP94 and HSP75/TRAP1. These proteins represent approximately 1-2% of the total cellular protein mass. It is usually in the form of a dimer in the cell and is associated with multiplicity of proteins, so-called co-chaperones. HSP90 plays a key role in the response to cellular stress by interaction with many proteins whose native folding has been modified by external stress, such as, for example, heat shock, in order to restore the original folding or to prevent aggregation of the proteins ([Smith D. F. et al., Pharmacological Rev. (1998), 50, 493-513]). There are also indications that HSP90 is of importance as buffer against the effects of mutations, presumably through correction of incorrect protein folding caused by the mutation([Rutherford and Lindquist, 1998]). HSP90 also has a regulatory importance. Under physiological conditions, HSP90, together with its homologue in the endoplasmatic reticulum, GRP94, plays a role in the cell balance for ensuring the stability of the conformation and maturing of various client key proteins, such as, EGFR R/HER2, Src, Akt, Raf, MEK, Bcr-Abl, Flt-3, mutated p53, Akt, survivin, Cdk4, Plk, Weel, VEGF-R, FAK, HIF-1, hTert and c-Met, etc. These client proteins are involved in the six mechanisms of tumour progression. i) An ability to proliferate in the absence of growth factor(EGFR-R/HER2, Src, Akt, Raf, MEK, Bcr-Abl, Flt-3, etc.); ii) An ability to evade apoptosis (mutated form of p53, Akt, survivin, etc.); iii) An insensitivity to proliferation stop signal(Cdk4, Plk, Weel, etc.); iv) An ability to activate angiogenesis(VEGF-R, FAK, HIF-1, Akt, etc.); v) An ability to proliferate with no replicative limit (hTert, etc.); vi) An ability to evade new tissue and to metastasize(c-Met); (Hanahan D. and Weinberg R. A., Cell (2002), 100, 57-70). Therefore, the client protein-induced tumor formation can be inhibited by inhibition of HSP90 activity.

The first known HSP90 inhibitors are compounds of the ansamycin family, in particular geldanamycin and herbimycin A. X-ray studied have shown that geldanamycin binds to the ATP site of the N-terminal domain of HSP90, Where it inhibits the ATPase activity of the chaperone(Prodromou C. et al, Cell (1997), 90, 65-75). Currently, the NIH and Kosan BioScience are carrying out the clinical development of 17AAG, which is a geldanamycin-derived HSP90 inhibitor.

Radicicol is also an Hsp90 inhibitor of natural origin ([Roe S. M. et al, J. Med. Chem. (1999), 42, 260-66]). However, although the latter is by far the best in vitro Hsp90 inhibitor, its metabolic instability with respect to sulphur-containing nucleophiles makes it difficult to use in vivo. One Hsp90 inhibitor of natural origin, novobiocin, binds to a different ATP site located in the C-terminal domain of the protein ([Itoh H. et al, Biochem J. (1999), 343, 697-703]). Purines, such as the compound PU3 ([Chiosis et al, Chem. Biol. (2001), 8, 289-299]), have also been described as small molecule Hsp90 inhibitors.

In addition, the analogues, such as 8-heteroaryl-6-phenylimidazo[1,2-a]pyrazines(WO 2004/072080), pyrazoles derivatives (WO 2004/050087), isoxazole derivatives (WO 2004/07051) and benzophonone derivatives (WO 2005/00778) have also been described as HSP90 inhibitor, that are useful for the treatment of tumors.

The known HSP90 inhibitors involve binding to HSP90 at the ATP binding site located in the N-terminal domain of the protein, leading to inhibition of the intrinsic ATPase activity of HSP90. Inhibition of HSP90 ATPase activity prevents recruitment of co-chaperons, which these client proteins are targeted for degradation via the ubiquitin proteasome pathway. An attractive rationale for developing drugs against this target for use in the clinic is that by simultaneously depleting tumor and associated with the client proteins, one may obtain a strong antitumor effect and achieve a therapeutic advantage against cancer versus normal cells.

### Disclosure

### Technical Problem

Accordingly, the present invention is designed to provide a novel compound having a superior HSP90 inhibitory activity for prevention and treatment cancer.

It is another object of the prevent invention to provide a pharmaceutical composition containing the novel compound having a superior HSP90 inhibitory activity as an active ingredient for prevention and treatment cancer.

### Technical Solution

To solve the problem described above, the present invention shows the novel compound, a 5-membered heterocycle derivative represented by the following general Formula I.

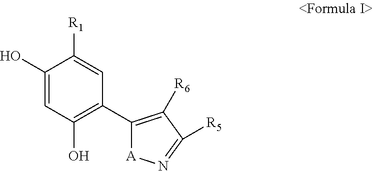

wherein,
A represents a nitrogen atom or oxygen atom;
R₁ represents chloro or isopropyl;
R₅ represents CH₂R_{d} or N-ethylcarboxamide (especially, R_{d} represents hydroxy, acetamido, propionamido or triazolyl);
R₆ represents

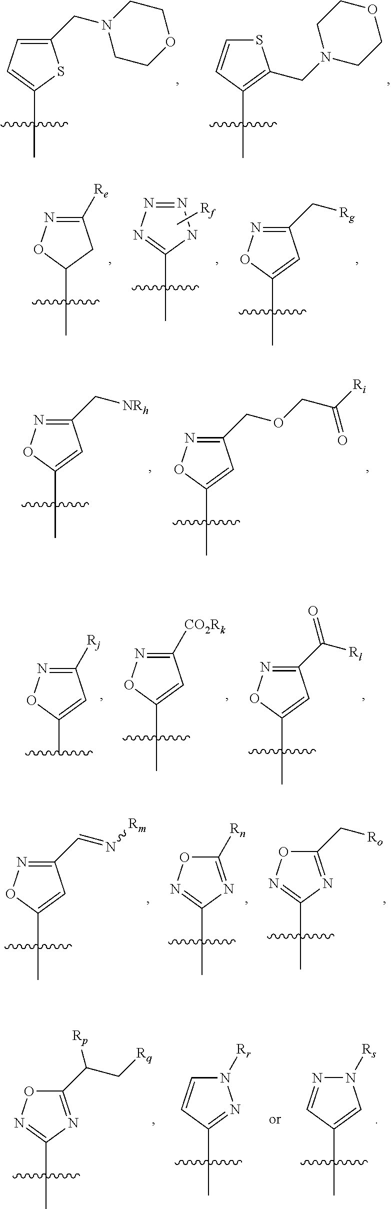

especially,
Rₑ represents hydroxymethyl, ethylcarboxylate or N-ethylcarboxamide;
R_{f} represents hydrogen, methyl or ethyl;
R_{g} represents hydrogen, hydroxy, fluoro, cyano, ethylamino, hydroxyethylamino, dimethylamino, diethylamino, isopropylamino, allylamino, diisopropylamino, piperidinyl, pyrrolidinyl, 4-methylpiperazinyl, morpholino, thiomorpholino or methanesulfonyl;
Rₕ represents hydrogen, acetyl or propionyl;
Rᵢ represents hydroxy, methoxy or amino;
Rⱼ, represents cyano, thiophenyl, phenyl or dimethoxymethyl;
Rₖ represents hydrogen or ethyl;
Rₗ represents amino, methylamino, ethylamino, morpholino or thiomorpholino;
Rₘ represents hydroxy, methoxy, ethoxy or allyloxy;
Rₙ represents hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl;
Rₒ represents hydroxy, morpholino, dimethylamino, piperidinyl or pyrrolidinyl;
Rₚ represents (S)-hydroxy or hydroxy;
R_{q} represents hydrogen or chloro;
Rᵣ represents hydrogen, methyl, ethyl, isopropyl or n-propyl;
Rₛ represents hydrogen, methyl, ethyl, or isopropyl.

In the present invention, the desired compound of Formula I is selected from i) or x) disclosed below.

1) A is oxygen, R₁ is isopropyl, R₅ is N-ethylcarboxamide, R₆ is

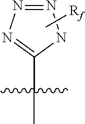

(especially, R_{f} is hydrogen, methyl or ethyl).

2) A is oxygen, R₁ is chloro or isopropyl, R₅ is N-ethylcarboxamide, R₆

is

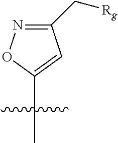

(especially, R_{g} is hydrogen, hydroxy, fluoro, cyano, ethylamino, hydroxyethylamino, dimethylamino, diethylamino, isopropylamino, allylamino, diisopropylamino, piperidinyl, pyrrolidinyl, 4-methylpiperazinyl, morpholino, thiomorpholino or methanesulfonyl.

3) A is oxygen, R₁ is chloro or isopropyl, R₅ is N-ethylcarboxamide, R₆ is

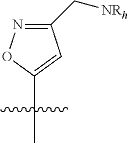

(especially, hydrogen, acetyl or propionyl).

4) A is nitrogen or oxygen, R₁ is isopropyl, R₅ is N-ethylcarboxamide, R₆ is

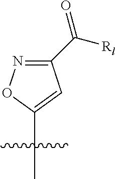

(especially, R₁ is amino, methylamino, ethylamino, morpholino or thiomorpholino).

5) A is oxygen, R₁ is isopropyl, R₅ is N-ethylcarboxamide, R₆ is

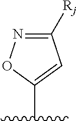

(especially, Rⱼ is dimethoxymethyl).

6) A is oxygen, R₁ is isopropyl, R₅ is N-ethylcarboxamide, R₆ is

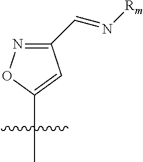

(especially, Rₘ is hydroxy, methoxy, ethoxy or allyoxy).

7) A is oxygen, R₁ is isopropyl, R₅ is N-ethylcarboxamide, R₆ is

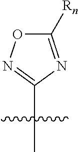

(especially, Rₙ is hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl).

8) A is oxygen, R₁ is isopropyl, R₅ is N-ethylcarboxamide, R₆ is

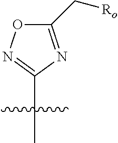

(especially, Rᵣ is hydrogen, methyl, ethyl, isopropyl or n-propyl).

9) A is oxygen, R₁ is isopropyl, R₅ is N-ethylcarboxamide, R₆ is

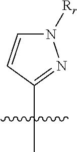

(especially, Rᵣ is hydrogen, methyl, ethyl, isopropyl or n-propyl).

10) A is oxygen, R₁ is isopropyl, R₅ is N-ethylcarboxamide, R₆ is

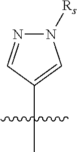

(especially, Rₛ is hydrogen, methyl, ethyl, or isopropyl).

Particularly preferred examples of the compound of Formula I according to the present invention include the following.
5-(2,4-Dihydroxy-5-isopropylphenyl)-N-ethyl-4-(5-(morpholinomethyl)thiophen-2-yl)isoxazole-3-carboxamide; (I-1)
5-(2,4-Dihydroxy-5-isopropylphenyl)-N-ethyl-4-(2-(morpholinomethyl)thiophen-3-yl)isoxazole-3-carboxamide; (I-2)
4-(3-(Hydroxymethyl)-4-(thiophen-3-yl)isoxazol-5-yl)-6-isopropylbenzene-1,3-d iol; (I-3)
N-((5-(2,4-dihydroxy-5-isopropylphenyl)-4-(thiophen-3-yl)isoxazol-3-yl)methyl) propionamide; (I-4)
4-((3-((1H-1,2,3-triazol-1-yl)methyl)-4(thiophen-3-yl)isoxazol-5-yl)-6-isopro pylbenzene-1,3-diol; (I-5)
N-((5-(2,4-dihydroxy-5-isopropylphenyl)-4-(thiophen-3-yl)isoxazol-3-yl)methyl)acetamide; (I-6)
Ethyl 5-(5-(2,4-dihydroxy-5-isopropylphenyl)-3-(ethylcarbamoyl)isoxazol-4-yl)-4,5-dihydroisoxazole-3-carboxylate; (I-7)
5-(2,4-Dihydroxy-5-isopropylphenyl)-N-ethyl-4-(3-(ethylcarbamoyl)-4,5-dihydro isoxazol-5-yl)isoxazole-3-carboxamide; (I-8)
5-(2,4-Dihydroxy-5-isopropylphenyl)-N-ethyl-4-(3-(hydroxymethyl)-4,5-dihydroi soxazol-5-yl)isoxazole-3-carboxamide; (I-9)
5-(2,4-Dihydroxy-5-isopropylphenyl)-N-ethyl-4-(1H-tetrazol-5-yl)isoxazole-3-carboxamide; (I-10)
5-(2,4-Dihydroxy-5-isopropylphenyl)-N-ethyl-4-(1-methyl-1H-tetrazol-5-yl)isox azole-3-carboxamide; (I-11)
5-(2,4-Dihydroxy-5-isopropylphenyl)-N-ethyl-4-(2-methyl-2H-tetrazol-5-yl)isox azole-3-carboxamide; (I-12)
5-(2,4-Dihydroxy-5-isopropylphenyl)-N-ethyl-4-(1-ethyl-1H-tetrazol-5-yl)isoxa zole-3-carboxamide; (I-13)
5-(2,4-Dihydroxy-5-isopropylphenyl)-N-ethyl-4-(2-ethyl-2H-tetrazol-5-yl)isoxa zole-3-carboxamide; (I-14)
Ethyl 5-(2,4-dihydroxy-5-isopropylphenyl)-3-(ethylcarbamoyl)-4,5′-biisoxazole-3′-carboxylate; (I-15)
5-(2,4-Dihydroxy-5-isopropylphenyl)-N³,N³′-diethyl-4,5′-biisoxazole-3,3′-dicarb oxamide; (I-16)
5-(2,4-Dihydroxy-5-isopropylphenyl)-N-ethyl-3′-(hydroxymethyl)-4,5′-biisoxazo le-3-carboxamide; (I-17)
5-(2,4-Dihydroxy-5-isopropylphenyl)-N-ethyl-3′-(morpholinomethyl)-4,5′-biisox azole-3-carboxamide; (I-18)
Methyl 2-((5-(2,4-dihydroxy-5-isopropylphenyl)-3-(ethylcarbamoyl)-4,5′-biisoxazol-3′-yl)methoxy)acetate; (I-19)
3′-((Diethylamino)methyl)-5-(2,4-dihydroxy-5isopropylphenyl)-N-ethyl-4,5′-bi isoxazole-3-carboxamide; (I-20)
5-(2,4-Dihydroxy-5-isopropylphenyl)-N³-ethyl-4,5′-biisoxazole-3,3′-dicarboxami de; (I-21)
5-(2,4-Dihydroxy-5-isopropylphenyl)-N-ethyl-3′-((2-hydroxyethylamino)methyl)-4,5′-biisoxazole-3-carboxamide; (I-22)
5-(2,4-Dihydroxy-5-isopropylphenyl)-N-ethyl-3′-(morpholine-4-carbonyl)-4,5′-b iisoxazole-3-carboxamide; (I-23)
5-(5-Chloro-2,4-dihydroxyphenyl)-N-ethyl-3′-(hydroxymethyl)-4,5′-biisoxazole-3-carboxamide; (I-24)
Ethyl 5-(5-chloro-2,4-dihydroxyphenyl)-3-(ethylcarbamoyl)-4,5′-biisoxazole-3′-carboxylate; (I-25)
5-(2,4-dihydroxy-5-isopropylphenyl)-3-(ethylcarbamoyl)-4,5′-biisoxazole-3′-carboxylic acid; (I-26)
5-(2,4-Dihydroxy-5-isopropylphenyl)-N-ethyl-3′-((ethylamino)methyl)-4,5′-biisoxazole-3-carboxamide; (I-27)
5-(2,4-Dihydroxy-5-isopropylphenyl)-N-ethyl-3′-(fluoromethyl)-4,5′-biisoxazole-3-carboxamide; (I-28)
5-(5-(2,4-Dihydroxy-5-isopropylphenyl)-3-(ethylcarbamoyl)-1H-pyrazol-4-yl)-N-ethylisoxazole-3-carboxamide; (I-29)
3′-(Aminomethyl)-5-(2,4-dihydroxy-5-isopropylphenyl)-N-ethyl-4,5′-biisoxazole-3-carboxamide; (I-30)
3′-(Acetamidomethyl)-5-(2,4-dihydroxy-5-isopropylphenyl)-N-ethyl-4,5′-biisoxa zole-3-carboxamide; (I-31)
(5-(2,4-Dihydroxy-5-isopropylphenyl)-3-(ethylcarbamoyl)-4,5′-biisoxazol-3′-yl)methyl methanesulfonate; (I-32)
2-((5-(2,4-Dihydroxy-5-isopropylphenyl)-3-(ethylcarbamoyl)-4,5′-biisoxazol-3′-yl)methoxy)acetic acid; (I-33)
5-(2,4-Dihydroxy-5-isopropylphenyl)-N-ethyl-3′-(propionamidomethyl)-4,5′-biis oxazole-3-carboxamide; (I-34)
3′-(Cyanomethyl)-5-(2,4-dihydroxy-5-isopropylphenyl)-N-ethyl-4,5′-biisoxazole-3-carboxamide; (I-35)
3′-((2-Amino-2-oxoethoxy)methyl)-5-(2,4-dihydroxy-5-isopropylphenyl)-N-ethyl-4,5′-biisoxazole-3-carboxamide; (I-36)
5-(2,4-Dihydroxy-5-isopropylphenyl)-N-ethyl-3′-methyl-4,5′-biisoxazole-3-carb oxamide; (I-37)
5-(2,4-Dihydroxy-5-isopropylphenyl)-N-ethyl-3′-(piperidin-1-ylmethyl)-4,5′-bi isoxazole-3-carboxamide; (I-38)
5-(2,4-Dihydroxy-5-isopropylphenyl)-N-ethyl-3′-(pyrrolidin-1-ylmethyl)-4,5′-b iisoxazole-3-carboxamide; (I-39)
5-(2,4-Dihydroxy-5-isopropylphenyl)-N-ethyl-3′-((isopropylamino)methyl)-4,5′-biisoxazole-3-carboxamide; (I-40)
5-(2,4-Dihydroxy-5-isopropylphenyl)-3′-((dimethylamino)methyl)-N-ethyl-4,5′-b iisoxazole-3-carboxamide; (I-41)
5-(2,4-Dihydroxy-5-isopropylphenyl)-N-ethyl-3′-((methylamino)methyl)-4,5′-bii soxazole-3-carboxamide; (I-42)
5-(2,4-Dihydroxy-5-isopropylphenyl)-N-ethyl-3′-((4-methylpiperazin-1-yl)methyl)-4,5′-biisoxazole-3-carboxamide; (I-43)
3′-((Allylamino)methyl)-5-(2,4-dihydroxy-5-isopropylphenyl)-N-ethyl-4,5′-biis oxazole-3-carboxamide; (I-44)
5-(2,4-Dihydroxy-5-isopropylphenyl)-3′-((dipropylamino)methyl)-N-ethyl-4,5′-b iisoxazole-3-carboxamide; (I-45)
5-(2,4-Dihydroxy-5-isopropylphenyl)-N-ethyl-3′-(thiophen-3-yl)-4,5′-biisoxazole-3-carboxamide; (I-46)
5-(2,4-Dihydroxy-5-isopropylphenyl)-N-ethyl-3′-(thiomorpholinomethyl)-4,5′-biisoxazole-3-carboxamide; (I-47)
3′-Cyano-5-(2,4-dihydroxy-5-isopropylphenyl)-N-ethyl-4,5′-biisoxazole-3-carboxamide; (I-48)
5-(2,4-Dihydroxy-5-isopropylphenyl)-N-ethyl-3′-phenyl-4,5′-biisoxazole-3-carboxamide; (I-49)
5-(2,4-Dihydroxy-5-isopropylphenyl)-N-ethyl-3′-(thiomorpholine-4-carbonyl)-4,5′-biisoxazole-3-carboxamide; (I-50)
5-(2,4-Dihydroxy-5-isopropylphenyl)-3′-(dimethoxymethyl)-N-ethyl-4,5′-biisoxa zole-3-carboxamide; (I-51)
5-(2,4-Dihydroxy-5-isopropylphenyl)-N-ethyl-3′-((methoxyimino)methyl)-4,5′-bi isoxazole-3-carboxamide; (I-52)
5-(2,4-Dihydroxy-5-isopropylphenyl)-N-ethyl-3′-((hydroxyimino)methyl)-4,5′-bi isoxazole-3-carboxamide; (I-53)
3′-((Allyloxyimino)methyl)-5-(2,4-dihydroxy-5-isopropylphenyl)-N-ethyl-4,5′-b iisoxazole-3-carboxamide; (I-54)
5-(2,4-Dihydroxy-5-isopropylphenyl)-3′-((ethoxyimino)methyl)-N-ethyl-4,5′-bii soxazole-3-carboxamide; (I-55)
5-(2,4-Dihydroxy-5-isopropylphenyl)-N³-ethyl-N^{3′}-methyl-4,5′-biisoxazole-3,3′-d icarboxamide; (I-56)
5-(2,4-Dihydroxy-5-isopropylphenyl)-N-ethyl-4-(5-(trifluoromethyl)-1,2,4-oxad iazol-3-yl)isoxazole-3-carboxamide; (I-57)
Methyl 3-(5-(2,4-dihydroxy-5-isopropylphenyl)-3-(ethylcarbamoyl)isoxazol-4-yl)-1,2,4-oxadiazole-5-carboxylate; (I-58)
3-(5-(2,4-Dihydroxy-5-isopropylphenyl)-3-(ethylcarbamoyl)isoxazol-4-yl)-N-eth yl-1,2,4-oxadiazole-5-carboxamide; (I-59)
5-(2,4-Dihydroxy-5-isopropylphenyl)-N-ethyl-4-(5-methyl-1,2,4-oxadiazol-3-yl) isoxazole-3-carboxamide; (I-60)
5-(2,4-Dihydroxy-5-isopropylphenyl)-N-ethyl-4-(5-phenyl-1,2,4-oxadiazol-3-yl) isoxazole-3-carboxamide; (I-61)
5-(2,4-Dihydroxy-5-isopropylphenyl)-N-ethyl-4-(5-ethyl-1,2,4-oxadiazol-3-yl)isoxazole-3-carboxamide; (I-62)
5-(2,4-Dihydroxy-5-isopropylphenyl)-N-ethyl-4-(5-(furan-2-yl)-1,2,4-oxadiazol-3-yl)isoxazole-3-carboxamide; (I-63)
3-(5-(2,4-Dihydroxy-5-isopropylphenyl)-3-(ethylcarbamoyl)isoxazol-4-yl)-N,N-dimethyl-1,2,4-oxadiazole-5-carboxamide; (I-64)
5-(2,4-Dihydroxy-5-isopropylphenyl)-N-ethyl-4-(5-isopropyl-1,2,4-oxadiazol-3-yl)isoxazole-3-carboxamide; (I-65)
5-(2,4-Dihydroxy-5-isopropylphenyl)-N-ethyl-4-(1,2,4-oxadiazol-3-yl)isoxazole-3-carboxamide; (I-66)
5-(2,4-Dihydroxy-5-isopropylphenyl)-N-ethyl-4-(5-(morpholine-4-carbonyl)-1,2 4-oxadiazol-3-yl)isoxazole-3-carboxamide; (I-67)
5-(2,4-Dihydroxy-5-isopropylphenyl)-N-ethyl-4-(5-(pyrrolidine-1-carbonyl)-1,2,4-oxadiazol-3-yl)isoxazole-3-carboxamide; (I-68)
5-(2,4-Dihydroxy-5-isopropylphenyl)-N-ethyl-4-(5-(pyrrolidin-1-yl)-1,2,4-oxadiazol-3-yl)isoxazole-3-carboxamide; (I-69)
5-(2,4-Dihydroxy-5-isopropylphenyl)-N-ethyl-4-(5-(trichloromethyl)-1,2,4-oxadiazol-3-yl)isoxazole-3-carboxamide; (I-70)
5-(2,4-Dihydroxy-5-isopropylphenyl)-N-ethyl-4-(5-(piperidin-1-yl)-1,2,4-oxadiazol-3-yl)isoxazole-3-carboxamide; (I-71)
5-(2,4-Dihydroxy-5-isopropylphenyl)-4-(5-(dimethylamino)-1,2,4-oxadiazol-3-yl)-N-ethylisoxazole-3-carboxamide; (I-72)
5-(2,4-Dihydroxy-5-isopropylphenyl)-N-ethyl-4-(5-morpholino-1,2,4-oxadiazol-3-yl)isoxazole-3-carboxamide; (I-73)
3-(5-(2,4-Dihydroxy-5-isopropylphenyl)-3-(ethylcarbamoyl)isoxazol-4-yl)-N,N-diethyl-1,2,4-oxadiazole-5-carboxamide; (I-74)
5-(2,4-Dihydroxy-5-isopropylphenyl)-N-ethyl-4-(5-(methoxymethyl)-1,2,4-oxadiazol-3-yl)isoxazole-3-carboxamide; (I-75)
4-(5-(Diethylamino)-1,2,4-oxadiazol-3-yl)-5-(2,4-dihydroxy-5-isopropylphenyl)-N-ethylisoxazole-3-carboxamide; (I-76)
5-(2,4-Dihydroxy-5-isopropylphenyl)-N-ethyl-4-(5-(thiophen-2-yl)-1,2,4-oxadiazol-3-yl)isoxazole-3-carboxamide; (I-77)
4-(5-Amino-1,2,4-oxadiazol-3-yl)-5-(2,4-dihydroxy-5-isopropylphenyl)-N-ethylisoxazole-3-carboxamide; (I-78)
5-(2,4-Dihydroxy-5-isopropylphenyl)-N-ethyl-4-(5-(methylamino)-1,2,4-oxadiazol-3-yl)isoxazole-3-carboxamide; (I-79)
5-(2,4-Dihydroxy-5-isopropylphenyl)-N-ethyl-4-(5-hydroxy-1,2,4-oxadiazol-3-yl) isoxazole-3-carboxamide; (I-80)
5-(2,4-Dihydroxy-5-isopropylphenyl)-N-ethyl-4-(5-(hydroxymethyl)-1,2,4-oxadiazol-3-yl)isoxazole-3-carboxamide; (I-81)
5-(2,4-Dihydroxy-5-isopropylphenyl)-N-ethyl-4-(5-mercapto-1,2,4-oxadiazol-3-yl)isoxazole-3-carboxamide; (I-82)
(S)-5-(2,4-Dihydroxy-5-isopropylphenyl)-N-ethyl-4-(5-(1-hydroxyethyl)-1,2,4-oxadiazol-3-yl)isoxazole-3-carboxamide; (I-83)
5-(2,4-Dihydroxy-5-isopropylphenyl)-N-ethyl-4-(5-(4-methoxyphenyl)-1,2,4-oxadiazol-3-yl)isoxazole-3-carboxamide; (I-84)
5-(2,4-Dihydroxy-5-isopropylphenyl)-N-ethyl-4-(5-(4-nitrophenyl)-1,2,4-oxadiazol-3-yl)isoxazole-3-carboxamide; (I-85)
5-(2,4-Dihydroxy-5-isopropylphenyl)-N-ethyl-4-(5-methoxy-1,2,4-oxadiazol-3-yl) isoxazole-3-carboxamide; (I-86)
5-(2,4-Dihydroxy-5-isopropylphenyl)-N-ethyl-4-(5-(methylthio)-1,2,4-oxadiazol-3-yl)isoxazole-3-carboxamide; (I-87)
4-(5-Cyclopentyl-1,2,4-oxadiazol-3-yl)-5-(2,4-dihydroxy-5-isopropylphenyl)-N-ethylisoxazole-3-carboxamide; (I-88)
4-(5-Cyclohexyl-1,2,4-oxadiazol-3-yl)-5-(2,4-dihydroxy-5-isopropylphenyl)-N-e thylisoxazole-3-carboxamide; (I-89)
5-(2,4-Dihydroxy-5-isopropylphenyl)-4-(5-(4-ethoxyphenyl)-1,2,4-oxadiazol-3-yl)-N-ethylisoxazole-3-carboxamide; (I-90)
4-(5-(2-Chloro-1-hydroxyethyl)-1,2,4-oxadiazol-3-yl)-5-(2,4-dihydroxy-5-isopr opylphenyl)-N-ethylisoxazole-3-carboxamide; (I-91)
5-(2,4-Dihydroxy-5-isopropylphenyl)-N-ethyl-4-(5-(morpholinomethyl)-1,2,4-oxa diazol-3-yl)isoxazole-3-carboxamide; (I-92)
5-(2,4-Dihydroxy-5-isopropylphenyl)-4-(5-((dimethylamino)methyl)-1,2,4-oxadia zol-3-yl)-N-ethylisoxazole-3-carboxamide; (I-93)
5-(2,4-Dihydroxy-5-isopropylphenyl)-N-ethyl-4-(5-(piperidin-1-ylmethyl)-1,2,4-oxadiazol-3-yl)isoxazole-3-carboxamide; (I-94)
5-(2,4-Dihydroxy-5-isopropylphenyl)-N-ethyl-4-(5-(pyrrolidin-1-ylmethyl)-1,2,4-oxadiazol-3-yl)isoxazole-3-carboxamide; (I-95)
5-(2,4-Dihydroxy-5-isopropylphenyl)-N-ethyl-4-(1H-pyrazol-3-yl)isoxazole-3-carboxamide; (I-96)
5-(2,4-Dihydroxy-5-isopropylphenyl)-N-ethyl-4-(1-methyl-1H-pyrazol-3-yl)isoxazole-3-carboxamide; (I-97)
5-(2,4-Dihydroxy-5-isopropylphenyl)-N-ethyl-4-(1-methyl-1H-pyrazol-5-yl)isoxazole-3-carboxamide; (I-98)
5-(2,4-Dihydroxy-5-isopropylphenyl)-N-ethyl-4-(1-ethyl-1H-pyrazol-3-yl)isoxazole-3-carboxamide; (I-99)
5-(2,4-Dihydroxy-5-isopropylphenyl)-N-ethyl-4-(1-isopropyl-1H-pyrazol-3-yl)isoxazole-3-carboxamide; (I-100)
5-(2,4-Dihydroxy-5-isopropylphenyl)-N-ethyl-4-(1-propyl-1H-pyrazol-3-yl)isoxazole-3-carboxamide; (I-101)
5-(2,4-Dihydroxy-5-isopropylphenyl)-N-ethyl-4-(1-methyl-1H-pyrazol-4-yl)isoxazole-3-carboxamide; (I-102)
5-(2,4-Dihydroxy-5-isopropylphenyl)-N-ethyl-4-(1H-pyrazol-4-yl)isoxazole-3-carboxamide; (I-103)
5-(2,4-Dihydroxy-5-isopropylphenyl)-N-ethyl-4-(1-ethyl-1H-pyrazol-4-yl)isoxazole-3-carboxamide; (I-104)
5-(2,4-Dihydroxy-5-isopropylphenyl)-N-ethyl-4-(1-isopropyl-1H-pyrazol-4-yl)isoxazole-3-carboxamide; (I-105)
Sodium 4-(3-(ethylcarbamoyl)-4-(5-methyl-1,2,4-oxadiazol-3-yl)isoxazol-5-yl)-6-isopropylbenzene-1,3-bis(olate); (I-106)

The derivative represented by Formula I, as used herein the pharmaceutically approved salts include base addition, acid addition and quaternary salts. Compounds of the present invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxylmethyl)aminomethane, L-arginine, L-lysine, N-ethylpiperidine, dibenzylamine and the like. The compounds of Formula I which are basic can form salts, including pharmaceutically acceptable salts with inorganic acids, e.g. with hydrohalic acids such as hydrorchloric or hydrobromic acids, sulfuric acid, nitric acid or phosphoric acid and the like, and with organic acids e.g. with acetic, tartaric, succinic, fumaric, maleic, malic, salicyclic, citric, methanesulfonic, p-toluenesulfonic, benzoic, benzenesulfonic, glutamic, lactic, and mandelic acids and the like.

Some compounds of the present invention contain one or more actual or potential chiral centres because of the presence of asymmetric carbon atoms. The presence of several asymmetric carbon atoms gives rise to a number of diastereoisomers with R or S stereochemistry at each chiral centre. Therefore, the present invention includes all such diasteroisomers and mixtures thereof.

In another aspect, the present invention provides a method of preparing the compound represented by Formula I or a pharmaceutically approved salt thereof.

A preparation method of the present invention is shown in the following

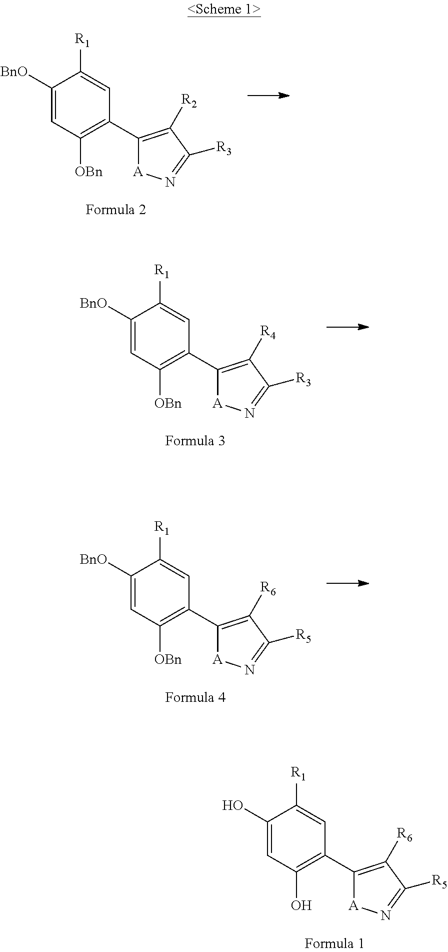

The compound of Formula I of the present invention, as shown in Scheme 1, can be prepared by a series of steps from the compound of Formula 2. A, R₁, R₂, R₃, R₄, R₅ and R₆, illustrated in Scheme 1, are the same as defined in Formula I and Formula 2˜Formula 4.

wherein,

A represents a nitrogen atom or an oxygen atom,

R₁ represents chloro or isopropyl;

R₂ represents iodo;

R₃ represents ethylcarboxylate or N-ethylcarboxamide;

R₄ represents cyano,

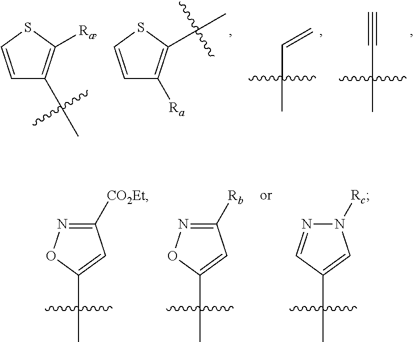

Especially, Rₐ represents hydrogen or formyl; R_{b} represents methyl, thiophenyl or phenyl; R_{c} represents hydrogen, trityl, methyl, ethyl or isopropyl;

R₅ represents CH₂R_{d} or N-ethylcarboxamide; Especially, R_{d} represents hydroxyl, acetamido, propionamido or triazolyl;

R₆ represents

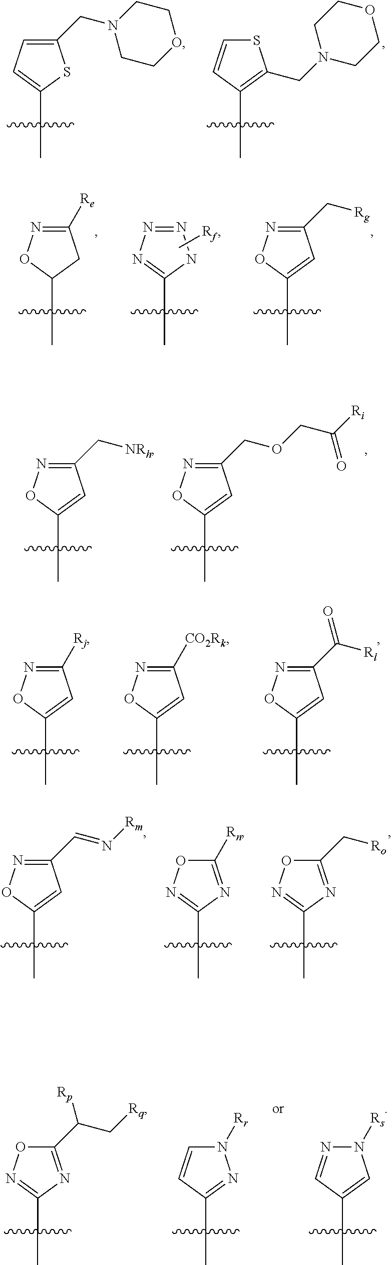

especially,

Rₑ represents hydroxymethyl, ethylcarboxylate or N-ethylcarboxamide;

R_{f} represents hydrogen, methyl or ethyl;

R_{g} represents hydrogen, hydroxy, fluoro, cyano, ethylamino, hydroxyethylamino, dimethylamino, diethylamino, isopropylamino, allylamino, diisopropylamino, piperidinyl, pyrrolidinyl, 4-methylpiperazinyl, morpholino, thiomorpholino or methanesulfonyl;

Rₕ represents hydrogen, acetyl or propionyl;

Rᵢ represents hydroxy, methoxy or amino;

Rⱼ represents cyano, thiophenyl, phenyl or dimethoxymethyl;

Rₖ represents hydrogen or ethyl;

Rₗ represents amino, methylamino, ethylamino, morpholino or thiomorpholino;

Rₘ represents hydroxy, methoxy, ethoxy or allyloxy;

Rₙ represents hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl;

Rₒ represents hydroxy, morpholino, dimethylamino, piperidinyl or pyrrolidinyl;

Rₚ represents (S)-hydroxy or hydroxy;

R_{q} represents hydrogen or chloro;

Rᵣ represents hydrogen, methyl, ethyl, isopropyl or n-propyl;

Rₛ represents hydrogen, methyl, ethyl, or isopropyl.

The preparation method of the Formula I comprises

1) Preparing a compound of Formula 3 from a compound of Formula 2 which reacts with substituted boronic acid or tributylstannane by Suzuki-coupling or Stille cross-coupling in proper temperature or solvent (Step 1);

2) Preparing a compound of Formula 4 from the compound of Formula 3 by reduction, substitution, cyclization, reductive amination, hydrolysis, oxidation, dehydration, alcoholysis, or deacetylation (Step 2);

3) Preparing the desired compound represented by Formula I from the compound of Formula 4 which reacts with BCl₃ in the meaning of benzyl group deprotection (Step 3).

Each step of the above preparation method is described in more detail as follows.

1) Preparing a compound of Formula 2 as a start substance The compound of Formula 2 used as a start substance in Step 1 can be prepared using a known method (Paul A. Brough et al. J. Med. Chem. (2008), 51, 196-218).

2) Step 1: Preparing a compound of Formula 3 Step 1 of the preparation method is preparing a compound of Formula 3 from the compound of Formula 2.

If the group R₄ of Formula 3 is

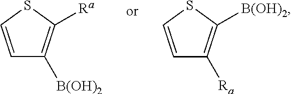

the compound of Formula 3 can be prepared by Suzuki cross-coupling in the above Step 1. A palladium(II)-catalyzed Suzuki cross-coupling reaction is carried out with unsubstituted or substituted boronic acid.
Dichlorobis(triphenylphosphine)palladium(II) is preferred. Examples of solvents useful in the reaction include N,N-dimethylformamide and H₂O. The reaction is heated to reflux for 2˜3 h under N₂, so as to obtain the compound of Formula 3.

Example of preparing the compound of Formula 3 from the compound of Formula 2 by Suzuki cross-coupling in the above Step 1 is illustrated below.

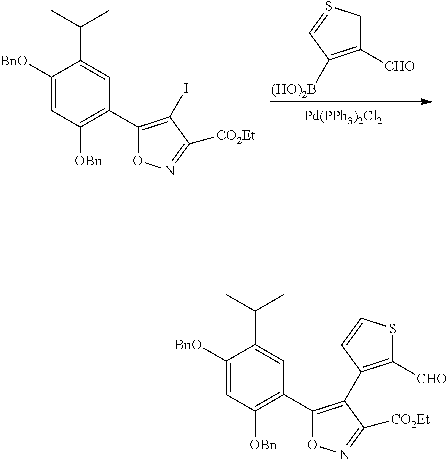

Besides, if the group R₄ of Formula 3 is cyano,

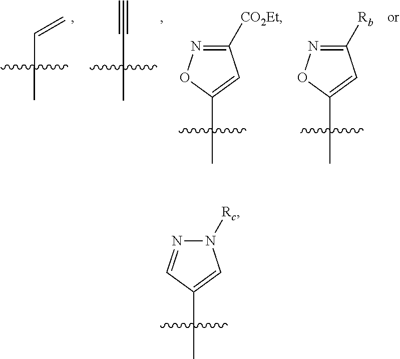

the compound of Formula 3 can be prepared by Stille cross-coupling in the above Step 1.
In Step 1, a palladium(0)-catalyzed Stille cross-coupling reaction is carried out in anhydrous CH₃CN or toluene with vinyl butylstannane

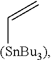

ethynyl tributylstannane

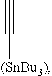

ethyl 5-(tributylstannyl)isoxazole-3-carboxylate

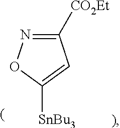

isoxazolyl tributylstannane substituted with R_{b}

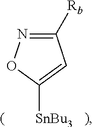

or pyrazolyl tributylstannane substituted with R_{c}

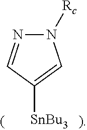

Preferred palldium(0) species is tetrakis(triphenylphosphine)palladium(0). The reaction is heated to reflux from 2.5 h to overnight under N₂, so as to obtain the compound of Formula 3. (R_{b} represents methyl, thiophenyl or phenyl, R_{c} represents hydrogen, trityl, methyl, ethyl or isopropyl)

An example of preparing the compound of Formula 3 from the compound of Formula 2 by Stille cross-coupling in the above Step 1 is illustrated below.

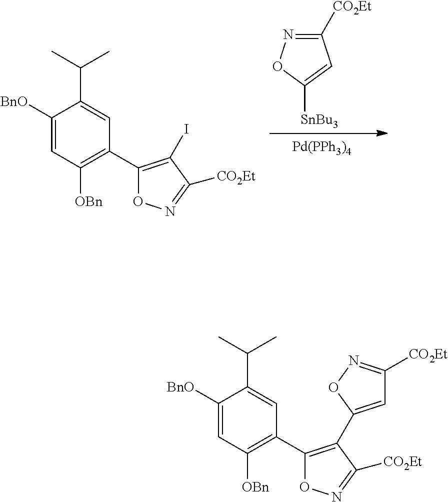

3) Step 2: Preparing a compound of Formula 4

In Step 2 of the preparation method, the compound of Formula 4 can be prepared from the compound of Formula 3 by reduction, substitution, cyclization, reductive amination, hydrolysis, oxidation, dehydration, alcoholysis, or deacetylation.

In the present invention, the compound of Formula 4 is prepared by reduction in which lithium aluminum chloride, lithium borohydride, or triphenylphosphine in tetrahydrofuran is used. The reaction is carried out from 2 h to overnight in 0° C. or 65° C. under N₂, so as to obtain the compound of Formula 4.

Besides, the compound of Formula 4 can be prepared from the compound of Formula 3 by substitution in the above Step 2.

First, in the above substitution reaction, mesylate compound is prepared from alcohol compound by reaction with methanesulfonyl chloride in methylene chloride, N,N-dimethylformamide, methanol, ethanol, or acetonitile. And then, desired functional group can be introduced by reaction of mesylate compound with unsubstituted or substituted alkylamine, unsubstituted or substituted cyclic amine, allylamine, potassium cyanide, potassium fluoride, phthalimide potassium salt, morpholine or thiomorpholine. An example of preparing the compound of Formula 4 from the compound of Formula 3 by substitution in the present invention is illustrated below.

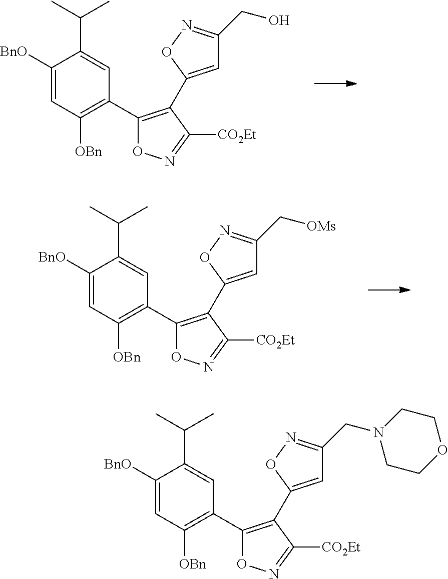

Second, in Step 2 of the present invention, azido compound is prepared from mesylate compound by reaction with sodium azide. And then, another compound of Formula 4 can be prepared from amine, which is derived from reduction of azido compound, by substitution using acetyl chloride or propionyl chloride in order to introduce substitution group of amine. An example is illustrated below.

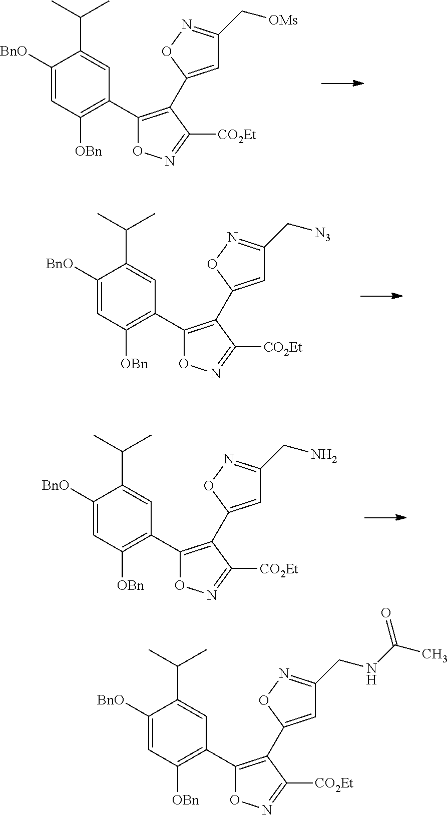

Third, in Step 2 of the present invention, amino hydroxylamine intermediate, another compound of Formula 4, can be prepared from cyano compound, the compound of Formula 3, by substitution using hydroxylamine. An example of preparing the compound of Formula 4 from the compound of Formula 3 by substitution in the present invention is illustrated below.

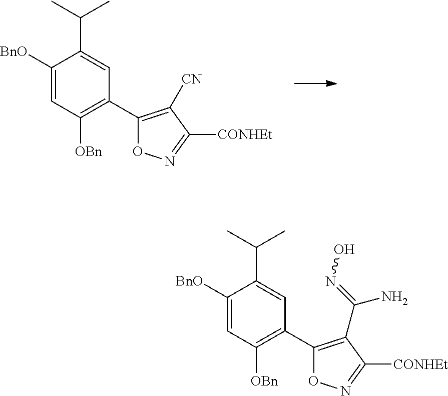

Fourth, in Step 2 of the present invention, acetylene compound, the compound of Formula 3, is converted to acetyl compound by substitution using formic acid. And then, dimethylamino-acryloyl compound, another compound of Formula 4, can be prepared from acetyl compound using N,N-dimethylformamide dimethyl acetal. An example of the above reaction is illustrated below.

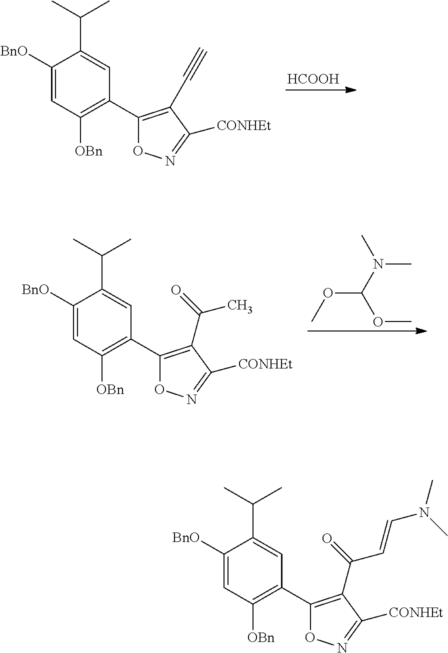

Fifth, alcohol compound is converted to acetate compound using methyl bromoacetate. And also, amine compound, another compound of Formula 4, can be prepared from trichloromethyl compound, the compound of Formula 3, by substitution using ammonia water, unsubstituted, or substituted amine. This method uses substitution. The above reaction is carried out from 1 h to overnight in 0° C. or reflux under N₂, so as to obtain the compound of Formula 4.

Besides, the compound of Formula 4 can be prepared from the compound of Formula 3 by cyclization in the above Step 2.

First, in the above cyclization reaction, isoxazoline, the compound of Formula 4, can be prepared from vinyl compound by reaction with ethyl 2-chloro-2-(hydroxyimino)acetate in toluene, methanol, ethanol, methylene chloride, pyridine, acetone, N,N-dimethylformamide, or acetonitile. An example of the above reaction is illustrated below.

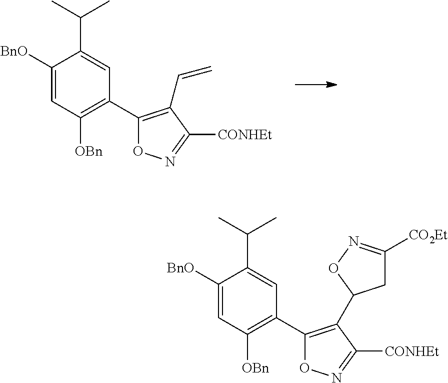

Second, unsubstituted or substituted oxadiazole compound, the compound of Formula 4, can be prepared from amino hydroxyimine compound by reaction with acetic anhydride, trifluoroacetic anhydride, trichloroacetic anhydride, ethyl chlorooxoacetate, propionyl chloride, 2-furoyl chloride, isobutyryl chloride, methoxyacetyl chloride, acetoxyacetyl chloride, 2-thiophenecarbonyl chloride, ethyl chloroformate, (S)-(−)-2-acetoxypropionyl chloride, unsubstituted or substituted benzoyl chloride, cycloalkanecarbonyl chloride, acryloyl chloride, trimethyl orthoformate and p-toluenesulfonic acid monohydrate, or 1,1-thiocarbonylimidazole and 1,8-diazabicyclo[5,4,0]unde-7-cene. An example of the above reaction is illustrated below.

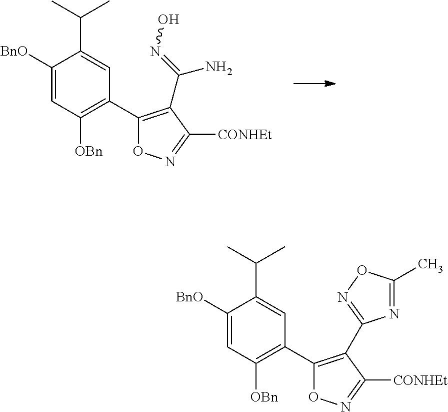

Third, tetrazole compound can be prepared from cyano compound using sodium azide and zinc(II) chloride. And pyrazole compound can be formed from dimethylamino-acryloyl compound using hydrazine monohydrate. Also, epoxide compound can be constructed from vinyl compound using hydrogen peroxide. Moreover, triazole compound can be synthesized from azido compound using vinyl acetate. The above reactions are carried out from 1.5 h to 67 h in 0° C., RT, or reflux, so as to obtain the compound of Formula 4.

Besides, the compound of Formula 4 can be prepared from the compound of Formula 3 by reductive amination in Step 2. In the above reductive amination, aldehyde compound is able to react with morpholine and sodium cyanoborohydride(NaCNBH₃) in methylene chloride. The reaction is carried out overnight in RT under N₂, so as to obtain the compound of Formula 4.

Besides, the compound of Formula 4 can be prepared from the compound of Formula 3 by hydrolysis in Step 2. In the above hydrolysis reaction, acid compound can be prepared from ethyl carboxylate compound by reaction with lithium hydroxide(LiOH) in mixture of tetrahydrofuran and H₂O. The reaction is carried out for 1 h in 0° C., so as to obtain the compound of Formula 4.

Besides, the compound of Formula 4 can be prepared from the compound of Formula 3 by oxidation in Step 2. In the above oxidation reaction, aldehyde compound can be prepared from alcohol compound by reaction with pyridinium chlorochromate(PCC) in methylene chloride. The reaction is carried out overnight in RT, so as to obtain the compound of Formula 4.

Besides, the compound of Formula 4 can be prepared from the compound of Formula 3 by dehydration in Step 2. In the above dehydration reaction, cyano compound can be prepared from amide compound by reaction with thionyl chloride in mixture of N,N-dimethylformamide/methylene chloride and methanol. Also, alkoxy imine compound can be formed from aldehyde compound using unsubstituted or substituted alkyloxyamine, and O-allyhydroxylimine compound can be obtained from aldehyde compound using O-allylhydroxylamine hydrochloride. The above reactions are carried out from 1 h to overnight in RT, so as to obtain the compound of Formula 4.

Besides, the compound of Formula 4 can be prepared from the compound of Formula 3 by alcoholysis in Step 2. In the above alcoholysis reaction, alcohol compound can be prepared from acetoxy compound using potassium carbonate in methanol. The reaction is carried out for 30 min in RT, so as to obtain the compound of Formula 4.

Besides, the compound of Formula 4 can be prepared from the compound of Formula 3 by deacetylation in the above Step 2. In the above deacetylation reaction, deacetylated alcohol compound is prepared from acetoxy compound using potassium carbonate in methanol. The reaction is carried out for 30 min in RT, so as to obtain the compound of Formula 4.

4) Step 3: Preparing a compound of Formula I,

In Step 3 of the preparation method, the derivatives of Formula I, desired compounds, are prepared from the compounds of Formula 4 by deprotection of benzyl group. The deprotection is carried out with Pd/C, ammonium formate, or boron trichloride (BCl₃) in Step 3 of the present invention, so as to obtain the derivatives of Formula I which are desired compounds. Using BCl₃ in dichloromethane is preferred. It is desirable that reaction should be carried out for 10 min in 0° C. and from 10 min to 1 h in RT.

In another aspect, the present invention provides a prodrug, represented by Formula II, of the compound of Formula I.

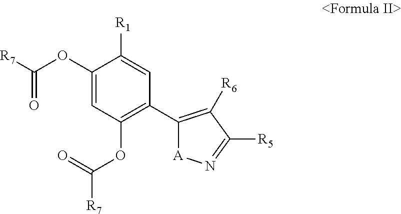

wherein, R₇ is acetyl, butyryl, 5-oxopentanoic acid, (tert-butoxycarbonyl)prolinyl, (tert-butoxycarbonyl)alaninyl, 5-(2,3-bis(tert-butoxycarbonyl)guanidino)-2-(tert-butoxycarbonyl)pentanoyl or (tert-butoxycarbonyl)valinyl, and A, R₁, R₅ or R₆ are each as defined above.
Particularly desirable examples of the prodrug of the above Formula I according to the present invention are shown below.
4-(3-(Ethylcarbamoyl)-4-(5-methyl-1,2,4-oxadiazol-3-yl)isoxazol-5-yl)-6-isopropyl-1,3-phenylene diacetate; (II-1)
4-(3-(Ethylcarbamoyl)-4-(5-methyl-1,2,4-oxadiazol-3-yl)isoxazol-5-yl)-6-isopropyl-1,3-phenylene dibutyrate; (II-2)
5-(4-(3-(Ethylcarbamoyl)-4-(5-methyl-1,2,4-oxadiazol-3-yl)isoxazol-5-yl)-5-hydroxy-2-isopropylphenoxy)-5-oxopentanoic acid; (II-3)
(2S,2′S)-1-tert-Butyl^{′2},2-4-(3-(ethylcarbamoyl)-4-(5-methyl-1,2,4-oxadiazol-3-yl)isoxazol-5-yl)-6-isopropyl-1,3-phenylene dipyrrolidine-1,2-dicarboxylate; (II-4)
(2S,2′S)-4-(3-(Ethylcarbamoyl)-4-(5-methyl-1,2,4-oxadiazol-3-yl)isoxazol-5-yl)-6-isopropyl-1,3-phenylene bis(2-(tert-butoxycarbonylamino)propanoate); (II-5)
(2S,2′S)-4-(3-(Ethylcarbamoyl)-4-(5-methyl-1,2,4-oxadiazol-3-yl)isoxazol-5-yl)-6-isopropyl-1,3-phenylene
bis(5-(2,3-bis(tert-butoxycarbonyl)guanidino)-2-(tert-butoxycarbonylamino)pentanoate); (II-6)
(2S,2′S)-4-(3-(Ethylcarbamoyl)-4-(5-methyl-1,2,4-oxadiazol-3-yl)isoxazol-5-yl)-6-isopropyl-1,3-phenylene
bis(2-(tert-butoxycarbonylamino)-3-methylbutanoate); (II-7)
(2S,2′S)-1-tert-Butyl^{′2},2-4-(3-(ethylcarbamoyl)-4-(1-methyl-1H-pyrazol-3-yl)isoxazol-5-yl)-6-isopropyl-1,3-phenylene dipyrrolidine-1,2-dicarboxylate; (II-8)
5-(4-(3-(Ethylcarbamoyl)-4-(1-methyl-1H-pyrazol-3-yl)isoxazol-5-yl)-5-hydroxy-2-isopropylphenoxy)-5-oxopentanoic acid; (II-9)

The compound of Formula II, the prodrug of the compound of Formula I, can be prapared by a reaction with amino acids, acyl chlorides, or acid anhydride. The reaction is carried out with Boc-protected amino acids, such as Boc-Pro-OH, Boc-Ala-OH, Boc-Arg(Boc)₂-OH or Boc-Val-OH; acyl chlorides such as acetyl chloride or butyryl chloride; acid anhydride such as glutaric anhydride and 4-(dimethylamino)pyridine/1-(3-dimethylaminopropyl)-3-ethylcarbodiimide hydrochloride in tetrahydrofuran or dichloromethane. Also, the reaction is carried out overnight in RT, so as to obtain the compound of Formula II.

An example of preparing the compound of Formula the prodrug of the compound of Formula I, is illustrated below.

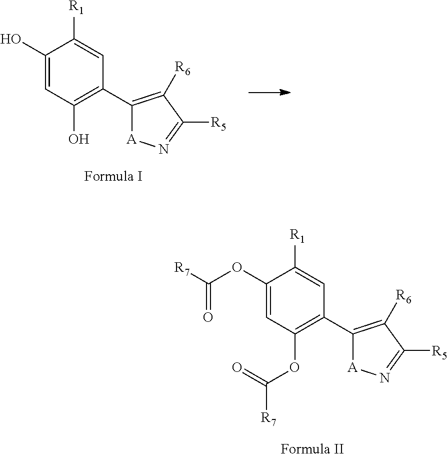

In another aspect, the present invention includes the compound represented by the above Formula I, a tautomer, or a pharmaceutically acceptable salt thereof, and provides a pharmaceutical composition for an antitumor agent including a pharmaceutically acceptable carrier.

The pharmaceutical composition of this invention for an antitumor agent, the orally administrable composition, may be in the form of tablets, capsules, powders, granules, lozenges, liquid or gel preparations, such as oral, topical, or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents. For topical application to the skin, the drug may be made up into a cream, lotion or ointment. Cream or ointment formulation which may be used for the drug are conventional formulations well known in the art, for examples as described in standard textbooks of pharmaceutics such as the British Pharmacopoeia. The active ingredient may also be administered parenterally in a sterile medium. Depending on the vehicle and concentration used, the drug can either be suspended or dissolved in the vehicle. Advantageously, adjuvants, such as a local anaesthetic, preservative and buffering agents, can be dissolved in the vehicle.

Compounds of the invention are also useful for in vitro assays dependent on inhibition of HSP90 activity, for example in screening for alternative classes of HSP90 inhibitors wherein the test compound competes with or displaces a compound of this invention. Accordingly, in yet another aspect, the invention includes a method of inhibiting HSP90 activity, comprising bringing into contact, in vitro, an HSP90 enzyme and a compound of Formula I as defined and specified above.

The goal of this invention provides to HSP90 inhibitors of Formula I having 5-membered heterocycle. The analogues which involved heterocycle induced resorcinol derivatives show effective anti-tumor activity in the many cancer cell line.

Accordingly, the invention also provides a method of treatment of diseases or conditions responsive to inhibition of HSP90 activity in mammals which method comprises administering to the mammal an amount of a compound of Formula I, effective to inhibit said HSP90 activity.

The used in vivo and method of the invention could be useful in the treatment of diseases which are responsive to inhibition of HSP90 activity such as immunosupression, Rheumatoid arthritis, Asthma, MS, Type I Diabetes, Lupus, Psoriasis, inflammatory Bowel Diseases, viral Diseases; diabetic retinopathy, hemangiomas, endometriosis; normal cells protection against chemotherapy-induced toxicity; protection from hypoxia-ischemic injury due to elevation of HSP70 in the heart and brain, scrapie/CJD, Huntingdon's and Alzhiemer's. Especially, it could be useful in the treatment of cancer.

The dosage of pharmaceutical composition of the present invention may vary depending on the patient's weight, age, gender, physical condition, diet, the time and mode of administration, excretion rates, and the severity of illness. The dosage of detailed drug composition may be administered in an effective amount ranging from 0.1 to 1000 mg on adult.

### Advantageous Effects

The present invention provides the novel 5-membered heterocycle derivatives, a tautomer, or a pharmaceutically approved salt thereof containing a compound having a superior HSP90 inhibitory activity.

The present invention can provide a novel drug composition containing a compound having a superior HSP90 inhibitory activity or a pharmaceutically approved salt thereof as an active ingredient, in particular a therapeutic agent for cancer.

A novel 5-membered heterocycle derivative of the present invention, a tautomer, or a pharmaceutically acceptable salt thereof can be used very effectively to treat various diseases, treated or prevented by inhibition of HSP90 activity, especially several carcinomas including ovarian and gastric cancer.

### BEST MODE

The present invention will be described more particularly by the Examples but the present invention is not limited at all by these examples.

As can be seen from Table 1˜11, Most of the compounds of the present invention exhibited significant antitumor activity for ATPase inhibitory actvity and MTT assay.

### Example 1

### 5-(2,4-Dihydroxy-5-isopropylphenyl)-N-ethyl-4-(5-(morpholinomethyl)thiophen-2-yl)isoxazole-3-carboxamide (I-1)

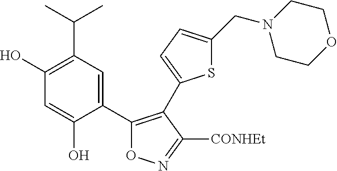

### Step 1

### 5-(2,4-Bis(benzyloxy)-5-isopropylphenyl)-N-ethyl-4-(5-formylthiophen-2-yl)isoxazole-3-carboxamide

NaHCO₃ (376 mg, 4.47 mmol) was added to a solution of 5-(2,4-bis(benzyloxy)-5-isopropylphenyl)-N-ethyl-4-iodoisoxazole-3-carboxamide (890 mg, 1.49 mmol) and 5-formylthiophene-2-ylboronic acid (465.5 mg, 2.98 mmol) in DMF (6.19 ml)/H₂O (1.21 ml) under N₂. After 10 min, dichorobis(triphenylphosphine)palladium(II) (523 mg, 0.745 mmol) was added, and the suspension was heated to reflux for 2˜3 h. The reaction mixture was cooled to ambient temperature, solvent was evaporated in vacuo. The residue was extracted between methylene chloride and water, and the organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(5-formylthiophen-2-yl)isoxazole-3-carboxamide (360.6 mg, 0.62 mmol) in a yield of 42%.

¹H-NMR (400 MHz, CDCl₃) δ 9.91 (s, 1H), 7.57-7.24 (m, 9H), 7.09-7.04 (m, 2H), 6.82 (t, 1H), 6.50 (s, 1H), 5.03 (d, 2H), 4.83 (s, 2H), 3.47 (m, 2H), 3.27 (m, 1H), 1.25 (t, 3H), 1.23-1.15 (m, 6H)

### Step 2

### 5-(2,4-Bis(benzyloxy)-5-isopropylphenyl)-N-ethyl-4-(5-(morpholinomethyl)thiophen-2-yl)isoxazole-3-carboxamide

After 1 h, acetic acid (850.2 μl, 14.8 mmol) was added dropwise to a mixture of the intermediate compound (Step 1) (1.73 g, 2.97 mmol), morpholine (761.8 μl, 8.9 mmol), sodium cyanoborohydride (NaCNBH₃) (373.7 mg, 5.9 mmol), molecular sieves 4 Å (1.68 g), and methylene chloride (50.6 ml). The reaction mixture was left to stir at RT under a nitrogen atmosphere for overnight, and the residue was extracted between methylene chloride and water. The organic phase was dried with magnesium sulfate, and evaporated in vacuo. The residue was purified by silica gel column chromatography to afford the intermediate compound 5-(2,4-Bis(benzyloxy)-5-isopropylphenyl)-N-ethyl-4-5-(morpholinomethyl) thiophen-2-yl)isoxazole-3-carboxamide (1.13 g, 1.73 mmol) in a yield of 58%.

¹H-NMR (400 MHz, CDCl₃) δ 7.39-7.16 (m, 12H), 7.07 (d, 1H), 6.71 (s, 1H), 6.52 (s, 1H), 5.02 (s, 2H), 4.94 (s, 2H), 3.66-3.61 (m, 6H), 3.47 (q, 2H), 3.26 (m, 1H), 2.43 (d, 4H), 1.23 (t, 3H), 1.11 (d, 6H)

### Step 3

### 5-(2,4-Dihydroxy-5-isopropylphenyl)-N-ethyl-4-(5-(morpholinomethyl)thiophen-2-yl)isoxazole-3-carboxamide

To the intermediate compound (Step 2) (1.13 g, 1.73 mmol) in methylene chloride (34.5 ml) cooled to 0° C. under N₂ was added boron tirchloride (BCl₂) (1.0M in methylene chloride, 8.67 ml, 8.67 mmol). The reaction was allowed to warm to RT and was stirred for 10 min. After this time, methanol was added, the mixture was concentrated. The residue was purified by silica gel column chromatography to afford the title compound (311.5 mg, 0.66 mmol) in a yield of 38%.

¹H-NMR (400 MHz, CDCl₃) δ 7.04 (s, 1H), 7.00 (d, 1H), 6.89 (t, 1H), 6.83 (d, 1H), 6.19 (s, 1H), 3.66-3.63 (m, 6H), 3.43 (m, 2H), 3.23-3.06 (m, 1H), 2.51 (s, 4H), 1.28-1.19 (t, 3H), 1.03 (d, 6H)

### Example 2

### 5-(2,4-Dihydroxy-5-isopropylphenyl)-N-ethyl-4-(2-(morpholinomethyl)thiophen-3-yl)isoxazole-3-carboxamide (I-2)

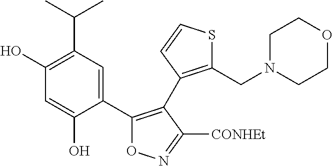

### Step 1

### 5-(2,4-Bis(benzyloxy)-5-isopropylphenyl)-N-ethyl-4-(2-formylthiophen-3-yl)isoxazole-3-carboxamide

This compound was made using the procedure described for example 1 (Step 1). Thus, 5-(2,4-bis(benzyloxy)-5-isopropylphenyl)-N-ethyl-4-iodoisoxazole-3-carboxamide (122.5 mg, 0.2 mmol) was reacted with 2-formylthiophen-3-ylboronic acid (64 mg, 0.41 mmol) and NaHCO₃ (51.8 mg, 0.616 mmol) to afford the intermediate compound 5-(2,4-bis(benzyloxy)-5-isopropylphenyl)-N-ethyl-4-(2-formylthiophen-3-yl)iso xazole-3-carboxamide (78.8 mg, 0.135 mmol) in a yield of 68%.

¹H-NMR (400 MHz, CDCl₃) δ 9.46 (s, 1H), 7.57-7.24 (m, 9H), 7.09-7.04 (m, 2H), 6.85 (t, 1H), 6.43 (s, 1H), 4.96 (d, 2H), 4.80 (s, 2H), 3.42 (m, 2H), 3.27 (m, 1H), 1.22 (t, 3H), 1.09-1.07 (m, 6H)

### Step 2

### 5-(2,4-Bis(benzyloxy)-5-isopropylphenyl)-N-ethyl-4-(2-(morpholinomethyl)thiophen-3-yl)isoxazole-3-carboxamide

this intermediate compound (Step 1) was made using the procedure described for example 1 (Step 2), using morpholine (21.2 μl, 0.248 mmol) and NaCNBH₃ (10.39 mg, 0.16 mmol) in reaction with this compound (48 mg, 0.083 mmol). The crude product was purified by silica gel column chromatography to afford the intermediate compound 5-(2,4-bis(benzyloxy)-5-isopropylphenyl)-N-ethyl-4-(2-(morpholinomethyl)thiophen-3-yl)isoxazole-3-carboxamide (35.5 mg, 0.054 mmol) in a yield of 66%.

¹H-NMR (400 MHz, CDCl₃) δ 7.39-7.26 (m, 10H), 7.21 (d, 1H), 6.99-6.95 (m, 2H), 6.77 (s, 1H), 6.47 (s, 1H), 4.96 (s, 4H), 3.52 (t, 4H), 3.41 (t, 2H), 3.23-3.17 (m, 3H), 2.16 (s, 4H), 1.20 (t, 3H), 1.02 (d, 6H)

### Step 3

### 5-(2,4-Dihydroxy-5-isopropylphenyl)-N-ethyl-4-(2-(morpholinomethyl)thiophen-3-yl)isoxazole-3-carboxamide

This compound was made using the procedure described for example 1 (Step 3). Thus, this intermediate compound (Step 2) (48.8 mg, 0.0749 mmol) was reacted with BCl₃ to afford a crude product, which was purified by silica gel column chromatography to afford the title compound (35.1 mg, 0.74 mmol) in a yield of 99%.

¹H-NMR (400 MHz, CDCl₃) δ 7.31 (d, 1H), 7.00 (d, 1H), 6.80 (s, 1H), 6.30 (s, 1H), 3.56 (t, 4H), 3.42-3.34 (m, 4H), 3.05 (m, 1H), 2.24 (s, 4H), 1.26 (t, 3H), 1.03 (d, 6H)

### Example 3

### 4-(3-(Hydroxymethyl)-4-(thiophen-3-yl)isoxazol-5-yl)-6-isopropylbenzene-1,3-diol (I-3)

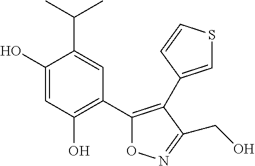

### Step 1

### Ethyl 5-(2,4-bis(benzyloxy)-5-isopropylphenyl)-4-iodoisoxazole-3-carboxylate

Ethyl 5-(2,4-bis(benzyloxy)-5-isopropylphenyl)isoxazole-3-carboxylate (1.5 g, 3.18 mmol) was suspended in CH₃CN (40 ml), and treated with N-iodosuccinimide (2.15 g, 9.54 mmol) followed by ceric ammonium nitrate(IV) (174 mg, 0.32 mmol). The reaction mixture was heated to reflux for 18 h, and quenched with saturated sodium thiosulfate solution, solvent was evaporated in vacuo. The residue was extracted between methylene chloride and water. The organic phase was dried with magnesium sulfate, and evaporated in vacuo. The residue was purified by silica gel column chromatography to afford the intermediate compound ethyl 5-(2,4-bis(benzyloxy)-5-isopropylphenyl)-4-iodoisoxazole-3-carboxylate (1.47 g, 2.46 mmol) in a yield of 77%.

¹H-NMR (400 MHz, CDCl₃) δ 7.43-7.28 (m, 11H), 6.59 (s, 1H), 5.08 (s, 2H), 5.06 (s, 2H), 4.49 (q, 2H), 3.34 (m, 1H), 1.45 (t, 3H), 1.23 (d, 6H)

### Step 2

### Ethyl 5-(2,4-bis(benzyloxy)-5-isopropylphenyl)-4-(thiophen-3-yl)isoxazole-3-carboxylate

This compound was made using the procedure described for example 1 (Step 1). Thus, the intermediate compound (Step 1) (1.4 g, 2.34 mmol) was reacted with dichorobis(triphenylphosphine)palladium(II) (822 mg, 1.17 mmol), thiophen-3-ylboronic acid (524 mg, 4.69 mmol) and NaHCO₃ (591 mg, 7.03 mmol) to afford the intermediate compound Ethyl 5-(2,4-bis(benzyloxy)-5-isopropylphenyl)-4-(thiophen-3-yl)isoxazole-3-carboxy late (940 mg, 1.70 mmol) in a yield of 72%.

¹H-NMR (400 MHz, CDCl₃) δ 7.41-7.25 (m, 10H), 7.22 (dd, 1H), 7.16 (m, 1H), 7.13 (s, 1H), 6.98 (dd, 1H), 6.49 (s, 1H), 5.00 (s, 2H), 4.85 (s, 2H), 4.40 (q, 2H), 3.25 (sept, 1H), 1.37 (t, 3H), 1.10 (d, 6H)

### Step 3

### (5-(2,4-Bis(benzyloxy)-5-isopropylphenyl)-4-(thiophen-3-yl)isoxazol-3-yl)methanol

To the intermediate compound (Step 2) (940 mg, 1.70 mmol) in THF (15 ml) cooled to 0° C. under N₂ was added lithium aluminium hydride (97 mg, 2.55 mmol). The reaction was allowed to warm to RT and was stirred for 4 h. After this time, the reaction mixture was cooled to 0° C. and sequentially water (0.1 ml), 10% NaOH aqueous solution (0.2 ml), and water (0.3 ml) were added. The reaction was allowed to warm to RT, and diethylether (15 ml) was added. After being stirred for 30 min, the reaction mixture was filtered through a pad of Celite, and the filtrate was concentrated. The residue was purified by silica gel column chromatography to afford the intermediate compound (5-(2,4-bis(benzyloxy)-5-isopropylphenyl)-4-(thiophen-3-yl)isoxazol-3-yl)meth anol (600 mg, 1.17 mmol) in a yield of 69%.

¹H-NMR (400 MHz, CDCl₃) δ 7.40-7.23 (m, 11H), 7.10 (m, 2H), 6.95 (dd, 1H), 6.52 (s, 1H), 5.02 (s, 2H), 4.84 (s, 2H), 4.82 (d, 2H), 3.29 (sept, 1H), 2.09 (t, 1H), 1.14 (d, 6H)

### Step 4

### 4-(3-(Hydroxymethyl)-4-(thiophen-3-yl)isoxazol-5-yl)-6-isopropylbenzene-1,3-diol

This compound was made using the procedure described for example 1 (Step 3). Thus, this intermediate compound (Step 3) (70 mg, 0.14 mmol) was reacted with BCl₃ to afford a crude product, which was purified by silica gel column chromatography to afford the title compound (40 mg, 0.12 mmol) in a yield of 88%.

¹H-NMR (400 MHz, DMSO-d₆) δ 9.71 (s, 1H), 9.57 (s, 1H), 7.59 (dd, 1H), 7.50 (dd, 1H), 7.00 (dd, 1H), 6.86 (s, 1H), 6.46 (s, 1H), 5.58 (t, 1H), 4.55 (d, 2H), 3.05 (sept, 1H), 1.03 (d, 6H)

### Example 4

### N-((5-(2,4-Dihydroxy-5-isopropylphenyl)-4-(thiophen-3-yl)isoxazol-3-yl)methyl) propionamide (I-4)

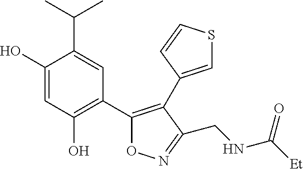

### Step 1

### (5-(2,4-Bis(benzyloxy)-5-isopropylphenyl)-4-(thiophen-3-yl)isoxazol-3-yl)methyl methanesulfonate

To (5-(2,4-bis(benzyloxy)-5-isopropylphenyl)-4-(thiophen-3-yl)isoxazol-3-yl)meth anol (100 mg, 0.19 mmol) in methylene chloride (30) cooled to 0° C. under N₂ was added triethylamine (81.7 μl, 0.57 mmol) and methanesulfonyl chloride (30.2 μl, 0.39 mmol). The reaction was allowed to warm to RT and was stirred for 1.5 h and the residue was extracted between methylene chloride and water. The organic phase was dried with magnesium sulfate, and evaporated in vacuo. The residue was purified by silica gel column chromatography to afford the intermediate compound

(5-(2,4-bis(benzyloxy)-5-isopropylphenyl)-4-(thiophen-3-yl)-isoxazol-3-yl)methyl methanesulfonate (95 mg, 0.16 mmol) in a yield of 82%.

¹H-NMR (400 MHz, CDCl₃) δ 7.40-7.23 (m, 11H), 7.10 (dd, 2H), 6.92 (dd, 1H), 6.52 (s, 1H), 5.35 (s, 2H), 5.03 (s, 2H), 4.82 (s, 2H), 3.28 (sept, 1H), 2.96 (s, 3H), 1.14 (d, 6H)

### Step 2

### 3-(Azidomethyl)-5-(2,4-bis(benzyloxy)-5-isopropylphenyl)-4-(thiophen-3-yl)isoxazole

To the intermediate compound (Step 1) (173 mg, 0.29 mmol) in DMF (30) was added sodium azide (76 mg, 1.17 mmol), and the reaction mixture was heated for 6 h at 65° C. The mixture was cooled to ambient temperature, solvent was evaporated in vacuo. The residue was extracted between ethyl acetate and water. The organic phase was dried with magnesium sulfate, and evaporated in vacuo. The residue was purified by silica gel column chromatography to afford the intermediate compound 3-(azidomethyl)-5-(2,4-bis(benzyloxy)-5-isopropylphenyl)-4-(thiophen-3-yl)iso xazole (135 mg, 0.25 mmol) in a yield of 86%.

¹H-NMR (400 MHz, CDCl₃) δ 7.40-7.23 (m, 11H), 7.13 (m, 2H), 6.91 (dd, 1H,), 6.51 (s, 1H), 5.02 (s, 2H), 4.84 (s, 2H), 4.43 (s, 2H), 3.28 (sept, 1H), 1.14 (d, 6H)

### Step 3

### N-((5-(2,4-Bis(benzyloxy)-5-isopropylphenyl)-4-(thiophen-3-yl)isoxazol-3-yl)methyl)propionamide

Triphenylphosphine (50 mg, 0.19 mmol) was added to a solution of the intermediate compound (Step 2) (85 mg, 0.16 mmol) in THF (3 ml). After 1.5 h at RT, water (1.5 ml) was added, and the reaction mixture heat at 65° C. for 5 h. The mixture was cooled to ambient temperature, solvent was evaporated in vacuo. To the residue in methylene chloride (3 ml) cooled to 0° C. was added pyridine (27.9 μl, 0.34 mmol). Propionyl chloride (15.0 μl, 0.17 mmol) was added to the reaction mixture at the same condition, and then the mixture was left to stir at RT for 18 h. The residue was extracted between methylene chloride and water. The organic phase was washed with 2N—HCl, saturated aqueous NaCl, dried with magnesium sulfate, and evaporated in vacuo. The residue was purified by silica gel column chromatography to afford the intermediate compound N-((5-(2,4-bis(benzyloxy)-5-isopropylphenyl)-4-(thiophen-3-yl)isoxazol-3-yl)methyl)propionamide (50 mg, 0.09 mmol) in a yield of 66%.

¹H-NMR (400 MHz, CDCl₃) δ 7.38-7.21 (m, 10H), 7.14-7.09 (m, 3H), 6.87 (dd, 1H), 6.50 (s, 1H), 6.15 (br, 1H), 5.01 (s, 2H), 4.83 (s, 2H), 4.63 (d, 2H), 3.28 (sept, 1H), 2.25 (q, 2H), 1.16 (t, 3H), 1.13 (d, 6H)

### Step 4

### N-((5-(2,4-Dihydroxy-5-isopropylphenyl)-4-(thiophen-3-yl)isoxazol-3-yl)methyl)propionamide

This compound was made using the procedure described for example 1 (Step 3). Thus, this intermediate compound (Step 3) (50 mg, 0.09 mmol) was reacted with BCl₃ to afford a crude product, which was purified by silica gel column chromatography to afford the title compound (15 mg, 0.04 mmol) in a yield of 44%.

¹H-NMR (400 MHz, DMSO-d₆) δ 9.71 (s, 1H), 9.57 (s, 1H), 8.28 (br t, 1H), 7.52 (dd, 1H), 7.44 (dd, 1H), 6.93 (dd, 1H), 6.83 (s, 1H), 6.44 (s, 1H), 4.39 (d, 2H), 3.02 (sept, 1H), 2.08 (q, 2H), 1.00 (d, 6H), 0.96 (t, 3H)

### Example 5

### 4-(3-((1H-1,2,3-Triazol-1-yl)methyl)-4-(thiophen-3-yl)isoxazol-5-yl)-6-isopro pylbenzene-1,3-diol (I-5)

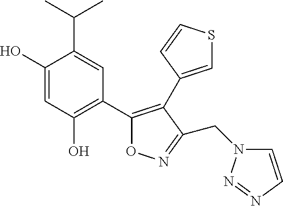

### Step 1

### 3-((1H-1,2,3-Triazol-1-yl)methyl)-5-(2,4-bis(benzyloxy)-5-isopropylphenyl)-4-(thiophen-3-yl)isoxazole

3-(Azidomethyl)-5-(2,4-bis(benzyloxy)-5-isopropylphenyl)-4-(thiophen-3-yl)iso xazole (65 mg, 0.12 mmol) was dissolved in vinyl acetate (3 ml), and heated at 100° C. for 6 h in sealtube. The reaction mixture was cooled to ambient temperature, solvent was evaporated in vacuo, and the residue was extracted between ethyl acetate and water. The organic phase was dried with magnesium sulfate, and evaporated in vacuo. The residue was purified by silica gel column chromatography to afford the intermediate compound 3-((1H-1,2,3-triazol-1-yl)methyl)-5-(2,4-bis(benzyloxy)-5-isopropylphenyl)-4-(thiophen-3-yl)isoxazole (60 mg, 0.11 mmol) in a yield of 88%.

¹H-NMR (400 MHz, CDCl₃) δ 7.71 (d, 1H), 7.66 (d, 1H), 7.39-7.34 (m, 5H), 7.28-7.21 (m, 5H), 7.10-7.07 (m, 3H), 6.76 (dd, 1H), 6.50 (s, 1H), 5.70 (s, 2H), 5.01 (s, 2H), 4.81 (s, 2H), 3.27 (sept, 1H), 1.12 (d, 6H)

### Step 2

### 4-(3-((1H-1,2,3-Triazol-1-yl)methyl)-4-(thiophen-3-yl)isoxazol-5-yl)-6-isopro pylbenzene-1,3-diol

This compound was made using the procedure described for example 1 (Step 3). Thus, this intermediate compound (Step 1) (55 mg, 0.10 mmol) was reacted with BCl₃ to afford a crude product, which was purified by silica gel column chromatography to afford the title compound (10 mg, 0.03 mmol) in a yield of 27%.

¹H-NMR (400 MHz, CD₃OD) δ 7.87 (d, 1H), 7.69 (d, 1H), 7.39 (dd, 1H), 7.29 (dd, 1H), 6.89 (s, 1H), 6.87 (dd, 1H), 6.35 (s, 1H), 5.80 (s, 2H), 3.08 (sept, 1H), 1.02 (d, 6H)

### Example 6

### N-((5-(2,4-Dihydroxy-5-isopropylphenyl)-4-(thiophen-3-yl)isoxazol-3-yl)methyl)acetamide (I-6)

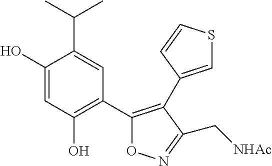

### Step 1

### N-((5-(2,4-Bis(benzyloxy)-5-isopropylphenyl)-4-(thiophen-3-yl)isoxazol-3-yl)methyl)acetamide

This compound was made using the procedure described for example 4 (Step 3). Thus, 3-(azidomethyl)-5-(2,4-bis(benzyloxy)-5-isopropylphenyl)-4-(thiophen-3-yl)isoxazole (60 mg, 0.11 mmol) was reacted with triphenylphosphine (35 mg, 0.13 mmol) to afford the intermediate compound N-((5-(2,4-bis(benzyloxy)-5-isopropylphenyl)-4-(thiophen-3-yl)isoxazol-3-yl)methyl)acetamide (40 mg, 0.072 mmol) in a yield of 65%.

¹H-NMR (400 MHz, CDCl₃) δ 7.41-7.24 (m, 9H), 7.21 (s, 1H), 7.13-7.09 (m, 3H), 6.87 (dd, 1H), 6.50 (s, 1H), 6.20 (br s, 1H), 5.01 (s, 2H), 4.83 (s, 2H), 4.61 (d, 2H), 3.27 (sept, 1H), 2.03 (s, 3H), 1.13 (d, 6H)

### Step 2

### N-((5-(2,4-Dihydroxy-5-isopropylphenyl)-4-(thiophen-3-yl)isoxazol-3-yl)methyl)acetamide

This compound was made using the procedure described for example 1 (Step 3). Thus, this intermediate compound (Step 1) was reacted with BCl₃ to afford a crude product, which was purified by silica gel column chromatography to afford the title compound (20 mg, 0.05 mmol) in a yield of 74%.

¹H-NMR (400 MHz, CD₃OD) δ 7.40 (dd, 1H), 7.33 (dd, 1H), 6.96 (dd, 1H), 6.88 (s, 1H), 6.36 (s, 1H), 4.51 (s, 2H), 3.11 (sept, 1H), 1.92 (s, 3H), 1.03 (d, 6H)

### Example 7

### Ethyl 5-(5-(2,4-dihydroxy-5-isopropylphenyl)-3-(ethylcarbamoyl)isoxazol-4-yl)-4,5-dihydroisoxazole-3-carboxylate (I-7)

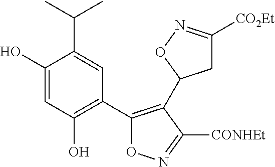

### Step 1

### 5-(2,4-Bis(benzyloxy)-5-isopropylphenyl)-N-ethyl-4-vinylisoxazole-3-carboxamide

Asoluntion 5-(2,4-bis(benzyloxy)-5-isopropylphenyl)-N-ethyl-4-iodoisoxazole-3-carboxamide (300 mg, 0.50 mmol) and tetrakis(triphenylphosphine)palladium(0) (29 mg, 0.03 mmol) in toluene (5 ml) heated at 110° C. After 10 min, tributyl(vinyl)tin (0.18 ml, 0.60 mmol) was added, and the suspension was heated to reflux for 2.5 h. The reaction mixture was cooled to ambient temperature, solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-Bis(benzyloxy)-5-isopropylphenyl)-N-ethyl-4-vinylisoxazole-3-carboxami de (240 mg, 0.48 mmol) in a yield of 96%.

¹H-NMR (400 MHz, CDCl₃) δ 7.41-7.24 (m, 11H), 6.85 (dd, 1H), 6.80 (t, 1H), 6.58 (s, 1H), 5.56 (dd, 1H), 5.25 (dd, 1H), 5.05 (s, 2H), 5.04 (s, 2H), 3.50 (m, 2H), 3.32 (sept, 1H), 1.26 (t, 3H), 1.20 (d, 6H)

### Step 2

### Ethyl

### 5-(5-(2,4-bis(benzyloxy)-5-isopropylphenyl)-3-(ethylcarbamoyl)isoxazol-4 yl)-4,5-dihydroisoxazole-3-carboxylate

To a solution of the intermediate compound (Step 1) (180 mg, 0.36 mmol) in methylene chloride (50) was added ethyl 2-chloro-2-(hydroxyimino)acetate (60 mg, 0.40 mmol) and potassium carbonate (55 mg, 0.40 mmol). The reaction mixture was stirred at RT for 16 h, and quenched with water. And then the residue was extracted between methylene chloride and water. The organic phase was dried with magnesiumsulfate, and evaporated in vacuo. The residue was purified by silica gel column chromatography to afford the intermediate compound ethyl

5-(5-(2,4-bis(benzyloxy)-5-isopropylphenyl)-3-(ethylcarbamoyl)isoxazol-4-yl)-4,5-dihydroisoxazole-3-carboxylate (190 mg, 0.31 mmol) in a yield of 86%.

¹H-NMR (400 MHz, CDCl₃) δ 7.41-7.22 (m, 11H), 6.75 (br t, 1H), 6.58 (s, 1H), 5.87 (t, 1H), 5.06-5.00 (m, 4H), 4.29 (q, 2H), 3.47-3.29 (m, 4H), 3.11 (dd, 1H), 1.34 (t, 3H), 1.28-1.20 (m, 9H)

### Step 3

### Ethyl

### 5-(5-(2,4-dihydroxy-5-isopropylphenyl)-3-(ethylcarbamoyl)isoxazol-4-yl)-4,5-dihydroisoxazole-3-carboxylate

This compound was made using the procedure described for example 1 (Step 3). Thus, this intermediate compound (Step 2) (35 mg, 0.06 mmol) was reacted with BCl₃ to afford a crude product, which was purified by silica gel column chromatography to afford the title compound (24 mg, 0.06 mmol) in a yield of 97%.

¹H-NMR (400 MHz, CD₃OD) δ 7.10 (s, 1H), 6.42 (s, 1H), 5.85 (dd, 1H), 4.30 (q, 2H), 3.50 (m, 2H), 3.36 (q, 2H), 3.19 (sept, 1H), 1.34 (t, 3H), 1.23-1.18 (m, 9H)

### Example 8

### 5-(2,4-Dihydroxy-5-isopropylphenyl)-N-ethyl-4-(3-(ethylcarbamoyl)-4,5-dihydroisoxazol-5-yl)isoxazole-3-carboxamide (I-8)

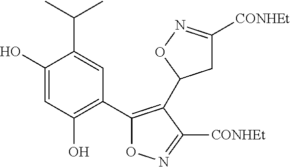

### Step 1

### 5-(2,4-Bis(benzyloxy)-5-isopropylphenyl)-N-ethyl-4-(3-(ethylcarbamoyl)-4,5-dihydroisoxazol-5-yl)isoxazole-3-carboxamide (I-8)

30˜40% Ethylamine in MeOH (1 ml) was added to a ethyl 5-(5-(2,4-bis(benzyloxy)-5-isopropylphenyl)-3-(ethylcarbamoyl)isoxazol-4-yl)-4,5-dihydroisoxazole-3-carboxylate (70 mg, 0.11 mmol), and the reaction mixture was heated to reflux for 1 h. The mixture was cooled to ambient temperature, solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(3-(ethylcarbamoyl)-4,5-dihydroisoxazol-5-yl)isoxazole-3-carboxamide (65 mg, 0.11 mmol) in a yield of 93%.

¹H-NMR (400 MHz, CDCl₃) δ 7.43-7.20 (m, 11H), 6.80 (br t, 1H), 6.56 (s, 1H), 6.54 (br t, 1H), 5.89 (t, 1H), 5.04 (s, 2H), 5.03 (s, 2H), 3.46 (m, 2H), 3.40-3.22 (m, 5H), 1.28-1.16 (m, 12H)

### Step 2

### 5-(2,4-Dihydroxy-5-isopropylphenyl)-N-ethyl-4-(3-(ethylcarbamoyl)-4,5-dihydroisoxazol-5-yl)isoxazole-3-carboxamide

This compound was made using the procedure described for example 1 (Step 3). Thus, this intermediate compound (Step 1) (60 mg, 0.10 mmol) was reacted with BCl₃ to afford a crude product, which was purified by silica gel column chromatography to afford the title compound (37 mg, 0.09 mmol) in a yield of 87%.

¹H-NMR (400 MHz, CD₃OD) δ 7.11 (s, 1H), 6.42 (s, 1H), 5.78 (t, 1H), 3.48 (dd, 1H), 3.36 (q, 2H), 3.19 (sept, 1H), 1.24-1.15 (m, 12H)

### Example 9

### 5-(2,4-Dihydroxy-5-isopropylphenyl)-N-ethyl-4-(3-(hydroxymethyl)-4,5-dihydroisoxazol-5-yl)isoxazole-3-carboxamide (I-9)

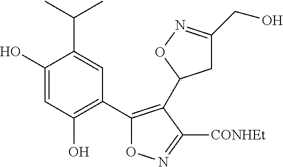

### Step 1

### 5-(2,4-Bis(benzyloxy)-5-isopropylphenyl)-N-ethyl-4-(4,5-dihydro-3-(hydroxymethyl)isoxazol-5-yl)isoxazole-3-carboxamide

To ethyl 5-(5-(2,4-bis(benzyloxy)-5-isopropylphenyl)-3-(ethylcarbamoyl)isoxazol-4-yl)-4,5-dihydroisoxazole-3-carboxylate (53 mg, 0.09 mmol) in THF (1 ml) cooled to 0° C. under N₂ was added lithium borohydride (3.8 mg, 0.17 mmol). The reaction was left to stir at RT for overnight. After this time, the reaction mixture was cooled to 0° C., and sequentially water and aqueous ammounium chloride solution were added. And the reaction mixture was extracted between ethyl acetate and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(4,5-dihydro-3-(hydroxymethyl)isoxazol-5-yl)isoxazole-3-carboxamide (30 mg, 0.05 mmol) in a yield of 61%.

¹H-NMR (400 MHz, CDCl₃) δ 7.41-7.26 (m, 11H), 6.76 (br t, 1H), 6.59 (s, 1H), 5.64 (dd, 1H), 5.07 (s, 2H), 5.01 (s, 2H), 4.40 (m, 1H), 4.21 (m, 1H), 3.44 (m, 2H), 3.35-3.29 (m, 2H), 2.86 (dd, 1H), 2.69 (br s, 1H), 1.26-1.21 (m, 9H)

### Step 2

### 5-(2,4-Dihydroxy-5-isopropylphenyl)-N-ethyl-4-(3-(hydroxymethyl)-4,5-dihydroisoxazol-5-yl)isoxazole-3-carboxamide

This compound was made using the procedure described for example 1 (Step 3). Thus, this intermediate compound (Step 1) (25 mg, 0.04 mmol) was reacted with BCl₃ to afford a crude product, which was purified by silica gel column chromatography to afford the title compound (15 mg, 0.04 mmol) in a yield of 88%.

¹H-NMR (400 MHz, CD₃OD) δ 7.10 (s, 1H), 6.42 (s, 1H), 5.62 (t, 1H), 4.28 (dd, 2H), 3.41-3.34 (m, 4H), 3.19 (sept, 1H), 1.23-1.18 (m, 9H)

### Example 10

### 5-(2,4-Dihydroxy-5-isopropylphenyl)-N-ethyl-4-(1H-tetrazol-5-yl)isoxazole-3-carboxamide (I-10)

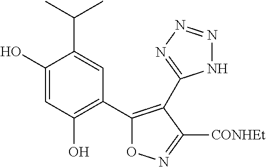

### Step 1

### 5-(2,4-Bis(benzyloxy)-5-isopropylphenyl)-N-ethyl-4-(1H-tetrazol-5-yl)isoxazole-3-carboxamide

To a solution of 5-(2,4-bis(benzyloxy)-5-isopropylphenyl)-4-cyano-N-ethylisoxazole-3-carboxamide (1.14 g, 2.30 mmol) in pyridine (230) cooled to 0° C. were added sodium azide (1.50 g, 23.0 mmol) and ZnCl₂ (1.57 g, 11.5 mmol) and the reaction mixuter was heated to reflux for overnight. After this time, the reaction mixture was cooled to RT, solvnet was concentrated. The reaction mixture was diluted with methylene chloride, and filtered through a pad of Celite 545. and the filtrate was washed with 2N—HCl, saturated aqueous NaCl, dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(1H-tetrazol-5-yl)isoxazole-3-carboxamide (1.10 g, 2.04 mmol) in a yield of 89%.

¹H-NMR (400 MHz, CDCl₃) δ 15.3 (br s, 1H), 7.44-7.36 (m, 6H), 7.29-7.24 (m, 3H), 6.98 (m, 2H), 6.65 (s, 1H), 5.11 (s, 2H), 4.88 (s, 2H), 3.56 (m, 2H), 3.35 (sept, 1H), 1.32 (t, 3H), 1.25 (d, 6H)

### Step 2

### 5-(2,4-Dihydroxy-5-isopropylphenyl)-N-ethyl-4-(1H-tetrazol-5-yl)isoxazole-3-carboxamide

This compound was made using the procedure described for example 1 (Step 3). Thus, this intermediate compound (Step 1) (20 mg, 0.04 mmol) was reacted with BCl₃ to afford a crude product, which was purified by silica gel column chromatography to afford the title compound (13 mg, 0.04 mmol) in a yield of 98%.

¹H-NMR (400 MHz, CD₃OD) δ 7.05 (s, 1H), 6.35 (s, 1H), 3.39 (q, 2H), 3.12 (sept, 1H), 1.21 (t, 3H), 1.10 (d, 6H)

### Example 11

### 5-(2,4-Dihydroxy-5-isopropylphenyl)-N-ethyl-4-(1-methyl-1H-tetrazol-5-yl)isox azole-3-carboxamide (I-11)

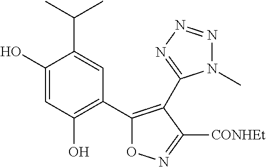

### Step 1

### 5-(2,4-Bis(benzyloxy)-5-isopropylphenyl)-N-ethyl-4-(1-methyl-1H-tetrazol-5-yl)isoxazole-3-carboxamide

To a suspension 5-(2,4-bis(benzyloxy)-5-isopropylphenyl)-N-ethyl-4-(1H-tetrazol-5-yl)isoxazole-3-carboxamide (900 mg, 1.67 mmol) in CH₃CN (20 ml) were added potassium carbonate (277 mg, 2.00 mmol) and iodomethane (0.12 ml, 2.00 mmol). And then the mixture was heated to reflux for 1 h, and iodomethane (0.12 ml, 2.00 mmol) was added. After 1 h, the reaciton mixture was cooled to RT, solvent was evaporated in vacuo, and the residue was extracted between ethyl acetate and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(1-methyl-1H-tetrazol-5-yl) isoxazole-3-carboxamide (410 mg, 0.74 mmol) in a yield of 44%.

¹H-NMR (400 MHz, CDCl₃) δ 7.53 (s, 1H), 7.34-7.31 (m, 8H), 7.09 (dd, 2H), 6.88 (br t, 1H), 6.39 (s, 1H), 4.98 (s, 2H), 4.58 (s, 2H), 3.47 (s, 3H), 3.44-3.37 (m, 2H), 3.30 (sept, 1H), 1.24-1.21 (m, 9H)

### Step 2

### 5-(2,4-Dihydroxy-5-isopropylphenyl)-N-ethyl-4-(1-methyl-1H-tetrazol-5-yl)isoxazole-3-carboxamide

This compound was made using the procedure described for example 1 (Step 3). Thus, this intermediate compound (Step 1) (410 mg, 0.74 mmol) was reacted with BCl₃ to afford a crude product, which was purified by silica gel column chromatography to afford the title compound (120 mg, 0.32 mmol) in a yield of 43%).

¹H-NMR (400 MHz, CD₃OD) δ 7.42 (s, 1H), 6.21 (s, 1H), 3.97 (s, 3H), 3.34 (q, 2H), 3.18 (sept, 1H), 1.21-1.16 (m, 9H)

### Example 12

### 5-(2,4-Dihydroxy-5-isopropylphenyl)-N-ethyl-4-(2-methyl-2H-tetrazol-5-yl)isoxazole-3-carboxamide (I-12)

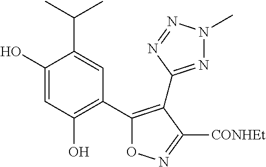

### Step 1

### 5-(2,4-Bis(benzyloxy)-5-isopropylphenyl)-N-ethyl-4-(2-methyl-2H-tetrazol-5-yl)isoxazole-3-carboxamide

This compound was made using the procedure described for example 11 (Step 1). Thus, 5-(2,4-bis(benzyloxy)-5-isopropylphenyl)-N-ethyl-4-(1H-tetrazol-5-yl)isoxazole-3-carboxamide (900 mg, 1.67 mmol) was reacted with iodomethane (0.12 ml, 2.00 mmol) to afford the intermediate compound 5-(2,4-bis(benzyloxy)-5-isopropylphenyl)-N-ethyl-4-(2-methyl-2H-tetrazol-5-yl) isoxazole-3-carboxamide (446 mg, 0.81 mmol) in a yield of 48%.

¹H-NMR (400 MHz, CDCl₃) δ 7.48 (s, 1H), 7.40-7.28 (m, 8H), 7.12 (m, 2H), 7.02 (br t, 1H), 6.43 (s, 1H), 4.99 (s, 2H), 4.81 (s, 2H), 4.15 (s, 3H), 3.48 (m, 2H), 3.29 (sept, 1H), 1.25 (t, 3H), 1.20 (d, 6H)

### Step 2

### 5-(2,4-Dihydroxy-5-isopropylphenyl)-N-ethyl-4-(2-methyl-2H-tetrazol-5-yl)isoxazole-3-carboxamide

This compound was made using the procedure described for example 1 (Step 3). Thus, this intermediate compound (Step 1) (446 mg, 0.81 mmol) was reacted with BCl₃ to afford a crude product, which was purified by silica gel column chromatography to afford the title compound (260 mg, 0.70 mmol) in a yield of 86%.

¹H-NMR (400 MHz, CD₃OD) δ 7.26 (s, 1H), 6.28 (s, 1H), 4.36 (s, 3H), 3.38 (q, 2H), 3.17 (sept, 1H), 1.22 (t, 3H), 1.17 (d, 6H)

### Example 13

### 5-(2,4-Dihydroxy-5-isopropylphenyl)-N-ethyl-4-(1-ethyl-1H-tetrazol-5-yl)isoxa zole-3-carboxamide (I-13)

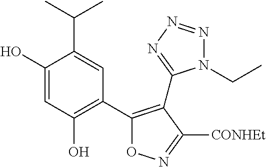

### Step 1

### 5-(2,4-Bis(benzyloxy)-5-isopropylphenyl)-N-ethyl-4-(1-ethyl-1H-tetrazol-5-isoxazole-3-carboxamide

This compound was made using the procedure described for example 11 (Step 1). Thus, 5-(2,4-bis(benzyloxy)-5-isopropylphenyl)-N-ethyl-4-(1H-tetrazol-5-yl) isoxazole-3-carboxamide (200 mg, 0.37 mmol) was reacted with iodoethane (35.6 μl, 0.44 mmol) to afford the intermediate compound 5-(2,4-bis(benzyloxy)-5-isopropylphenyl)-N-ethyl-4-(1-ethyl-1H-tetrazol-5-yl) isoxazole-3-carboxamide (60 mg, 0.10 mmol) in a yield of 28%.

¹H-NMR (400 MHz, CDCl₃) δ 7.46 (s, 1H), 7.38-7.26 (m, 8H), 7.09 (m, 2H), 6.89 (br t, 1H), 6.32 (s, 1H), 4.89 (s, 2H), 4.64 (s, 2H), 4.01 (q, 2H), 3.42 (m, 2H), 3.26 (sept, 1H), 1.42 (t, 3H), 1.22 (t, 3H), 1.19 (d, 6H)

### Step 2

### 5-(2,4-Dihydroxy-5-isopropylphenyl)-N-ethyl-4-(1-ethyl-1H-tetrazol-5-yl)isoxazole-3-carboxamide

This compound was made using the procedure described for example 1 (Step 3). Thus, this intermediate compound (Step 1) (57 mg, 0.10 mmol) was reacted with BCl₃ to afford a crude product, which was purified by silica gel column chromatography to afford the title compound (22 mg, 0.06 mmol) in a yield of 57%.

¹H-NMR (400 MHz, CD₃OD) δ 7.41 (s, 1H), 6.22 (s, 1H), 4.25 (q, 2H), 3.34 (m, 2H), 3.18 (sept, 1H), 1.52 (t, 3H), 1.20 (d, 6H), 1.18 (t, 3H)

### Example 14

### 5-(2,4-Dihydroxy-5-isopropylphenyl)-N-ethyl-4-(2-ethyl-2H-tetrazol-5-yl)isoxazole-3-carboxamide (I-14)

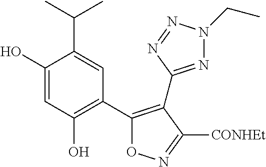

### Step 1

### 5-(2,4-Bis(benzyloxy)-5-isopropylphenyl)-N-ethyl-4-(2-ethyl-2H-tetrazol-5-yl) isoxazole-3-carboxamide

This compound was made using the procedure described for example 11 (Step 1).

Thus, 5-(2,4-bis(benzyloxy)-5-isopropylphenyl)-N-ethyl-4-(1H-tetrazol-5-yl)isoxazole-3-carboxamide (200 mg, 0.37 mmol) was reacted with iodoethane (35.6 μl, 0.44 mmol) to afford the intermediate compound 5-(2,4-bis(benzyloxy)-5-isopropylphenyl)-N-ethyl-4-(2-ethyl-2H-tetrazol-5-yl) isoxazole-3-carboxamide (140 mg, 0.25 mmol) in a yield of 66%.

¹H-NMR (400 MHz, CDCl₃) δ 7.44 (s, 1H), 7.40-7.26 (m, 8H), 7.13 (m, 2H), 7.10 (br t, 1H), 6.42 (s, 1H), 4.97 (s, 2H), 4.56 (s, 2H), 4.53 (q, 2H), 3.47 (m, 2H), 3.28 (sept, 1H), 1.54 (t, 3H), 1.25 (t, 3H), 1.18 (d, 6H)

### Step 2

### 5-(2,4-Dihydroxy-5-isopropylphenyl)-N-ethyl-4-(2-ethyl-2H-tetrazol-5-yl)isoxazole-3-carboxamide

This compound was made using the procedure described for example 1 (Step 3). Thus, this intermediate compound (Step 1) (135 mg, 0.24 mmol) was reacted with BCl₃ to afford a crude product, which was purified by silica gel column chromatography to afford the title compound (278 mg, 0.20 mmol) in a yield of 85%.

¹H-NMR (400 MHz, CD₃OD) δ 7.25 (s, 1H), 6.28 (s, 1H), 4.69 (q, 2H), 3.38 (q, 2H), 3.17 (sept, 1H), 1.59 (t, 3H), 1.22 (t, 3H), 1.17 (d, 6H)

### Example 15

### Ethyl 5-(2,4-dihydroxy-5-isopropylphenyl)-3-(ethylcarbamoyl)-4,5′-biisoxazole-3′-carboxylate (I-15)

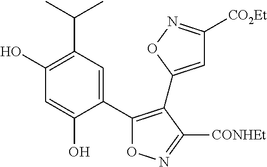

### Step 1

### Ethyl 5-(2,4-bis(benzyloxy)-5-isopropylphenyl)-3-(ethylcarbamoyl)-4,5′-biisoxazole-3′-carboxylate

This compound was made using the procedure described for example 7 (Step 1). Thus, 5-(2,4-bis(benzyloxy)-5-isopropylphenyl)-N-ethyl-4-iodoisoxazole-3-carboxamide (118 mg, 0.199 mmol) was reacted with tetrakis(triphenylphosphine)palladium(0) (11.5 mg, 0.01 mmol) and ethyl 5-(tributylstannyl)isoxazole-3-carboxylate (102.7 mg, 0.238 mmol) to afford the intermediate compound ethyl 5-(2,4-bis(benzyloxy)-5-isopropylphenyl)-3-(ethylcarbamoyl)-4,5′-biisoxazole-3′-carboxylate (94.8 mg, 0.155 mmol) in a yield of 78%.

¹H-NMR (400 MHz, CDCl₃) δ 7.42-7.24 (m, 9H), 7.09-7.04 (m, 2H), 6.88 (t, 1H), 6.53 (s, 1H), 5.04 (d, 2H), 4.79 (s, 2H), 4.40 (q, 2H), 3.48 (m, 2H), 3.33 (m, 1H), 1.40 (t, 3H), 1.27-1.23 (m, 9H)

### Step 2

### Ethyl 5-(2,4-dihydroxy-5-isopropylphenyl)-3-(ethylcarbamoyl)-4,5′-biisoxazole-3′-ca rboxylate

This compound was made using the procedure described for example 1 (Step 3). Thus, this intermediate compound (Step 1) was reacted with BCl₃ to afford a crude product, which was purified by silica gel column chromatography to afford the title compound (37 mg, 0.086 mmol) in a yield of 56%.

¹H-NMR (400 MHz, CDCl₃) δ 7.26 (s, 1H), 6.88 (s, 1H), 6.54 (s, 1H), 4.33 (q, 2H), 3.42 (m, 2H), 3.21 (m, 1H), 1.31-1.21 (m, 12H)

### Example 16

### 5-(2,4-Dihydroxy-5-isopropylphenyl)-N³,N^{3′}-diethyl-4,5′-biisoxazole-3,3′-dicarboxamide (I-16)

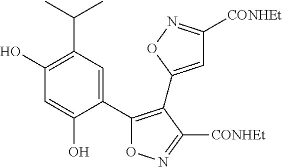

### Step 1

### 5-(2,4-Bis(benzyloxy)-5-isopropylphenyl)-N³,N^{3′}-diethyl-4,5′-biisoxazole-3,3′-dicarboxamide

2M-ethylamine solution (3.99 ml) was added to a ethyl 5-(2,4-bis(benzyloxy)-5-isopropylphenyl)-3-(ethylcarbamoyl)-4,5′-biisoxazole-3′-carboxylate (486.7 mg, 0.798 mmol) in EtOH (4.89 ml), and the reaction mixture was heated to reflux for 2 h. The mixture was cooled to ambient temperature, solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N³,N^{3′}-diethyl-4,5′-biisoxazole-3,3′-d icarboxamide (415.3 mg, 0.68 mmol) in a yield of 86%.

¹H-NMR (400 MHz, CDCl₃) δ 7.41-7.24 (m, 6H), 7.29-7.26 (m, 3H), 7.10-7.08 (m, 2H), 6.99 (s, 1H), 6.79 (s, 1H), 6.70 (s, 1H), 6.50 (s, 1H), 5.02 (s, 2H), 4.83 (s, 2H), 3.51-3.42 (m, 4H), 3.31 (m, 1H), 1.27-1.20 (m, 12H)

### Step 2

### 5-(2,4-Dihydroxy-5-isopropylphenyl)-N³,N^{3′}-diethyl-4,5′-biisoxazole-3,3′-dicarboxamide

This compound was made using the procedure described for example 1 (Step 3). Thus, this intermediate compound (Step 1) (410.6 mg, 0.675 mmol) was reacted with BCl₃ to afford a crude product, which was purified by silica gel column chromatography to afford the title compound (278.3 mg, 0.649 mmol) in a yield of 96%.

¹H-NMR (400 MHz, CDCl₃) δ 7.21 (s, 1H), 6.89 (s, 1H), 6.30 (s, 1H), 3.49 (m, 4H), 3.19 (m, 1H), 1.28-1.18 (m, 12H)

### Example 17

### 5-(2,4-Dihydroxy-5-isopropylphenyl)-N-ethyl-3′-(hydroxymethyl)-4,5′-biisoxazole-3-carboxamide (I-17)

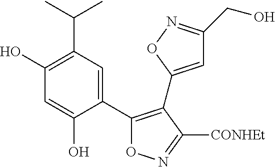

### Step 1

### 5-(2,4-Bis(benzyloxy)-5-isopropylphenyl)-N-ethyl-3′-(hydroxymethyl)-4,5′-biisoxazole-3-carboxamide To ethyl 5-(2,4-bis(benzyloxy)-5-isopropylphenyl)-3-(ethylcarbamoyl)-4,5′-biisoxazole-3′-carboxylate (828.4 mg, 1.35 mmol) in THF (13.5 ml) cooled to 0° C. under N₂ was added lithium aluminium hydride (77.32 mg, 2.03 mmol). The reaction was allowed to warm to RT and was stirred for 2 h. After this time, the reaction mixture was cooled to 0° C., and sequentially water (0.08 ml), a 10% NaOH solution (0.16 ml), and water (0.24 ml) were added. The reaction mixture was left to sitr at RT, diethylether (15 ml) added. After being stirred for 30 min, the reaction mixture was filtered through a pad of Celite, and the filtrate was concentrated. The residue was purified by silica gel column chromatography to afford the intermediate compound 5-(2,4-bis(benzyloxy)-5-isopropylphenyl)-N-ethyl-3′-(hydroxymethyl)-4,5′-biis oxazole-3-carboxamide (472.5 mg, 0.83 mmol) in a yield of 61%.

¹H-NMR (400 MHz, CDCl₃) δ 7.39-7.25 (m, 9H), 7.13-7.12 (m, 2H), 6.89 (s, 1H), 6.73 (s, 1H), 6.53 (s, 1H), 5.03 (s, 2H), 4.83 (s, 2H), 4.59 (s, 2H), 3.45 (q, 2H), 3.36-3.29 (m, 1H), 1.25-1.21 (m, 9H)

### Step 2

### 5-(2,4-Dihydroxy-5-isopropylphenyl)-N-ethyl-3′-(hydroxymethyl)-4,5′-biisoxazole-3-carboxamide

This compound was made using the procedure described for example 1 (Step 3). Thus, this intermediate compound (Step 1) (511.3 mg, 0.9 mmol) was reacted with BCl₃ to afford a crude product, which was purified by silica gel column chromatography to afford the title compound (322.6 mg, 0.83 mmol) in a yield of 93%.

¹H-NMR (400 MHz, CDCl₃) δ 7.13 (s, 1H), 6.56 (s, 1H), 6.38 (s, 1H), 4.61 (s, 2H), 3.42 (q, 2H), 3.18 (m, 1H), 1.23 (t, 3H), 1.18 (d, 6H)

### Example 18

### 5-(2,4-Dihydroxy-5-isopropylphenyl)-N-ethyl-3′-(morpholinomethyl)-4,5′-biisox azole-3-carboxamide (I-18)

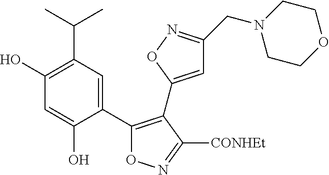

### Step 1

### (5-(2,4-Bis(benzyloxy)-5-isopropylphenyl)-3-(ethylcarbamoyl)-4,5′-biisoxazol-3′-yl)methyl methanesulfonate

To 5-(2,4-bis(benzyloxy)-5-isopropylphenyl)-N-ethyl-3′-(hydroxymethyl)-4,5′-biis oxazole-3-carboxamide (831.7 mg, 1.465 mmol) in methylene chloride (8 ml) cooled to 0° C. were added triethylamine (611.87 μl, 4.26 mmol) and methanesulfonyl chloride (226.7 μl, 2.93 mmol). The reaction mixture was left to stir at RT for 3 h, and extracted between methylene chloride and water. The organic phase was dried with magnesium sulfate, and evaporated in vacuo. The residue was purified by silica gel column chromatography to afford the intermediate compound

(5-(2,4-Bis(benzyloxy)-5-isopropylphenyl)-3-(ethylcarbamoyl)-4,5′-biisoxazol-3′-yl)methyl methanesulfonate (472.5 mg, 0.83 mmol) in a yield of 61%.

¹H-NMR (400 MHz, CDCl₃) δ 7.40-7.25 (m, 9H), 7.11 (m, 2H), 6.84 (s, 2H), 6.53 (s, 1H), 5.15 (s, 2H), 5.05 (s, 2H), 4.83 (s, 2H), 3.50 (m, 2H), 3.30 (m, 1H), 2.99 (s, 3H), 1.29-1.22 (m, 9H)

### Step 2

### 5-(2,4-Bis(benzyloxy)-5-isopropylphenyl)-N-ethyl-3′-(morpholinomethyl)-4,5′-biisoxazole-3-carboxamide

To a solution of the intermediate compound (Step 1) (866.4 mg, 1.34 mmol) in DMF (10.9 ml was added morpholine (458.4 μl, 5.36 mmol), and the reaction mixture was left to stir at RT for 2 h. After this time, solvent was evaporated in vacuo, and the residue was extracted between methylene chloride and water. The organic phase was dried with magnesium sulfate, and evaporated in vacuo. The residue was purified by silica gel column chromatography to afford the intermediate compound

5-(2,4-bis(benzyloxy)-5-isopropylphenyl)-N-ethyl-3′-(morpholinomethyl)-4,5′-biisoxazole-3-carboxamide (741.6 mg, 1.165 mmol) in a yield of 87%.

¹H-NMR (400 MHz, CDCl₃) δ 7.40-7.25 (m, 9H), 7.15-7.13 (m, 2H), 6.87 (s, 2H), 6.53 (s, 1H), 5.03 (s, 2H), 4.88 (s, 2H), 3.64 (t, 4H), 3.53-3.46 (m, 4H), 3.31 (m, 1H), 2.42 (t, 4H), 1.29-1.20 (m, 9H)

### Step 3

### 5-(2,4-Dihydroxy-5-isopropylphenyl)-N-ethyl-3′-(morpholinomethyl)-4,5′-biisoxazole-3-carboxamide

This compound was made using the procedure described for example 1 (Step 3). Thus, this intermediate compound (Step 2) (736.6 mg, 1.157 mmol) was reacted with BCl₃ to afford a crude product, which was purified by silica gel column chromatography to afford the title compound (547 mg, 1.19 mmol) in a yield of 99%.

¹H-NMR (400 MHz, CD₃OD) δ 7.14 (s, 1H), 6.53 (s, 1H), 6.37 (s, 1H), 3.69-3.67 (m, 4H), 3.59 (s, 2H), 3.41 (q, 2H), 3.19 (t, 1H), 2.50 (t, 4H), 1.28-1.17 (m, 9H)

### Example 19

### Methyl 2-((5-(2,4-dihydroxy-5-isopropylphenyl)-3-(ethylcarbamoyl)-4,5′-biisoxazol-3′-yl)methoxy)acetate (I-19)

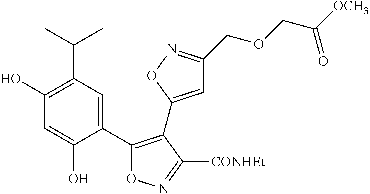

### Step 1

### 5-(2,4-Bis(benzyloxy)-5-isopropylphenyl)-3-(ethylcarbamoyl)-4,5′-biisoxazol-3′-yl)methoxy)acetate

To a solution of 5-(2,4-bis(benzyloxy)-5-isopropylphenyl)-N-ethyl-3′-(hydroxymethyl)-4,5′-biis oxazole-3-carboxamide (59.1 mg, 0.104 mmol) in CH₃CN (1 ml) were added methyl bromoacetate (12.5 μl, 0.135 mmol) and cesium carbonate (50.9 mg, 0.156 mmol), and the reaction mixture was left to stir at RT for overnight. After this time, solvent was evaporated in vacuo, and the residue was extracted between methylene chloride and water. The organic phase was dried with magnesium sulfate, and evaporated in vacuo to give the intermediate compound 5-(2,4-bis(benzyloxy)-5-isopropylphenyl)-3-(ethylcarbamoyl)-4,5′-biisoxazol-3′-yl)methoxy)acetate (20.7 mg, 0.032 mmol) in a yield of 61%.

¹H-NMR (400 MHz, CDCl₃) δ 7.42-7.26 (m, 9H), 7.14-7.12 (m, 2H), 6.83 (d, 2H), 6.53 (s, 1H), 5.03 (s, 2H), 4.85 (s, 2H), 4.62 (s, 2H), 4.07 (s, 2H), 3.74 (s, 3H), 3.49 (q, 2H), 3.32 (m, 1H), 1.29-1.21 (m, 9H)

### Step 2

### Methyl 2-((5-(2,4-Dihydroxy-5-isopropylphenyl)-3-(ethylcarbamoyl)-4,5′-biisoxazol-3′-yl)methoxy)acetate

This compound was made using the procedure described for example 1 (Step 3). Thus, this intermediate compound (Step 1) was reacted with BCl₃ to afford a crude product, which was purified by silica gel column chromatography to afford the title compound (10 mg, 0.0217 mmol) in a yield of 67%.

¹H-NMR (400 MHz, CD₃OD) δ 7.14 (s, 1H), 6.58 (s, 1H), 6.38 (s, 1H), 4.67 (s, 2H), 4.18 (s, 2H), 3.73 (s, 3H), 3.41 (m, 2H), 3.17 (m, 1H), 1.28-1.17 (m, 9H)

### Example 20

3′-((Diethylamino)methyl)-5-(2,4-dihydroxy-5-isopropylphenyl)-N-ethyl-4,5′-bi isoxazole-3-carboxamide (I-20)

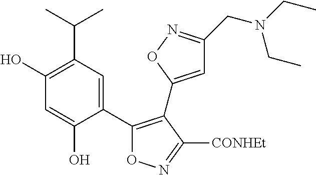

### Step 1

### 5-(2,4-Bis(benzyloxy)-5-isopropylphenyl)-3′-((diethylamino)methyl)-N-ethyl-4,5′-biisoxazole-3-carboxamide

This compound was made using the procedure described for example 18 (Step 2).

Thus, (5-(2,4-bis(benzyloxy)-5-isopropylphenyl)-3-(ethylcarbamoyl)-4,5′-biisoxazol-3′-yl)methyl methanesulfonate (38.1 mg, 0.059 mmol) was reacted with diethylamine (24.4 μl, 0.236 mmol) to afford the intermediate compound 5-(2,4-bis(benzyloxy)-5-isopropylphenyl)-3′-((diethylamino)methyl)-N-ethyl-4,5′-biisoxazole-3-carboxamide (26.2 mg, 0.042 mmol) in a yield of 71%.

¹H-NMR (400 MHz, CDCl₃) δ 7.41-7.25 (m, 9H), 7.17-7.15 (m, 2H), 6.83-6.79 (m, 2H), 6.52 (s, 1H), 5.02 (s, 2H), 4.88 (s, 2H), 3.65 (s, 2H), 3.49 (q, 2H), 3.31 (m, 1H), 2.49 (q, 4H), 1.29-1.19 (m, 12H), 1.03 (t, 3H)

### Step 2

### 3′-((Diethylamino)methyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (18 mg, 0.04 mmol) in a yield of 99%.

¹H-NMR (400 MHz, CD₃OD) δ 7.13 (s, 1H), 6.56 (s, 1H), 6.41 (s, 1H), 3.84 (s, 2H), 3.42 (m, 2H), 3.19 (m, 1H), 2.68 (m, 4H), 1.26 (t, 3H), 1.18-1.12 (m, 12H)

### Example 21

### 5-(2,4-Dihydroxy-5-isopropylphenyl)-N³-ethyl-4,5′-biisoxazole-3,3′-dicarboxamide (I-21)

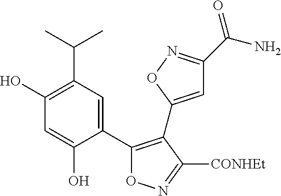

### Step 1

### 5-(2,4-Bis(benzyloxy)-5-isopropylphenyl)-N³-ethyl-4,5′-biisoxazole-3,3′-dicarboxamide

To a solution of ethyl 5-(2,4-bis(benzyloxy)-5-isopropylphenyl)-3-(ethylcarbamoyl)-4,5′-biisoxazole-3′-carboxylate (53 mg, 0.087 mmol) in 7N—NH₃/MeOH (1.45 ml) was added potassium cyanide (KCN) (1.4 mg, 0.022 mmol). The reaction mixture was heated for overnight at 50° C., and then cooled to ambient temperature, solvent was evaporated in vacuo to give the intermediate compound 5-(2,4-bis(benzyloxy)-5-isopropylphenyl)-N³-ethyl-4,5′-biisoxazole-3,3′-dicarb oxamide (45.8 mg, 0.078 mmol) in a yield of 91%.

¹H-NMR (400 MHz, CDCl₃) δ 7.41-7.32 (m, 6H), 7.30-7.26 (m, 3H), 7.10-7.08 (m, 2H), 7.02 (s, 1H), 6.85 (s, 1H), 6.69 (s, 1H), 6.52 (s, 1H), 5.93 (s, 1H), 5.03 (s, 2H), 4.82 (s, 2H), 3.47 (q, 2H), 3.32 (m, 1H), 1.29-1.21 (m, 9H)

### Step 2

### 5-(2,4-Dihydroxy-5-isopropylphenyl)-N³-ethyl-4,5′-biisoxazole-3,3′-dicarboxamide

This compound was made using the procedure described for example 1 (Step 3). Thus, this intermediate compound (Step 1) was reacted with BCl₃ to afford a crude product, which was purified by silica gel column chromatography to afford the title compound (30.8 mg, 0.077 mmol) in a yield of 99%.

¹H-NMR (400 MHz, CD₃OD) δ 7.19 (s, 1H), 6.84 (s, 1H), 6.38 (s, 1H), 3.42 (m, 2H), 3.20 (m, 1H), 1.28-1.18 (m, 9H)

### Example 22

### 5-(2,4-Dihydroxy-5-isopropylphenyl)-N-ethyl-3′-((2-hydroxyethylamino)methyl)-4,5′-biisoxazole-3-carboxamide (I-22)

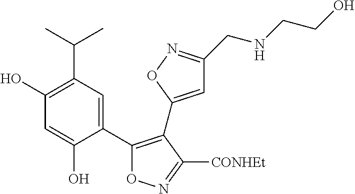

### Step 1

### 5-(2,4-Bis(benzyloxy)-5-isopropylphenyl)-N-ethyl-3′-((2-hydroxyethylamino)methyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (50 mg, 0.077 mmol) was reacted with hydroxyethylamine (18.7 μl, 0.309 mmol) to afford the intermediate compound 5-(2,4-bis(benzyloxy)-5-isopropylphenyl)-N-ethyl-3′-((2-hydroxyethylamino)methyl)-4,5′-biisoxazole-3-carboxamide (42.6 mg, 0.0697 mmol) in a yield of 91%.

¹H-NMR (400 MHz, CDCl₃) δ 7.42-7.24 (m, 9H), 7.15-7.13 (m, 2H), 6.77 (s, 1H), 6.51 (s, 1H), 5.11 (s, 2H), 4.93 (s, 2H), 3.74 (s, 2H), 3.60 (t, 2H), 3.40 (q, 2H), 3.32-3.30 (m, 2H), 2.66 (t, 2H), 1.27-1.19 (m, 9H)

### Step 2

### 5-(2,4-Dihydroxy-5-isopropylphenyl)-N-ethyl-3′-((2-hydroxyethylamino)methyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (29.4 mg, 0.068 mmol) in a yield of 98%.

¹H-NMR (400 MHz, CD₃OD) δ 7.15 (s, 1H), 6.58 (s, 1H), 6.39 (s, 1H), 3.97 (s, 2H), 3.69 (t, 2H), 3.42 (q, 2H), 3.19 (m, 1H), 2.84 (t, 2H), 1.25-1.17 (m, 9H)

### Example 23

### 5-(2,4-Dihydroxy-5-isopropylphenyl)-N-ethyl-3′-(morpholine-4-carbonyl)-4,5′-biisoxazole-3-carboxamide (I-23)

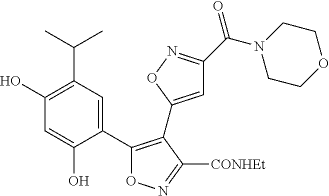

### Step 1

### 5-(2,4-Bis(benzyloxy)-5-isopropylphenyl)-N-ethyl-3′-(morpholine-4-carbonyl)-4,5′-biisoxazole-3-carboxamide

This compound was made using the procedure described for example 16 (Step 1). Thus, ethyl 5-(2,4-bis(benzyloxy)-5-isopropylphenyl)-3-(ethylcarbamoyl)-4,5′-biisoxazole-3′-carboxylate (51.5 mg, 0.0845 mmol) was reacted with morpholine (72.13 μl, 0.845 mmol) to afford the intermediate compound 5-(2,4-bis(benzyloxy)-5-isopropylphenyl)-N-ethyl-3′-(morpholine-4-carbonyl)-4,5′-biisoxazole-3-carboxamide (32.6 mg, 0.05 mmol,) in a yield of 86%.

¹H-NMR (400 MHz, CDCl₃) δ 7.39-7.26 (m, 9H), 7.13-7.11 (m, 2H), 7.03 (s, 1H), 6.83 (s, 1H), 6.52 (s, 1H), 5.01 (s, 2H), 4.86 (s, 2H), 3.78-3.76 (m, 6H), 3.65-3.63 (m, 2H), 3.49 (q, 2H), 3.31 (m, 1H), 1.29-1.20 (m, 9H)

### Step 2

### 5-(2,4-Dihydroxy-5-isopropylphenyl)-N-ethyl-3′-(morpholine-4-carbonyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (15.2 mg, 0.032 mmol) in a yield of 65%.

¹H-NMR (400 MHz, CD₃OD) δ 7.20 (s, 1H), 6.77 (s, 1H), 6.36 (s, 1H), 3.82-3.78 (m, 6H), 3.72-3.70 (m, 2H), 3.44 (q, 2H), 3.20 (m, 1H), 1.26 (t, 3H), 1.19 (d, 6H)

### Example 24

### 5-(5-Chloro-2,4-dihydroxyphenyl)-N-ethyl-3′-(hydroxymethyl)-4,5′-biisoxazole-3-carboxamide (I-24)

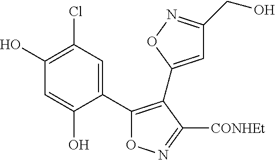

### Step 1

### 5-(2,4-Bis(benzyloxy)-5-chlorophenyl)-N-ethyl-3′-(hydroxymethyl)-4,5′-biisoxazole-3-carboxamide

This compound was made using the procedure described for example 17 (Step 1). Thus, ethyl 5-(2,4-bis(benzyloxy)-5-chlorophenyl)-3-(ethylcarbamoyl)-4,5′-biisoxazole-3′-carboxylate (71.8 mg, 0.12 mmol) was reacted with lithium aluminium hydride (6.78 mg, 0.178 mmol) to afford the intermediate compound 5-(2,4-bis(benzyloxy)-5-chlorophenyl)-N-ethyl-3′-(hydroxymethyl)-4,5′-biisoxa zole-3-carboxamide (17 mg, 0.03 mmol) in a yield of 25%.

¹H-NMR (400 MHz, CDCl₃) δ 7.59 (s, 1H), 7.40-7.26 (m, 9H), 7.09-7.07 (m, 2H), 6.87-6.80 (m, 2H), 6.59 (s, 1H), 5.12 (s, 2H), 4.80 (s, 2H), 4.61 (s, 2H), 3.47 (q, 2H), 1.26 (t, 3H)

### Step 2

### 5-(5-Chloro-2,4-dihydroxyphenyl)-N-ethyl-3′-(hydroxymethyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (8 mg, 0.02 mmol) in a yield of 70%.

¹H-NMR (400 MHz, CD₃OD) δ 7.36 (s, 1H), 6.62 (s, 1H), 6.52 (s, 1H), 4.62 (s, 2H), 3.42 (q, 2H), 1.23 (t, 3H)

### Example 25

### Ethyl 5-(5-chloro-2,4-dihydroxyphenyl)-3-(ethylcarbamoyl)-4,5′-biisoxazole-3′-carbo xylate (I-25)

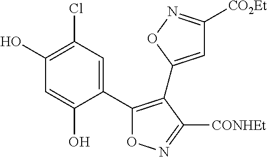

This compound was made using the procedure described for example 1 (Step 3). Thus, this compound ethyl 5-(2,4-bis(benzyloxy)-5-chlorophenyl)-3-(ethylcarbamoyl)-4,5′-biisoxazole-3′-carboxylate (21.3 mg, 0.0353 mmol) was reacted with BCl₃ to afford a crude product, which was purified by silica gel column chromatography to afford the title compound (7.2 mg, 0.017 mmol) in a yield of 48%.

¹H-NMR (400 MHz, CD₃OD) δ 7.44 (s, 1H), 6.97 (s, 1H), 6.50 (s, 1H), 4.41 (q, 2H), 3.42 (m, 2H), 1.38 (t, 3H), 1.23 (t, 3H)

### Example 26

### 5-(2,4-Dihydroxy-5-isopropylphenyl)-3-(ethylcarbamoyl)-4,5′-biisoxazole-3′-carboxylic acid (I-26)

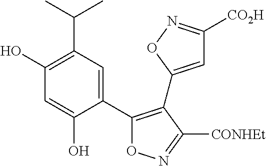

### Step 1

### 5-(2,4-Bis(benzyloxy)-5-isopropylphenyl)-3-(ethylcarbamoyl)-4,5′-biisoxazole-3′-carboxylic acid

Ethyl 5-(2,4-bis(benzyloxy)-5-isopropylphenyl)-3-(ethylcarbamoyl)-4,5′-biisoxazole-3′-carboxylate (97.5 mg, 0.16 mmol) was dissolved in THF/water (2.5 ml/1.65 ml), and this solution was cooled to 0° C. 2N—LiOH (99.7 μl) was added dropwise, and the reaction mixture was stirred at same condition for 1 h. 2N—HCl was added to acidify (pH 4) the reaciton mixture.

Solvents were removed in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-3-(ethylcarbamoyl)-4,5′-biisoxazole-3′-carboxylic acid (86.1 mg, 0.148 mmol) in a yield of 93%.

¹H-NMR (400 MHz, CDCl₃) δ 7.40-7.30 (m, 6H), 7.22-7.18 (m, 3H), 7.09-7.07 (m, 2H), 6.75 (s, 1H), 6.69 (s, 1H), 5.08 (s, 2H), 4.86 (s, 2H), 3.38 (q, 2H), 3.31 (m, 1H), 1.27-1.17 (m, 9H)

### Step 2

### 5-(2,4-Dihydroxy-5-isopropylphenyl)-3-(ethylcarbamoyl)-4,5′-biisoxazole-3′-carboxylic acid

This compound was made using the procedure described for example 1 (Step 3). Thus, this intermediate compound (Step 1) (42.1 mg, 0.0723 mmol) was reacted with BCl₃ to afford a crude product, which was purified by silica gel column chromatography to afford the title compound (27 mg, 0.68 mmol) in a yield of 94%.

¹H-NMR (400 MHz, DMSO-d₆) δ 10.10 (br, 1H), 9.97 (br, 1H), 8.98 (t, 1H), 7.05 (s, 1H), 6.57 (s, 1H), 6.46 (s, 1H), 3.31-3.26 (m, 2H), 3.09 (m, 1H), 1.13-1.10 (m, 9H)

### Example 27

### 5-(2,4-Dihydroxy-5-isopropylphenyl)-N-ethyl-3′-((ethylamino)methyl)-4,5′-biisoxazole-3-carboxamide (I-27)

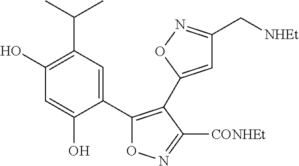

### Step 1

### 5-(2,4-Bis(benzyloxy)-5-isopropylphenyl)-N-ethyl-3′-((ethylamino)methyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (52.2 mg, 0.081 mmol) was reacted with 2N-ethylamine (161.7 μl, 0.323 mmol) to afford the intermediate compound 5-(2,4-bis(benzyloxy)-5-isopropylphenyl)-N-ethyl-3′-((ethylamino)methyl)-4,5′-biisoxazole-3-carboxamide (38.8 mg, 0.065 mmol) in a yield of 81%.

¹H-NMR (400 MHz, CDCl₃) δ 7.43-7.26 (m, 9H), 7.16-7.14 (m, 2H), 6.78 (s, 1H), 6.51 (s, 1H), 5.13 (s, 2H), 4.94 (s, 2H), 3.71 (s, 2H), 3.41 (q, 2H), 3.31-3.30 (m, 1H), 2.57 (q, 2H), 1.24-1.20 (m, 9H), 1.07 (t, 3H)

### Step 2

### 5-(2,4-Dihydroxy-5-isopropylphenyl)-N-ethyl-3′-((ethylamino)methyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (27.0 mg, 0.064 mmol) in a yield of 98%.

¹H-NMR (400 MHz, DMSO-d₆) δ 9.99 (br, 1H), 9.87 (br, 1H), 8.98 (t, 1H), 7.05 (s, 1H), 6.52 (s, 1H), 6.51 (s, 1H), 3.70 (s, 2H), 3.30-3.25 (m, 2H), 3.09 (m, 1H), 2.53-2.47 (m, 2H), 1.14-1.10 (m, 9H), 0.99 (t, 3H)

### Example 28

### 5-(2,4-Dihydroxy-5-isopropylphenyl)-N-ethyl-3′-(fluoromethyl)-4,5′-biisoxazole-3-carboxamide (I-28)

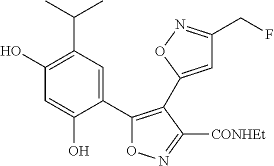

### Step 1

### 5-(2,4-Bis(benzyloxy)-5-isopropylphenyl)-N-ethyl-3′-(fluoromethyl)-4,5′-biisoxazole-3-carboxamide

(5-(2,4-Bis(benzyloxy)-5-isopropylphenyl)-3-(ethylcarbamoyl)-4,5′-biisoxazol-3′-yl)methyl methanesulfonate (50 mg, 0.077 mmol) was dissolved in CH₃CN (1 ml), potassium fluoride (KF) (8.99 mg, 0.154 mmol) and 18-crown-6 (2 mg, 0.0077 mmol) were added, and the reaction mixture was stirred at RT for 24 h. Solvent was evaporated in vacuo, and the residue was purified by silica gel column chromatography to afford the intermediate compound 5-(2,4-bis(benzyloxy)-5-isopropylphenyl)-N-ethyl-3′-(fluoromethyl)-4,5′-biiso xazole-3-carboxamide (34.2 mg, 0.06 mmol) in a yield of 78%.

¹H-NMR (400 MHz, CDCl₃) δ 7.38-7.254 (m, 9H), 7.12-7.10 (m, 2H), 6.88-6.83 (m, 2H), 6.53 (s, 1H), 5.39 (s, 1H), 5.28 (s, 1H), 5.02 (s, 2H), 4.82 (s, 2H), 3.48 (m, 2H), 3.32 (m, 1H), 1.28-1.19 (m, 9H)

### Step 2

### 5-(2,4-Dihydroxy-5-isopropylphenyl)-N-ethyl-3′-(fluoromethyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (15.0 mg, 0.038 mmol) in a yield of 64%.

¹H-NMR (400 MHz, CD₃OD) δ 7.15 (s, 1H), 6.65 (s, 1H), 6.38 (s, 1H), 5.49 (s, 1H), 5.37 (s, 1H), 3.42 (q, 2H), 3.19 (m, 1H), 1.24 (t, 3H), 1.18 (d, 6H)

### Example 29

### 5-(5-(2,4-Dihydroxy-5-isopropylphenyl)-3-(ethylcarbamoyl)-1H-pyrazol-4-yl)-N-ethylisoxazole-3-carboxamide (I-29)

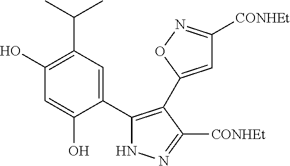

### Step 1

### Ethyl 5-(5-(2,4-bis(benzyloxy)-5-isopropylphenyl)-3-(ethylcarbamoyl)-1H-pyrazol-4-yl)isoxazole-3-carboxylate

This compound was made using the procedure described for example 7 (Step 1). Thus, 5-(2,4-bis(benzyloxy)-5-isopropylphenyl)-N-ethyl-4-iodo-1H-pyrazole-3-carboxamide (79 mg, 0.13 mmol) was reacted with ethyl 5-(tributylstannyl)isoxazole-3-carboxylate (62.77 mg, 0.145 mmol) to afford the intermediate compound ethyl 5-(5-(2,4-bis(benzyloxy)-5-isopropylphenyl)-3-(ethylcarbamoyl)-1H-pyrazol-4-yl)isoxazole-3-carboxylate (47 mg, 0.077 mmol) in a yield of 59%.

¹H-NMR (400 MHz, CDCl₃) δ 7.42-7.24 (m, 9H), 7.09-7.04 (m, 2H), 6.88 (t, 1H), 6.53 (s, 1H), 5.04 (d, 2H), 4.79 (s, 2H), 4.40 (q, 2H), 3.48 (m, 2H), 3.33 (m, 1H), 1.40 (t, 3H), 1.27-1.23 (m, 9H)

### Step 2

### 5-(5-(2,4-Bis(benzyloxy)-5-isopropylphenyl)-3-(ethylcarbamoyl)-1H-pyrazol-4-yl)-N-ethylisoxazole-3-carboxamide

This compound was made using the procedure described for example 18 (Step 2). Thus, this intermediate compound (Step 1) was reacted with 2M-ethylamine (0.386 ml) to afford the intermediate compound 5-(5-(2,4-bis(benzyloxy)-5-isopropylphenyl)-3-(ethylcarbamoyl)-1H-pyrazol-4-yl)-N-ethylisoxazole-3-carboxamide (14.3 mg, 0.023 mmol) in a yield of 30%.

¹H-NMR (400 MHz, CDCl₃) δ 7.42-7.30 (m, 10H), 6.93-6.91 (m, 3H), 6.80 (s, 1H), 6.61 (s, 1H), 5.06 (s, 4H), 3.50-3.37 (m, 4H), 3.21 (m, 1H), 1.29-1.18 (m, 6H), 1.01 (d, 6H)

### Step 3

### 5-(5-(2,4-Dihydroxy-5-isopropylphenyl)-3-(ethylcarbamoyl)-1H-pyrazol-4-yl)-N-ethylisoxazole-3-carboxamide

This compound was made using the procedure described for example 1 (Step 3). Thus, this intermediate compound (Step 2) was reacted with BCl₃ to afford a crude product, which was purified by silica gel column chromatography to afford the title compound (6 mg, 0.014 mmol) in a yield of 61%.

¹H-NMR (400 MHz, CD₃OD) δ 6.83 (s, 1H), 6.75 (s, 1H), 6.39 (s, 1H), 3.42-3.35 (m, 4H), 3.12 (m, 1H), 1.21 (q, 6H), 1.07 (d, 6H)

### Example 30

### 3′-(Aminomethyl)-5-(2,4-dihydroxy-5-isopropylphenyl)-N-ethyl-4,5′-biisoxazole-3-carboxamide (I-30)

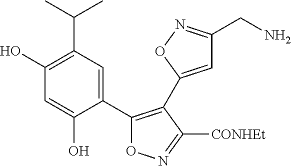

### Step 1

### 5-(2,4-Bis(benzyloxy)-5-isopropylphenyl)-3′-((1,3-dioxoisoindolin-2-yl)methyl)-N-ethyl-4,5′-biisoxazole-3-carboxamide

(5-(2,4-Bis(benzyloxy)-5-isopropylphenyl)-3-(ethylcarbamoyl)-4,5′-biisoxazol-3′-yl)methyl methanesulfonate (43.7 mg, 0.068 mmol) was dissolved in CH₃CN (1 ml), phtalimide potassium salt (37.6 mg, 0.203 mmol) was added, and the reaction mixture was heated to reflux for 24 h. The mixture was cooled to ambient temperature, solvent was evaporated in vacuo. The residue was filtered by solid impurities and the filtrate was evaporated in vacuo. The residue was purified by silica gel column chromatography to afford the intermediate compound 5-(2,4-bis(benzyloxy)-5-isopropylphenyl)-3′-((1,3-dioxoisoindolin-2-yl)methyl)-N-ethyl-4,5′-biisoxazole-3-carboxamide (44.8 mg, 0.0642 mmol) in a yield of 95%.

¹H-NMR (400 MHz, CDCl₃) δ 7.84 (m, 2H), 7.71 (m, 2H), 7.39-7.12 (m, 9H), 7.14-7.12 (m, 2H), 6.81 (s, 1H), 6.58 (s, 1H), 4.98 (s, 2H), 4.87 (s, 2H), 4.79 (2H), 3.46 (m, 2H), 3.27 (m, 1H), 1.27-1.17 (m, 9H)

### Step 2

### 3′-(Aminomethyl)-5-(2,4-bis(benzyloxy)-5-isopropylphenyl)-N-ethyl-4,5′-biisoxazole-3-carboxamide

The intermediate compound (Step 1) was dissolved in EtOH (0.74 ml), methylamine (40% w/w aqueous solution) (74.4 μl) was added. The reaction mixture was heated to reflux for 5.5 h, and then methylamine (40% w/w aqueous solution) (12.4 μl) was added. And then the reaction mixture was heated to reflux. After 1 h, the mixture was cooled to ambient temperature, solvent was evaporated in vacuo, and the residue was purified by silica gel column chromatography to afford the intermediate compound 3′-(aminomethyl)-5-(2,4-bis(benzyloxy)-5-isopropylphenyl)-N-ethyl-4,5′-biisox azole-3-carboxamide (41.9 mg, 0.074 mmol) in a yield of 99%.

¹H-NMR (400 MHz, CDCl₃) δ 7.43-7.26 (m, 9H), 7.16-7.14 (m, 2H), 6.78 (s, 1H), 6.48 (s, 1H), 5.13 (s, 2H), 4.93 (s, 2H), 3.70 (2H), 3.39 (q, 2H), 3.30 (m, 1H), 1.24-1.18 (m, 9H)

### Step 3

### 3′-(Aminomethyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 2) (20.7 mg, 0.0365 mmol) was reacted with BCl₃ to afford a crude product, which was purified by silica gel column chromatography to afford the title compound (16.1 mg, 0.04 mmol) in a yield of 99%.

¹H-NMR (400 MHz, CD₃OD) δ 7.14 (s, 1H), 6.58 (s, 1H), 6.39 (s, 1H), 4.01 (s, 2H), 3.45-3.39 (m, 2H), 3.18 (m, 1H), 1.28-1.17 (m, 9H)

### Example 31

### 3′-(Acetamidomethyl)-5-(2,4-dihydroxy-5-isopropylphenyl)-N-ethyl-4,5′-biisoxazole-3-carboxamide (I-31)

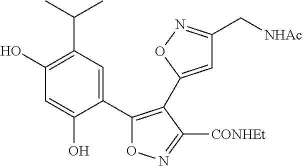

### Step 1

### 3′-(Acetamidomethyl)-5-(2,4-bis(benzyloxy)-5-isopropylphenyl)-N-ethyl-4,5′-biisoxazole-3-carboxamide

3′-(Aminomethyl)-5-(2,4-bis(benzyloxy)-5-isopropylphenyl)-N-ethyl-4,5′-biisoxazole-3-carboxamide (21.5 mg, 0.038 mmol) was dissolved in methylene chloride (1 ml), and this solution was cooled to 0° C. Acetic anhydride (7 μl) and pyridine (12.3 μl) were added, the resulting mixture was stirred at 0° C. for 30 min. And the residue was extracted between methylene chloride and water. The organic phase was dried with magnesium sulfate, and evaporated in vacuo. The residue was purified by silica gel column chromatography to afford the intermediate compound 3′-(acetamidomethyl)-5-(2,4-bis(benzyloxy)-5-isopropylphenyl)-N-ethyl-4,5′-biisoxazole-3-carboxamide (14 mg, 0.023 mmol) in a yield of 60%.

¹H-NMR (400 MHz, CDCl₃) δ 7.42-7.26 (m, 9H), 7.14-6.85 (m, 2H), 6.83 (t, 1H), 6.62 (s, 1H), 5.86 (s, 1H), 5.05 (s, 2H), 4.84 (s, 2H), 4.39 (d, 2H), 3.48 (q, 2H), 3.33 (m, 1H), 1.99 (s, 3H), 1.29-1.22 (m, 9H)

### Step 2

### 3′-(Acetamidomethyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (7.2 mg, 0.016 mmol) in a yield of 73%.

¹H-NMR (400 MHz, CD₃OD) δ 7.12 (s, 1H), 6.46 (s, 1H), 6.37 (s, 1H), 4.39 (s, 2H), 3.41 (q, 2H), 3.18 (m, 1H), 1.97 (s, 3H), 1.26-1.21 (m, 3H), 1.17 (d, 6H)

### Example 32

### (5-(2,4-Dihydroxy-5-isopropylphenyl)-3-(ethylcarbamoyl)-4,5′-biisoxazol-3′-yl) methyl methanesulfonate (I-32)

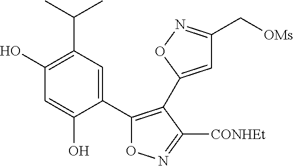

This compound was made using the procedure described for example 1 (Step 3). Thus, this compound(5-(2,4-bis(benzyloxy)-5-isopropylphenyl)-3-(ethylcarbamoyl)-4,5′-biisoxazol-3′-yl)methyl methanesulfonate (20.2 mg, 0.03 mmol) was reacted with BCl₃ to afford a crude product, which was purified by silica gel column chromatography to afford the title compound (17.1 mg, 0.036 mmol) in a yield of 99%.

¹H-NMR (400 MHz, CD₃OD) δ 7.17 (s, 1H), 6.68 (s, 1H), 6.37 (s, 1H), 5.32 (s, 2H), 3.41 (q, 2H), 3.20 (m, 1H), 3.11 (s, 3H), 1.23 (t, 3H), 1.18 (d, 6H)

### Example 33

### 2-((5-(2,4-Dihydroxy-5-isopropylphenyl)-3-(ethylcarbamoyl)-4,5′-biisoxazol-3′-yl)methoxy)acetic acid (I-33)

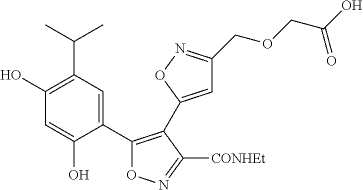

### Step 1

### 2-((5-(2,4-Bis(benzyloxy)-5-isopropylphenyl)-3-(ethylcarbamoyl)-4,5′-biisoxazol-3′-yl)methoxy)acetic acid

This compound was made using the procedure described for example 26 (Step 1). Thus, methyl 2-((5-(2,4-bis(benzyloxy)-5-isopropylphenyl)-3-(ethylcarbamoyl)-4,5-biisoxazol-3′-yl)methoxy)acetate (24.3 mg, 0.038 mmol) was reacted with 2N—LiOH (23.8 μl) to afford the intermediate compound 2-((5-(2,4-bis(benzyloxy)-5-isopropylphenyl)-3-(ethylcarbamoyl)-4,5′-biisoxazol-3′-yl)methoxy)acetic acid (15 mg, 0.024 mmol) in a yield of 63%.

¹H-NMR (400 MHz, CDCl₃) δ 7.35-7.13 (m, 9H), 7.00-6.98 (m, 2H), 6.69 (s 1H), 6.26 (s, 1H), 5.05 (s, 2H), 4.74 (s, 2H), 4.34 (s, 2H), 3.83 (s, 2H), 3.30-3.20 (m, 3H), 1.20-1.07 (m, 9H)

### Step 2

### 2-((5-(2,4-Dihydroxy-5-isopropylphenyl)-3-(ethylcarbamoyl)-4,5′-biisoxazol-3′-yl)methoxy)acetic acid

This compound was made using the procedure described for example 1 (Step 3). Thus, this intermediate compound (Step 1) was reacted with BCl₃ to afford a crude product, which was purified by silica gel column chromatography to afford the title compound (3.5 mg, 0.008 mmol) in a yield of 33%.

¹H-NMR (400 MHz, CD₃OD) δ 7.08 (s, 1H), 6.45 (s, 1H), 6.27 (s, 1H), 4.58 (s, 2H), 3.87 (s, 2H), 3.31 (q, 2H), 3.09 (m, 1H), 1.19-1.05 (m, 9H)

### Example 34

### 5-(2,4-Dihydroxy-5-isopropylphenyl)-N-ethyl-3′-(propionamidomethyl)-4,5′-biisoxazole-3-carboxamide (I-34)

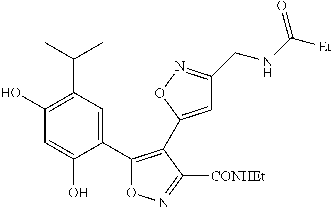

### Step 1

### 5-(2,4-Bis(benzyloxy)-5-isopropylphenyl)-N-ethyl-3′-(propionamidomethyl)-4,5′-biisoxazole-3-carboxamide

This compound was made using the procedure described for example 31 (Step 1). Thus, 3′-(aminomethyl)-5-(2,4-bis(benzyloxy)-5-isopropylphenyl)-N-ethyl-4,5′-biisoxazole-3-carboxamide (32.5 mg, 0.057 mmol) was reacted with propionic anhydride (11 μl, 0.086 mmol) to afford the intermediate compound 5-(2,4-bis(benzyloxy)-5-isopropylphenyl)-N-ethyl-3′-(propionamidomethyl)-4,5′-biisoxazole-3-carboxamide (33.9 mg, 0.0544 mmol) in a yield of 95%.

¹H-NMR (400 MHz, CDCl₃) δ 7.40-7.26 (m, 9H), 7.14-7.11 (m, 2H), 6.87 (s, 1H), 6.61 (s, 1H), 6.53 (s, 1H), 5.96 (s, 1H), 5.04 (s, 2H), 4.83 (s, 2H), 4.40 (d, 2H), 3.47 (q, 2H), 3.32 (m, 1H), 2.22 (q, 2H), 1.27-1.13 (m, 12H)

### Step 2

### 5-(2,4-Dihydroxy-5-isopropylphenyl)-N-ethyl-3′-(propionamidomethyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (10.4 mg, 0.024 mmol) in a yield of 45%

¹H-NMR (400 MHz, CD₃OD) δ 7.12 (s, 1H), 6.44 (s, 1H), 6.37 (s, 1H), 4.40-4.39 (m, 2H), 3.45-3.78 (m, 2H), 3.18 (m, 1H), 2.24 (q, 2H), 1.25-1.10 (m, 12H)

### Example 35

### 3′-(Cyanomethyl)-5-(2,4-dihydroxy-5-isopropylphenyl)-N-ethyl-4,5′-biisoxazole-3-carboxamide (I-35)

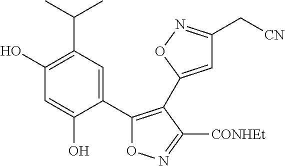

### Step 1

### 5-(2,4-Bis(benzyloxy)-5-isopropylphenyl)-3′-(cyanomethyl)-N-ethyl-4,5′-biisoxazole-3-carboxamide

This compound was made using the procedure described for example 28 (Step 1). Thus, (5-(2,4-bis(benzyloxy)-5-isopropylphenyl)-3-(ethylcarbamoyl)-4,5′-biisoxazol-3′-yl)methyl methanesulfonate (55.1 mg, 0.085 mmol) was reacted with potassium cyanide (KCN) (22.2 mg, 0.034 mmol) and 18-crown-6 (4.5 mg, 0.017 mmol) to afford the intermediate compound 5-(2,4-bis(benzyloxy)-5-isopropylphenyl)-3′-(cyanomethyl)-N-ethyl-4,5′-biisox azole-3-carboxamide (48.1 mg, 0.083 mmol) in a yield of 98%.

¹H-NMR (400 MHz, CDCl₃) δ 7.41-7.26 (m, 9H), 7.14-7.12 (m, 2H), 6.87 (s, 1H), 6.77 (s, 1H), 6.55 (s, 1H), 5.06 (s, 2H), 4.83 (s, 2H), 3.59 (s, 2H), 3.47 (q, 2H), 3.33 (m, 1H), 1.28-1.23 (m, 9H)

### Step 2

### 3′-(Cyanomethyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (15.0 mg, 0.037 mmol) in a yield of 45%.

¹H-NMR (400 MHz, CD₃OD) δ 7.16 (s, 1H), 6.60 (s, 1H), 6.38 (s, 1H), 4.01 (s, 2H), 3.45-3.38 (m, 2H), 3.18 (m, 1H), 1.23 (t, 3H), 1.19-1.16 (m, 6H)

### Example 36

### 3′-((2-Amino-2-oxoethoxy)methyl)-5-(2,4-dihydroxy-5-isopropylphenyl)-N-ethyl-4,5′-biisoxazole-3-carboxamide (I-36)

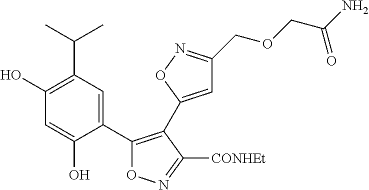

### Step 1

### 3′-((2-Amino-2-oxoethoxy)methyl)-5-(2,4-bis(benzyloxy)-5-isopropylphenyl)-N-ethyl-4,5′-biisoxazole-3-carboxamide

This compound was made using the procedure described for example 21 (Step 1). Thus, methyl 2-((5-(2,4-dihydroxy-5-isopropylphenyl)-3-(ethylcarbamoyl)-4,5′-biisoxazol-3′-yl)methoxy)acetate (34 mg, 0.053 mmol) was reacted with potassium cyanide (KCN) (0.86 mg, 0.25 mmol) and 7N—NH₃/MeOH (0.89 ml) to afford the intermediate compound 3′-((2-amino-2-oxoethoxy)methyl)-5-(2,4-bis(benzyloxy)-5-isopropylphenyl)-N-ethyl-4,5′-biisoxazole-3-carboxamide (29.5 mg, 0.047 mmol) in a yield of 89%.

¹H-NMR (400 MHz, CDCl₃) δ 7.40-7.25 (m, 9H), 7.14-7.11 (m, 2H), 6.91 (t, 1H), 6.84 (s, 1H), 6.54 (s, 1H), 6.49 (s, 1H), 5.70 (s, 1H), 5.04 (s, 2H), 4.84 (s, 2H), 4.53 (s, 2H), 3.97 (s, 2H), 3.48 (q, 2H), 3.32 (m, 1H), 1.28-1.21 (m, 9H)

### Step 2

### 3′-((2-Amino-2-oxoethoxy)methyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (30.8 mg, 0.077 mmol) in a yield of 99%.

¹H-NMR (400 MHz, CD₃OD) δ 7.16 (s, 1H), 6.60 (s, 1H), 6.37 (s, 1H), 4.67 (s, 2H), 3.99 (s, 2H), 3.44-3.39 (m, 2H), 3.19 (m, 1H), 1.23 (t, 3H), 1.18 (d, 6H)

### Example 37

### 5-(2,4-Dihydroxy-5-isopropylphenyl)-N-ethyl-3′-methyl-4,5′-biisoxazole-3-carboxamide (I-37)

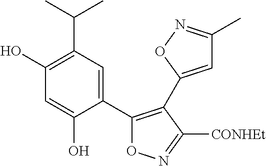

### Step 1

### 5-(2,4-Bis(benzyloxy)-5-isopropylphenyl)-N-ethyl-3′-methyl-4,5′-biisoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (102 mg, 0.17 mmol) was reacted with tetrakis(triphenylphosphine)palladium(0) (9.9 mg, 0.001 mmol) and ethyl 5-(tributylstannyl)isoxazole-3-carboxylate (76.3 mg, 0.205 mmol) to afford the intermediate compound 5-(2,4-bis(benzyloxy)-5-isopropylphenyl)-N-ethyl-3′-methyl-4,5′-biisoxazole-3-carboxamide (57.9 mg, 0.104 mmol) in a yield of 78%.

¹H-NMR (400 MHz, CDCl₃) δ 7.40-7.26 (m, 9H), 7.15-7.13 (m, 2H), 6.80 (s, 1H), 6.58 (s, 1H), 6.53 (s, 1H), 5.04 (s, 2H), 4.84 (s, 2H), 4.48 (q, 2H), 3.32 (m, 1H), 2.19 (s, 3H), 1.28-1.21 (m, 9H)

### Step 2

### 5-(2,4-Dihydroxy-5-isopropylphenyl)-N-ethyl-3′-methyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (35 mg, 0.094 mmol) in a yield of 91%.

¹H-NMR (400 MHz, CD₃OD) δ 7.11 (s, 1H), 6.37 (s, 2H), 3.41 (q, 2H), 3.18 (m, 1H), 2.26 (s, 3H), 1.25-1.16 (m, 9H)

### Example 38

### 5-(2,4-Dihydroxy-5-isopropylphenyl)-N-ethyl-3′-(piperidin-1-ylmethyl)-4,5′-biisoxazole-3-carboxamide (I-38)

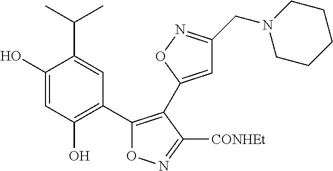

### Step 1

### 5-(2,4-Bis(benzyloxy)-5-isopropylphenyl)-N-ethyl-3′-(piperidin-1-ylmethyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (102 mg, 0.16 mmol) was reacted with piperidine (62.8 μl, 0.64 mmol) to afford the intermediate compound 5-(2,4-bis(benzyloxy)-5-isopropylphenyl)-N-ethyl-3′-(piperidin-1-ylmethyl)-4,5′-biisoxazole-3-carboxamide (96 mg, 0.15 mmol) in a yield of 95%.

¹H-NMR (400 MHz, CDCl₃) δ 7.41-7.25 (m, 9H), 7.16-7.14 (m, 2H), 6.83-6.81 (m, 2H), 6.51 (s, 1H), 5.01 (s, 2H), 4.88 (s, 2H), 3.51-3.48 (m, 4H), 3.30 (sept, 1H), 2.40-2.38 (m, 4H), 1.54-1.49 (m, 4H), 1.37-1.36 (m, 2H), 1.26 (t, 3H), 1.20 (d, 6H)

### Step 2

### 5-(2,4-Dihydroxy-5-isopropylphenyl)-N-ethyl-3′-(piperidin-1-ylmethyl)-4,5′-biisoxazole-3-carboxamide

This compound was made using the procedure described for example 1 (Step 3). Thus, this intermediate compound (Step 1) (96 mg, 0.15 mmol) was reacted with BCl₃ to afford a crude product, which was purified by silica gel column chromatography to afford the title compound (61 mg, 0.14 mmol) in a yield of 89%.

¹H-NMR (400 MHz, CD₃OD) δ 7.13 (s, 1H), 6.55 (s, 1H), 6.39 (s, 1H), 3.62 (s, 2H), 3.40 (q, 2H), 3.18 (sept, 1H), 2.53-2.49 (m, 4H), 1.64-1.58 (m, 4H), 1.47-1.46 (m, 2H), 1.23 (t, 3H), 1.18 (d, 6H)

### Example 39

### 5-(2,4-Dihydroxy-5-isopropylphenyl)-N-ethyl-3′-(pyrrolidin-1-ylmethyl)-4,5′-biisoxazole-3-carboxamide (I-39)

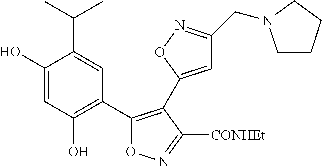

### Step 1

### 5-(2,4-Bis(benzyloxy)-5-isopropylphenyl)-N-ethyl-3′-(pyrrolidin-1-ylmethyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4, 5′-biisoxazol-3′-yl)methyl methanesulfonate (46.2 mg, 0.072 mmol) was reacted with pyrrolidine (25 μl, 0.286 mmol) to afford the intermediate compound 5-(2,4-bis(benzyloxy)-5-isopropylphenyl)-N-ethyl-3′-(pyrrolidin-1-ylmethyl)-4,5′-biisoxazole-3-carboxamide (44.4 mg, 0.072 mmol) in a yield of 99%.

¹H-NMR (400 MHz, CDCl₃) δ 7.40-7.26 (m, 9H), 7.14 (m, 2H), 6.83 (s, 1H), 6.79 (t, 1H), 6.52 (s, 1H), 5.02 (s, 2H), 4.88 (s, 2H), 3.65 (s, 2H), 3.49 (m, 2H), 3.31 (m, 1H), 2.52 (s, 4H), 1.74 (m, 4H), 1.27 (t, 3H), 1.21 (d, 6H)

### Step 2

### 5-(2,4-Dihydroxy-5-isopropylphenyl)-N-ethyl-3′-(pyrrolidin-1-ylmethyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (31.5 mg, 0.0715 mmol) in a yield of 99%.

¹H-NMR (400 MHz, CD₃OD) δ 7.14 (s, 1H), 6.55 (s, 1H), 6.37 (s, 1H), 3.74 (s, 2H), 3.41 (q, 2H), 3.18 (m, 1H), 2.64 (s, 4H), 1.83 (m, 4H), 1.23 (t, 3H), 1.18 (d, 6H)

### Example 40

### 5-(2,4-Dihydroxy-5-isopropylphenyl)-N-ethyl-3′-((isopropylamino)methyl)-4,5′-biisoxazole-3-carboxamide (I-40)

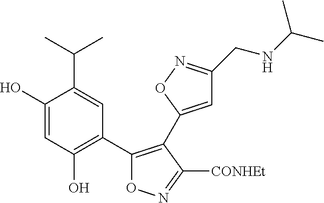

### Step 1

### 5-(2,4-Bis(benzyloxy)-5-isopropylphenyl)-N-ethyl-3′-((isopropylamino)methyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (42 mg, 0.065 mmol) was reacted with isopropylamine (22.3 μl, 0.26 mmol) to afford the intermediate compound 5-(2,4-bis(benzyloxy)-5-isopropylphenyl)-N-ethyl-3′-((isopropylamino)methyl)-4,5′-biisoxazole-3-carboxamide (35 mg, 0.057 mmol) in a yield of 88%.

¹H-NMR (400 MHz, CDCl₃) δ 7.41-7.26 (m, 9H), 7.15-7.13 (m, 2H), 6.83-6.81 (m, 1H), 6.76 (s, 1H), 6.52 (s, 1H), 5.02 (s, 2H), 4.86 (s, 2H), 3.76 (s, 2H), 3.52-3.45 (m, 2H), 3.31 (sept, 1H), 2.81 (sept, 1H), 1.26 (t, 3H), 1.21 (d, 6H), 1.04 (d, 6H)

### Step 2

### 5-(2,4-Dihydroxy-5-isopropylphenyl)-N-ethyl-3′-((isopropylamino)methyl)-4,5′-biisoxazole-3-carboxamide

This compound was made using the procedure described for example 1 (Step 3). Thus, this intermediate compound (Step 1) (35 mg, 0.057 mmol) was reacted with BCl₃ to afford a crude product, which was purified by silica gel column chromatography to afford the title compound (22 mg, 0.053 mmol) in a yield of 94%.

¹H-NMR (400 MHz, CD₃OD) δ 7.13 (s, 1H), 6.58 (s, 1H), 6.37 (s, 1H), 3.94 (s, 2H), 3.42 (q, 2H), 3.18 (sept, 1H), 2.99-2.96 (m, 1H), 1.25 (s, 3H), 1.18-1.14 (s, 12H)

### Example 41

### 5-(2,4-Dihydroxy-5-isopropylphenyl)-3′-((dimethylamino)methyl)-N-ethyl-4,5′-biisoxazole-3-carboxamide (I-41)

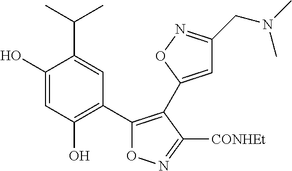

### Step 1

### 5-(2,4-Bis(benzyloxy)-5-isopropylphenyl)-3′-((dimethylamino)methyl)-N-ethyl-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4, 5′-biisoxazol-3′-yl)methyl methanesulfonate (55.1 mg, 0.085 mmol) was reacted with aqueous 50% dimethylamine (0.5 ml, 0.34 mmol) to afford the intermediate compound 5-(2,4-bis(benzyloxy)-5-isopropylphenyl)-3′-((dimethylamino)methyl)-N-ethyl-4,5′-biisoxazole-3-carboxamide (50.8 mg, 0.085 mmol) in a yield of 99%.

¹H-NMR (400 MHz, CDCl₃) δ 7.41-7.25 (m, 9H), 7.16 (m, 2H), 6.83 (t, 2H), 6.52 (s, 1H), 5.02 (s, 2H), 4.88 (s, 2H), 3.52-3.45 (m, 4H), 3.30 (m, 1H), 2.23 (s, 6H), 1.26 (t, 3H), 1.20 (d, 6H)

### Step 2 CL 5-(2,4-Dihydroxy-5-isopropylphenyl)-3′-((dimethylamino)methyl)-N-ethyl-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (30.3 mg, 0.0731 mmol) in a yield of 86%.

¹H-NMR (400 MHz, CD₃OD) δ 7.16 (s, 1H), 6.57 (s, 1H), 6.38 (s, 1H), 3.62 (s, 2H), 3.43 (q, 2H), 3.19 (m, 1H), 2.34 (s, 6H), 1.25 (t, 3H), 1.18 (d, 6H)

### Example 42

### 5-(2,4-Dihydroxy-5-isopropylphenyl)-N-ethyl-3′-((methylamino)methyl)-4,5′-bii soxazole-3-carboxamide (I-42)

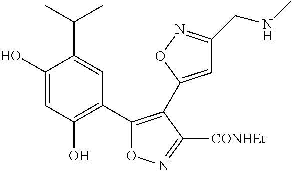

### Step 1

### 5-(2,4-Bis(benzyloxy)-5-isopropylphenyl)-N-ethyl-3′-((methylamino)methyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (50.9 mg, 0.08 mmol) was reacted with 40% aqueous methylamine (0.5 ml, 0.32 mmol) to afford the intermediate compound 5-(2,4-bis(benzyloxy)-5-isopropylphenyl)-N-ethyl-3′-((methylamino)methyl)-4,5′-biisoxazole-3-carboxamide (45.8 mg, 0.08 mmol) in a yield of 99%.

¹H-NMR (400 MHz, CDCl₃) δ 7.40-7.26 (m, 9H), 7.15 (m, 2H), 6.84 (s, 1H), 6.77 (d, 1H), 6.53 (s, 1H), 5.03 (s, 2H), 4.86 (s, 2H), 3.72 (s, 2H), 3.48 (m, 2H), 3.32 (m, 1H), 2.40 (s, 3H), 1.25 (t, 3H), 1.20 (d, 6H)

### Step 2

### 5-(2,4-Dihydroxy-5-isopropylphenyl)-N-ethyl-3′-((methylamino)methyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (29.0 mg, 0.07 mmol) in a yield of 92%.

¹H-NMR (400 MHz, CD₃OD) δ 7.18 (s, 1H), 6.62 (s, 1H), 6.38 (s, 1H), 4.01 (s, 2H), 3.43 (q, 2H), 3.19 (m, 1H), 2.57 (s, 3H), 1.23 (t, 3H), 1.19 (d, 6H)

### Example 43

### 5-(2,4-Dihydroxy-5-isopropylphenyl)-N-ethyl-3′-((4-methylpiperazin-1-yl)methyl)-4,5′-biisoxazole-3-carboxamide (I-43)

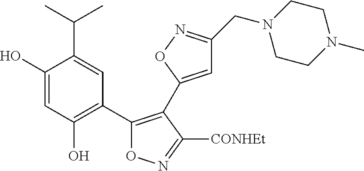

### Step 1

### 5-(2,4-Bis(benzyloxy)-5-isopropylphenyl)-N-ethyl-3′-((4-methylpiperazin-1-yl)methyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (49.8 mg, 0.08 mmol) was reacted with 1-methylpiperazine (34.2 μl, 0.31 mmol) to afford the intermediate compound 5-(2,4-bis(benzyloxy)-5-isopropylphenyl)-N-ethyl-3′-((4-methylpiperazin-1-yl)methyl)-4,5′-biisoxazole-3-carboxamide (50.1 mg, 0.08 mmol) in a yield of 99%.

¹H-NMR (400 MHz, CDCl₃) δ 7.41-7.25 (m, 9H), 7.15 (m, 2H), 6.85 (s, 1H), 6.52 (s, 1H), 5.02 (s, 2H), 4.88 (s, 2H), 3.51 (s, 2H), 3.47 (m, 2H), 3.31 (m, 1H), 2.48 (br, 6H), 2.26 (s, 3H), 1.28-1.25 (m, 3H), 1.21 (d, 6H)

### Step 2

### 5-(2,4-Dihydroxy-5-isopropylphenyl)-N-ethyl-3′-((4-methylpiperazin-1-yl)methyl)-4,5′-biisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (29.4 mg, 0.062 mmol) in a yield of 81%.

¹H-NMR (400 MHz, CD₃OD) δ 7.17 (s, 1H), 6.48 (s, 1H), 6.38 (s, 1H), 3.64 (s, 2H), 3.41 (q, 2H), 3.19 (m, 1H), 2.53 (br, 6H), 2.31 (s, 3H), 1.28 (s, 2H), 1.22 (t, 3H), 1.19 (d, 6H)

### Example 44

### 3′-((Allylamino)methyl)-5-(2,4-dihydroxy-5-isopropylphenyl)-N-ethyl-4,5′-biis oxazole-3-carboxamide (I-44)

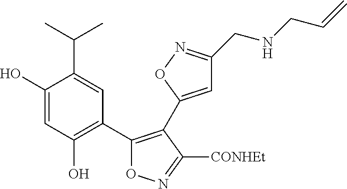

### Step 1

### 3′-((Allylamino)methyl)-5-(2,4-bis(benzyloxy-5-isopropylphenyl)-N-ethyl-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (50.5 mg, 0.078 mmol) was reacted with allylamine (23.5 μl, 0.313 mmol) to afford the intermediate compound 3′-((allylamino)methyl)-5-(2,4-bis(benzyloxy)-5-isopropylphenyl)-N-ethyl-4,5′-biisoxazole-3-carboxamide (32.6 mg, 0.054 mmol) in a yield of 69%.

¹H-NMR (400 MHz, CDCl₃) δ 7.39-7.25 (m, 9H), 7.15-7.13 (m, 2H), 6.84-6.83 (m, 1H), 6.76 (s, 1H), 6.52 (s, 1H), 5.86 (ddt, 1H), 5.19-5.11 (m, 2H), 5.02 (s, 2H), 4.86 (s, 2H), 3.76 (s, 2H), 3.51-3.44 (m, 2H), 3.33-3.28 (m, 1H), 3.25-3.23 (m, 2H), 1.25 (t, 3H), 1.21 (d, 6H)

### Step 2

3′-((Allylamino)methyl)-5-(2,4-dihydroxy-5-isopropylphenyl)-N-ethyl-4,5′-biis oxazole-3-carboxamide

This compound was made using the procedure described for example 1 (Step 3). Thus, this intermediate compound (Step 1) (32 mg, 0.078 mmol) was reacted with BCl₃ to afford a crude product, which was purified by silica gel column chromatography to afford the title compound (20 mg, 0.047 mmol) in a yield of 88%.

¹H-NMR (400 MHz, CD₃OD) δ 7.13 (s, 1H), 6.54 (s, 1H), 6.36 (s, 1H), 5.88 (ddt, 1H), 5.22 (dd, 1H), 5.16 (dd, 1H), 3.79 (s, 2H), 3.42 (q, 2H), 3.23-3.16 (m, 3H), 1.23 (t, 3H), 1.16 (d, 6H)

### Example 45

### 5-(2,4-Dihydroxy-5-isopropylphenyl)-3′-((dipropylamino)methyl)-N-ethyl-4,5′-biisoxazole-3-carboxamide (I-45)

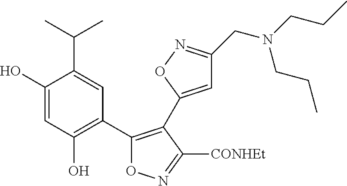

### Step 1

### 5-(2,4-Bis(benzyloxy)-5-isopropylphenyl)-3′-((dipropylamino)methyl)-N-ethyl-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (50.5 mg, 0.078 mmol) was reacted with dipropylamine (42.9 μl, 0.31 mmol) to afford the intermediate compound 5-(2,4-Bis(benzyloxy)-5-isopropylphenyl)-3′-((dipropylamino)methyl)-N-ethyl-4,5′-biisoxazole-3-carboxamide (42.8 mg, 0.066 mmol) in a yield of 84%.

¹H-NMR (400 MHz, CDCl₃) δ 7.40-7.24 (m, 9H), 7.17-7.15 (m, 2H), 6.83-6.82 (m, 1H), 6.78 (s, 1H), 6.51 (s, 1H), 5.00 (s, 2H), 4.88 (s, 2H), 3.62 (s, 2H), 3.49-3.46 (m, 2H), 3.30 (sept, 1H), 2.37-2.33 (m, 4H), 1.48-1.42 (m, 4H), 1.24 (t, 3H), 1.20 (d, 6H), 0.83 (t, 6H)

### Step 2

### 5-(2,4-Dihydroxy-5-isopropylphenyl)-3′-((dipropylamino)methyl)-N-ethyl-4,5′-biisoxazole-3-carboxamide

This compound was made using the procedure described for example 1 (Step 3). Thus, this intermediate compound (Step 1) (42 mg, 0.065 mmol) was reacted with BCl₃ to afford a crude product, which was purified by silica gel column chromatography to afford the title compound (32.3 mg, 0.068 mmol) in a yield of 99%.

¹H-NMR (400 MHz, CD₃OD) δ 7.11 (s, 1H), 6.48 (s, 1H), 6.38 (s, 1H), 3.68 (s, 2H), 3.42 (q, 2H), 3.18 (sept, 1H), 2.42-2.38 (m, 4H), 1.50 (sext, 4H), 1.23 (t, 3H), 1.18 (d, 6H), 0.88 (t, 6H)

### Example 46

### 5-(2,4-Dihydroxy-5-isopropylphenyl)-N-ethyl-3′-(thiophen-3-yl)-4,5′-biisoxazole-3-carboxamide (I-46)

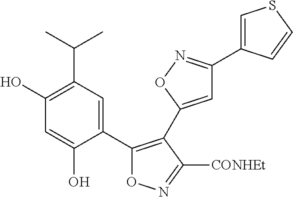

### Step 1

### 5-(2,4-Bis(benzyloxy)-5-isopropylphenyl)-N-ethyl-3′-(thiophen-3-yl)-4,5′-biisoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (123 mg, 0.21 mmol) was reacted with tetrakis(triphenylphosphine)palladium(0) (12 mg, 0.013 mmol) and 3-(thiophen-3-yl)-5-(tributylstannyl)isoxazole (182 mg, 0.41 mmol) to afford the intermediate compound 5-(2,4-bis(benzyloxy)-5-isopropylphenyl)-N-ethyl-3′-(thiophen-3-yl)-4,5′-biisoxazole-3-carboxamide (104 mg, 0.17 mmol) in a yield of 81%.

¹H-NMR (400 MHz, CDCl₃) δ 7.64 (s, 1H), 7.49-7.48 (m, 1H), 7.41-7.34 (m, 7H), 7.18-7.04 (m, 5H), 6.98 (s, 1H), 6.84-6.81 (m, 1H), 6.54 (s, 1H), 5.04 (s, 2H), 4.80 (s, 2H), 3.50-3.47 (m, 2H), 3.32 (sept, 1H), 1.28-1.22 (m, 9H)

### Step 2

### 5-(2,4-Dihydroxy-5-isopropylphenyl)-N-ethyl-3′-(thiophen-3-yl)-4,5′-biisoxazole-3-carboxamide

This compound was made using the procedure described for example 1 (Step 3). Thus, this intermediate compound (Step 1) (104 mg, 0.17 mmol) was reacted with BCl₃ to afford a crude product, which was purified by silica gel column chromatography to afford the title compound (31 mg, 0.07 mmol) in a yield of 85%.

¹H-NMR (400 MHz, CD₃OD) δ 7.90 (dd, 1H), 7.53 (dd, 1H), 7.49 (dd, 1H), 7.18 (s, 1H), 6.86 (s, 1H), 6.37 (s, 1H), 3.42 (q, 2H), 3.18 (sept, 1H), 1.24 (t, 3H), 1.18 (d, 6H)

### Example 47

### 5-(2,4-Dihydroxy-5-isopropylphenyl)-N-ethyl-3′-(thiomorpholinomethyl)-4,5′-biisoxazole-3-carboxamide (I-47)

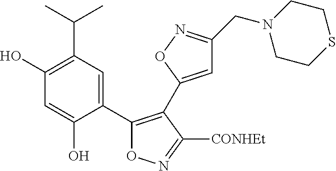

### Step 1

### 5-(2,4-Bis(benzyloxy)-5-isopropylphenyl)-N-ethyl-3′-(thiomorpholinomethyl)-4,5′-biisoxazole-3-carboxamide

This compound was made using the procedure described for example 18 (Step 2). Thus, (5-(2,4-bis(benzyloxy)-5-isopropylphenyl)-3-(ethylcarbamoyl)-4,5′-biisoxazol-3′-yl)methyl methanesulfonate (41 mg, 0.064 mmol) was reacted with thiomorpholine (22.3 μl, 0.26 mmol) to afford the intermediate compound 5-(2,4-bis(benzyloxy)-5-isopropylphenyl)-N-ethyl-3′-(thiomorpholinomethyl)-4,5′-biisoxazole-3-carboxamide (27 mg, 0.043 mmol) in a yield of 67%.

¹H-NMR (400 MHz, CDCl₃) δ 7.41-7.26 (m, 9H), 7.15-7.13 (m, 2H), 6.85-6.83 (m, 2H), 6.52 (s, 1H), 5.02 (s, 2H), 4.88 (s, 2H), 3.53 (s, 2H), 3.51-3.46 (m, 2H), 3.31 (sept, 1H), 2.69-2.66 (m, 4H), 2.61-2.58 (m, 4H), 1.27 (t, 3H), 1.21 (d, 6H)

### Step 2

### 5-(2,4-Dihydroxy-5-isopropylphenyl)-N-ethyl-3′-(thiomorpholinomethyl)-4,5′-biisoxazole-3-carboxamide

This compound was made using the procedure described for example 1 (Step 3). Thus, this intermediate compound (Step 1) (27 mg, 0.043 mmol) was reacted with BCl₃ to afford a crude product, which was purified by silica gel column chromatography to afford the title compound (21 mg, 0.045 mmol) in a yield of 99%.

¹H-NMR (400 MHz, CD₃OD) δ 7.13 (s, 1H), 6.51 (s, 1H), 6.37 (s, 1H), 3.61 (s, 2H), 3.42 (q, 2H), 3.18 (sept, 1H), 2.76-2.74 (m, 4H), 2.66-2.64 (m, 4H), 1.23 (t, 3H), 1.18 (d, 6H)

### Example 48

### 3′-Cyano-5-(2,4-dihydroxy-5-isopropylphenyl)-N-ethyl-4,5′-biisoxazole-3-carboxamide (I-48)

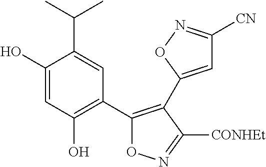

### Step 1

### 5-(2,4-Bis(benzyloxy)-5-isopropylphenyl)-3′-cyano-N-ethyl-4,5′-biisoxazole-3-carboxamide

5-(2,4-Bis(benzyloxy)-5-isopropylphenyl)-N³-ethyl-4,5′-biisoxazole-3,3′-dicarboxamide (35 mg, 0.059 mmol) was dissolved in DMF (0.7 ml), and thionyl chloride (6.5 μl, 0.089 mmol) was added. The reaciton mixture was stirred at RT for 1 h. solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-3′-cyano-N-ethyl-4,5′-biisoxazole-3-carboxamide (30 mg, 0.053 mmol) in a yield of 90%.

¹H-NMR (400 MHz, CDCl₃) δ 7.47-7.33 (m, 9H), 7.09-7.07 (m, 2H), 6.87-6.86 (m, 2H), 6.56 (s, 1H), 5.10 (s, 2H), 4.74 (s, 2H), 3.48-3.44 (m, 2H), 3.35 (sept, 1H), 1.27-1.24 (m, 9H)

### Step 2

### 3′-Cyano-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) (48 mg, 0.083 mmol) was reacted with BCl₃ to afford a crude product, which was purified by silica gel column chromatography to afford the title compound (17 mg, 0.044 mmol) in a yield of 84%.

¹H-NMR (400 MHz, CD₃OD) δ 7.25 (s, 1H), 7.01 (s, 1H), 6.35 (s, 1H), 3.42 (q, 2H), 3.18 (sept, 1H), 1.23 (t, 3H), 1.18 (d, 6H)

### Example 49

### 5-(2,4-Dihydroxy-5-isopropylphenyl)-N-ethyl-3′-phenyl-4,5′-biisoxazole-3-carb oxamide (I-49)

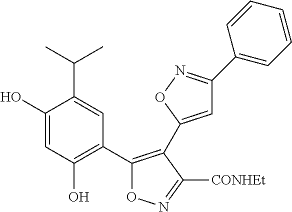

### Step 1

### 5-(2,4-Bis(benzyloxy)-5-isopropylphenyl)-N-ethyl-3′-phenyl-4,5′-biisoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (193 mg, 0.32 mmol) was reacted with tetrakis(triphenylphosphine)palladium(0) (19 mg, 0.016 mmol) and 3-phenyl-5-(tributylstannyl)isoxazole (211 mg, 0.48 mmol) to afford the intermediate compound5-(2,4-bis(benzyloxy)-5-isopropylphenyl)-N-ethyl-3′-phenyl-4,5′-biisoxazole-3-carboxamide (139 mg, 0.23 mmol) in a yield of 70%.

¹H-NMR (400 MHz, CDCl₃) δ 7.77-7.75 (m, 2H), 7.44-7.34 (m, 9H), 7.18-7.08 (m, 6H), 6.84-6.83 (m, 1H), 6.55 (s, 1H), 5.04 (s, 2H), 4.82 (s, 2H), 3.53-3.46 (m, 2H), 3.33 (sept, 1H), 1.29-1.22 (m, 9H)

### Step 2

### 5-(2,4-Dihydroxy-5-isopropylphenyl)-N-ethyl-3′-phenyl-4,5′-biisoxazole-3-carboxamide

This compound was made using the procedure described for example 1 (Step 3). Thus, this intermediate compound (Step 1) (138 mg, 0.23 mmol) was reacted with BCl₃ to afford a crude product, which was purified by silica gel column chromatography to afford the title compound (97 mg, 0.22 mmol) in a yield of 99%.

¹H-NMR (400 MHz, CD₃OD) δ 7.81-7.78 (m, 2H), 7.46-7.44 (m, 3H), 7.19 (s, 1H), 6.93 (s, 1H), 6.39 (s, 1H), 3.42 (q, 2H), 3.18 (sept, 1H), 1.24 (t, 3H), 1.17 (d, 6H)

### Example 50

### 5-(2,4-Dihydroxy-5-isopropylphenyl)-N-ethyl-3′-(thiomorpholine-4-carbonyl)-4,5′-biisoxazole-3-carboxamide (I-50)

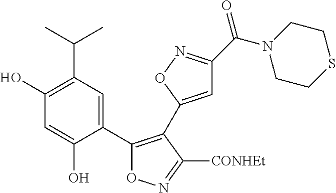

### Step 1

### 5-(2,4-Bis(benzyloxy)-5-isopropylphenyl)-N-ethyl-3′-(thiomorpholine-4-carbonyl)-4,5′-biisoxazole-3-carboxamide

This compound was made using the procedure described for example 16 (Step 1). Thus, ethyl 5-(2,4-bis(benzyloxy)-5-isopropylphenyl)-3-(ethylcarbamoyl)-4,5′-biisoxazole-3′-carboxylate (68 mg, 0.11 mmol) was reacted with thiomorpholine (104 μl, 1.12 mmol) to afford the intermediate compound 5-(2,4-bis(benzyloxy)-5-isopropylphenyl)-N-ethyl-3′-(thiomorpholine-4-carbonyl)-4,5′-biisoxazole-3-carboxamide (39 mg, 0.05 mmol) in a yield of 53%.

¹H-NMR (400 MHz, CDCl₃) δ 7.40-7.25 (m, 9H), 7.13-7.11 (m, 2H), 7.01 (s, 1H), 6.87-6.84 (m, 1H), 6.52 (s, 1H), 5.01 (s, 2H), 4.85 (s, 2H), 4.02-3.99 (m, 2H), 3.94-3.91 (m, 2H), 3.51-3.44 (m, 2H), 3.32-3.29 (m, 1H), 2.71-2.69 (m, 2H), 2.63-2.61 (m, 2H), 1.25 (t, 3H), 1.21 (d, 6H)

### Step 2

### 5-(2,4-Dihydroxy-5-isopropylphenyl)-N-ethyl-3′-(thiomorpholine-4-carbonyl)-4,5′-biisoxazole-3-carboxamide

This compound was made using the procedure described for example 1 (Step 3). Thus, this intermediate compound (Step 1) (38 mg, 0.05 mmol) was reacted with BCl₃ to afford a crude product, which was purified by silica gel column chromatography to afford the title compound (25 mg, 0.06 mmol) in a yield of 99%.

¹H-NMR (400 MHz, CD₃OD) δ 7.84 (s, 1H), 7.19 (s, 1H), 6.70 (s, 1H), 4.01-3.99 (m, 2H), 3.95-3.93 (m, 2H), 3.42 (q, 2H), 3.18 (sept, 1H), 2.73-2.67 (m, 4H), 1.22 (t, 3H), 1.18 (d, 6H)

### Example 51

### 5-(2,4-Dihydroxy-5-isopropylphenyl)-3′-(dimethoxymethyl)-N-ethyl-4,5′-biisoxa zole-3-carboxamide (I-51)

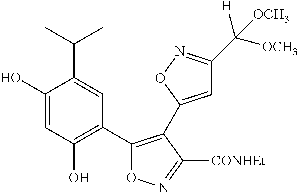

### Step 1

### 5-(2,4-Bis(benzyloxy)-5-isopropylphenyl)-N-ethyl-3′-formyl-4,5′-biisoxazole-3-carboxamide

5-(2,4-Bis(benzyloxy)-5-isopropylphenyl)-N-ethyl-3′-(hydroxymethyl)-4,5′-biisoxazole-3-carboxamide (55 mg, 0.097 mmol) was dissolved in methylene chloride (1 ml), PCC (32 mg, 0.15 mmol) was added. And the reaction mixture was stirred at RT for overnight. Solvent was evaporated in vacuo, and the residue was purified by silica gel column chromatography to afford the intermediate compound 5-(2,4-bis(benzyloxy)-5-isopropylphenyl)-N-ethyl-3′-formyl-4,5′-biisoxazole-3-carboxamide (53 mg, 0.093 mmol) in a yield of 95%.

¹H-NMR (400 MHz, CDCl₃) δ 10.02 (s, 1H), 7.42-7.32 (m, 6H), 7.26-7.24 (m, 3H), 7.07-7.05 (m, 2H), 6.99 (s, 1H), 6.89-6.86 (m, 1H), 6.54 (s, 1H), 5.05 (s, 2H), 4.79 (s, 2H), 3.50-3.43 (m, 2H), 3.33 (sept, 1H), 1.27-1.22 (m, 9H)

### Step 2

### 5-(2,4-Dihydroxy-5-isopropylphenyl)-3′-(dimethoxymethyl)-N-ethyl-4,5′-biisoxazole-3-carboxamide

This compound was made using the procedure described for example 1 (Step 3). Thus, this intermediate compound (Step 1) (52 mg, 0.093 mmol) was reacted with BCl₃ to afford a crude product, which was purified by silica gel column chromatography to afford the title compound (19 mg, 0.033 mmol) in a yield of 37%.

¹H-NMR (400 MHz, CD₃OD) δ 7.14 (s, 1H), 6.53 (s, 1H), 6.38 (s, 1H), 3.42 (q, 2H), 3.40 (s, 6H), 3.18 (sept, 1H), 1.23 (t, 3H), 1.17 (d, 6H)

### Example 52

### 5-(2,4-Dihydroxy-5-isopropylphenyl)-N-ethyl-3′-((methoxyimino)methyl)-4,5′-biisoxazole-3-carboxamide (I-52)

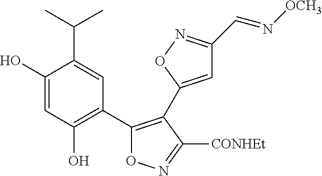

### Step 1

### 5-(2,4-Bis(benzyloxy)-5-isopropylphenyl)-N-ethyl-3′-((methoxyimino)methyl)-4,5′-biisoxazole-3-carboxamide 5-(2,4-Bis(benzyloxy)-5-isopropylphenyl)-N-ethyl-3′-formyl-4,5′-biisoxazole-3-carboxamide (50 mg, 0.09 mmol) was dissolved in methylene chloride/MeOH (0.46 ml)/(0.46 ml), methoxylamine hydrochloride (11.25 mg, 0.14 mmol) and potassium carbonate (19 mg, 0.14 mmol) were added, and the reaciton mixture was stirred at RT for overnight. solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-3′-((methoxyimino)methyl)-4,5′-biisoxazole-3-carboxamide (41 mg, 0.69 mmol) in a yield of 76%.

¹H-NMR (400 MHz, CDCl₃) δ 8.08 (s, 1H), 7.40-7.33 (m, 6H), 7.29-7.23 (m, 3H), 7.11-7.09 (m, 2H), 7.04 (s, 1H), 6.81-6.80 (m, 1H), 6.51 (s, 1H), 5.02 (s, 2H), 4.81 (s, 2H), 3.99 (s, 3H), 3.52-3.45 (m, 2H), 3.31 (sept, 1H), 1.25 (t, 3H), 1.21 (d, 6H)

### Step 2

### 5-(2,4-Dihydroxy-5-isopropylphenyl)-N-ethyl-3′-((methoxyimino)methyl)-4,5′-biisoxazole-3-carboxamide

This compound was made using the procedure described for example 1 (Step 3). Thus, this intermediate compound (Step 1) (40 mg, 0.069 mmol) was reacted with BCl₃ to afford a crude product, which was purified by silica gel column chromatography to afford the title compound (27 mg, 0.063 mmol) in a yield of 92%.

¹H-NMR (400 MHz, CD₃OD) δ 8.12 (s, 1H), 7.17 (s, 1H), 6.78 (s, 1H), 6.38 (s, 1H), 3.96 (s, 3H), 3.42 (q, 2H), 3.19 (sept, 1H), 1.23 (t, 3H), 1.18 (d, 6H)

### Example 53

### 5-(2,4-Dihydroxy-5-isopropylphenyl)-N-ethyl-3′-((hydroxyimino)methyl)-4,5′-biisoxazole-3-carboxamide (I-53)

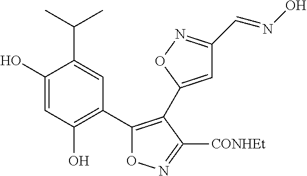

### Step 1

### 5-(2,4-Bis(benzyloxy)-5-isopropylphenyl)-N-ethyl-3′-((hydroxyimino)methyl)-4,5′-biisoxazole-3-carboxamide

This compound was made using the procedure described for example 52 (Step 1). Thus, 5-(2,4-bis(benzyloxy)-5-isopropylphenyl)-N-ethyl-3′-formyl-4,5′-biisoxazole-3-carboxamide (49 mg, 0.087 mmol) was reacted with hydroxylamine hydrochloride (8.43 mg, 0.13 mmol) to afford the intermediate compound 5-(2,4-bis(benzyloxy)-5-isopropylphenyl)-N-ethyl-3′-((hydroxyimino)methyl)-4,5′-biisoxazole-3-carboxamide (43 mg, 0.074 mmol) in a yield of 86%.

¹H-NMR (400 MHz, CDCl₃) δ 8.15 (s, 1H), 7.41-7.33 (m, 6H), 7.29-7.23 (m, 3H), 7.10-7.08 (m, 2H), 7.02 (s, 1H), 6.85-6.84 (m, 1H), 6.51 (s, 1H), 5.02 (s, 2H), 4.81 (s, 2H), 3.52-3.45 (m, 2H), 3.31 (sept, 1H), 1.25 (t, 3H), 1.21 (d, 6H)

### Step 2

5-(2,4-Dihydroxy-5-isopropylphenyl)-N-ethyl-3′-((hydroxyimino)methyl)-4,5′-bi isoxazole-3-carboxamide

This compound was made using the procedure described for example 1 (Step 3). Thus, this intermediate compound (Step 1) (43 mg, 0.074 mmol) was reacted with BCl₃ to afford a crude product, which was purified by silica gel column chromatography to afford the title compound (23 mg, 0.057 mmol) in a yield of 77%.

¹H-NMR (400 MHz, CD₃OD) δ 8.09 (s, 1H), 7.16 (s, 1H), 6.76 (s, 1H), 6.37 (s, 1H), 3.42 (q, 2H), 3.19 (sept, 1H), 1.23 (t, 3H), 1.18 (d, 6H)

### Example 54

### 3′-((Allyloxyimino)methyl)-5-(2,4-dihydroxy-5-isopropylphenyl)-N-ethyl-4,5′-biisoxazole-3-carboxamide (I-54)

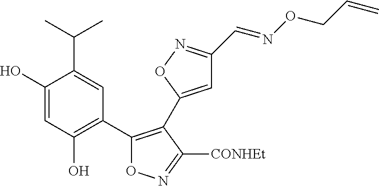

### Step 1

### 3′-((Allyloxyimino)methyl)-5-(2,4-bis(benzyloxy)-5-isopropylphenyl)-N-ethyl-4,5′-biisoxazole-3-carboxamide

This compound was made using the procedure described for example 52 (Step 1). Thus, 5-(2,4-bis(benzyloxy)-5-isopropylphenyl)-N-ethyl-3′-formyl-4,5′-biisoxazole-3-carboxamide (73 mg, 0.129 mmol) was reacted with O-allylhydroxylamine hydrochloride (21 mg, 0.19 mmol) to afford the intermediate compound 3′-((allyloxyimino)methyl)-5-(2,4-bis(benzyloxy)-5-isopropylphenyl)-N-ethyl-4,5′-biisoxazole-3-carboxamide (56 mg, 0.089 mmol) in a yield of 69%.

¹H-NMR (400 MHz, CDCl₃) δ 8.11 (s, 1H), 7.39-7.33 (m, 6H), 7.27-7.24 (m, 3H), 7.11-7.08 (m, 2H), 7.03 (s, 1H), 6.85-6.75 (m, 1H), 6.52 (s, 1H), 6.04-6.00 (m, 1H), 5.38-5.26 (m, 2H), 5.02 (s, 2H), 4.81 (s, 2H), 4.70-4.68 (m, 2H), 3.50-3.47 (m, 2H), 3.32 (sept, 1H), 1.28-1.20 (m, 9H)

### Step 2

### 3′-((Allyloxyimino)methyl)-5-(2,4-dihydroxy-5-isopropylphenyl)-N-ethyl-4,5′-biisoxazole-3-carboxamide

This compound was made using the procedure described for example 1 (Step 3). Thus, this intermediate compound (Step 1) (55 mg, 0.089 mmol) was reacted with BCl₃ to afford a crude product, which was purified by silica gel column chromatography to afford the title compound (30 mg, 0.068 mmol) in a yield of 76%.

¹H-NMR (400 MHz, CD₃OD) δ 8.17 (s, 1H), 7.17 (s, 1H), 6.78 (s, 1H), 6.38 (s, 1H), 6.00 (ddt, 1H), 5.30 (dd, 1H), 5.21 (dd, 1H), 4.67 (d, 2H), 3.42 (q, 2H), 3.19 (sept, 1H), 1.23 (t, 3H), 1.18 (d, 6H)

### Example 55

### 5-(2,4-Dihydroxy-5-isopropylphenyl)-3′-((ethoxyimino)methyl)-N-ethyl-4,5′-biisoxazole-3-carboxamide (I-55)

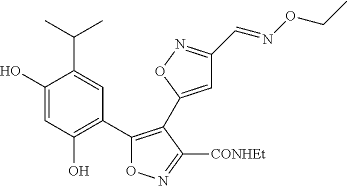

### Step 1

### 5-(2,4-Bis(benzyloxy)-5-isopropylphenyl)-3′-((ethoxyimino)methyl)-N-ethyl-4,5′-biisoxazole-3-carboxamide

This compound was made using the procedure described for example 52 (Step 1). Thus, 5-(2,4-bis(benzyloxy)-5-isopropylphenyl)-N-ethyl-3′-formyl-4,5′-biisoxazole-3-carboxamide (64 mg, 0.11 mmol) was reacted with O-ethylhydroxylamine hydrochloride (17 mg, 0.17 mmol) to afford the intermediate compound 5-(2,4-bis(benzyloxy)-5-isopropylphenyl)-3′-((ethoxyimino)methyl)-N-ethyl-4,5′-biisoxazole-3-carboxamide (49 mg, 0.08 mmol) in a yield of 72%.

¹H-NMR (400 MHz, CDCl₃) δ 8.07 (s, 1H), 7.51-7.32 (m, 6H), 7.29-7.25 (m, 3H), 7.11-7.09 (m, 2H), 7.03 (s, 1H), 6.82-6.81 (m, 1H), 6.51 (s, 1H), 5.02 (s, 2H), 4.81 (s, 2H), 4.28-4.22 (m, 2H), 3.52-3.45 (m, 2H), 3.31 (sept, 1H), 1.35-1.22 (m, 12H)

### Step 2

### 5-(2,4-Dihydroxy-5-isopropylphenyl)-3′-((ethoxyimino)methyl)-N-ethyl-4,5′-biisoxazole-3-carboxamide

This compound was made using the procedure described for example 1 (Step 3). Thus, this intermediate compound (Step 1) (49 mg, 0.08 mmol) was reacted with BCl₃ to afford a crude product, which was purified by silica gel column chromatography to afford the title compound (35 mg, 0.08 mmol) in a yield of 99%.

¹H-NMR (400 MHz, CD₃OD) δ 8.12 (s, 1H), 7.17 (s, 1H), 6.79 (s, 1H), 6.38 (s, 1H), 4.22 (q, 2H), 3.42 (q, 2H), 3.20 (sept, 1H), 1.29 (t, 3H), 1.23 (t, 3H), 1.18 (d, 6H)

### Example 56

### 5-(2,4-Dihydroxy-5-isopropylphenyl)-N³-ethyl-N^{3′}-methyl-4,5′-biisoxazole-3,3′-d icarboxamide (I-56)

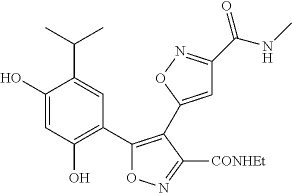

### Step 1

### 5-(2,4-Bis(benzyloxy)-5-isopropylphenyl)-N³-ethyl-N^{3′}-methyl-4,5′-biisoxazole-3,3′-dicarboxamide

This compound was made using the procedure described for example 16 (Step 1). Thus, ethyl 5-(2,4-bis(benzyloxy)-5-isopropylphenyl)-3-(ethylcarbamoyl)-4,5′-biisoxazole-3′-carboxylate (37.4 mg, 0.061 mmol) was reacted with aqueous 40% methylamine (47.6 μl, 0.061 mmol) to afford the intermediate compound 5-(2,4-bis(benzyloxy)-5-isopropylphenyl)-N³-ethyl-N^{3′}-methyl-4,5′-biisoxazole-3,3′-dicarboxamide (32.6 mg, 0.055 mmol,) in a yield of 90%.

¹H-NMR (400 MHz, CDCl₃) δ 7.41-7.25 (m, 9H), 7.09-7.06 (m, 2H), 7.00 (s, 1H), 6.86-6.80 (m, 2H), 6.50 (s, 1H), 5.01 (s, 2H), 4.81 (s, 2H), 3.49-3.42 (m, 2H), 3.31 (sept, 1H), 2.94 (d, 3H), 1.28-1.20 (m, 9H)

### Step 2

### 5-(2,4-Dihydroxy-5-isopropylphenyl)-N³-ethyl-N^{3′}-methyl-4,5′-biisoxazole-3,3′-dicarboxamide

This compound was made using the procedure described for example 1 (Step 3). Thus, this intermediate compound (Step 1) (32 mg, 0.055 mmol) was reacted with BCl₃ to afford a crude product, which was purified by silica gel column chromatography to afford the title compound (13 mg, 0.032 mmol) in a yield of 58%.

¹H-NMR (400 MHz, CD₃OD) δ 7.17 (s, 1H), 6.81 (s, 1H), 6.37 (s, 1H), 3.42 (q, 2H), 3.18 (sept, 1H), 2.89 (s, 3H), 1.23 (t, 3H), 1.18 (d, 6H)

### Example 57

### 5-(2,4-Dihydroxy-5-isopropylphenyl)-N-ethyl-4-(5-(trifluoromethyl)-1,2,4-oxadiazol-3-yl)isoxazole-3-carboxamide (I-57)

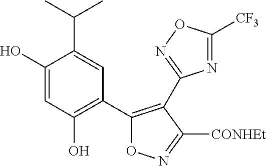

### Step 1

### 5-(2,4-Bis(benzyloxy)-5-isopropylphenyl)-4-cyano-N-ethylisoxazole-3-carboxamide

5-(2,4-Bis(benzyloxy)-5-isopropylphenyl)-N-ethyl-4-iodoisoxazole-3-carboxamide (2.18 g, 3.65 mmol) was dissolved in anhydrous CH₃CN (35 ml), tetrakis(triphenylphosphine)palladium(0) (211 mg, 0.18 mmol) and copper cyanide(I) (CuCN) (1.31 g, 14.6 mmol) were added. The reaction mixture was heated to reflux for overnight under a nitrogen atmosphere. The reaction mixture was allowed to warm to RT, and ethyl acetate was added. And then, the reaction mixture was filtered through a pad of Celite 545, and the filtrate was concentrated. The residue was purified by silica gel column chromatography to afford the intermediate compound 5-(2,4-bis(benzyloxy)-5-isopropylphenyl)-4-cyano-N-ethylisoxazole-3-carboxami de (1.16 g, 2.34 mmol) in a yield of 64%.

¹H-NMR (400 MHz, CDCl₃) δ 7.58 (s, 1H), 7.41-7.29 (m, 10H), 6.71 (t, 1H), 6.53 (s, 1H), 5.25 (s, 2H), 5.01 (s, 2H), 3.53 (m, 2H), 3.30 (sept, 1H), 1.28 (t, 3H), 1.23 (d, 6H)

### Step 2

### 5-(2,4-Bis(benzyloxy)-5-isopropylphenyl)-N-ethyl-4-(N′-hydroxycarbamimidoyl)isoxazole-3-carboxamide

The intermediate compound (Step 1) was dissolved in EtOH (15 ml), hydroxylamine hydrochloride (468 mg, 6.73 mmol) and NaHCO₃ (565 mg, 6.73 mmol) were added, and the reaction mixture was heated to reflux for 16 h. The reaction was allowed to warm to RT, and methylene chloride added. The reaction mixture was filtered, and the filtrate concentrated. The residue was purified by silica gel column chromatography to afford the intermediate compound 5-(2,4-Bis(benzyloxy)-5-isopropylphenyl)-N-ethyl-4-(N′-hydroxycarbamimidoyl)isoxazole-3-carboxamide (690 mg, 1.30 mmol) in a yield of 97%.

¹H-NMR (400 MHz, CDCl₃) δ 7.40-7.28 (m, 11H), 7.07 (br m, 1H), 6.52 (s, 1H), 5.53 (s, 2H), 5.07 (s, 2H), 5.04 (s, 1H), 4.99 (s, 2H), 3.48 (m, 2H), 3.29 (sept, 1H), 1.26 (t, 3H), 1.21 (d, 6H)

### Step 3

### 5-(2,4-Bis(benzyloxy)-5-isopropylphenyl)-N-ethyl-4-(5-(trifluoromethyl)-1,2,4-oxadiazol-3-yl)isoxazole-3-carboxamide

The intermediate compound (Step 2) (91 mg, 0.17 mmol) was dissolved in toluene (3 ml), pyridine (20.8 μl, 0.26 mmol) was added, and the reaction mixture was cooled to 0° C. And then, trifluoroacetic anhydride (35.8 μl, 0.26 mmol) was added to the solution at the same condition. After 30 min, the mixture was warmed to RT, stirred for 1 h. and heated to reflux for 1.5 h. Solvent was evaporated in vacuo, and the residue was extracted between methylene chloride and water. The organic phase was dried with magnesium sulfate, and evaporated in vacuo to give the intermediate compound 5-(2,4-bis(benzyloxy)-5-isopropylphenyl)-N-ethyl-4-(5-(trifluoromethyl)-1,2,4-oxadiazol-3-yl)isoxazole-3-carboxamide (75 mg, 0.12 mmol) in a yield of 72%.

¹H-NMR (400 MHz, CDCl₃) δ 7.49 (s, 1H), 7.41-7.28 (m, 8H), 7.09 (m, 2H), 6.80 (br t, 1H), 6.41 (s, 1H), 4.97 (s, 2H), 4.84 (s, 2H), 3.48 (m, 2H), 3.30 (sept, 1H), 1.26 (t, 3H), 1.21 (d, 6H)

### Step 4

### 5-(2,4-Dihydroxy-5-isopropylphenyl)-N-ethyl-4-(5-(trifluoromethyl)-1,2,4-oxadiazol-3-yl)isoxazole-3-carboxamide

This compound was made using the procedure described for example 1 (Step 3). Thus, this intermediate compound (Step 3) was reacted with BCl₃ to afford a crude product, which was purified by silica gel column chromatography to afford the title compound (50 mg, 0.12 mmol) in a yield of 95%.

¹H-NMR (400 MHz, CD₃OD) δ 7.37 (s, 1H), 6.30 (s, 1H), 3.39 (q, 2H), 3.20 (sept, 1H), 1.29-1.20 (m, 9H)

### Example 58

### Methyl 3-(5-(2,4-dihydroxy-5-isopropylphenyl)-3-(ethylcarbamoyl)isoxazol-4-yl)-1,2,4-oxadiazole-5-carboxylate (I-58)

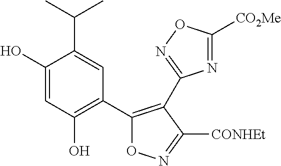

### Step 1

### Ethyl 3-(5-(2,4-bis(benzyloxy)-5-isopropylphenyl)-3-(ethylcarbamoyl)isoxazol-4-yl)-1,2,4-oxadiazole-5-carboxylate

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (300 mg, 0.57 mmol) was reacted with pyridine (0.14 ml, 1.70 mmol) and ethyl chlorooxoacetate (95.1 μl, 0.26 mmol) to afford the intermediate compound ethyl 3-(5-(2,4-bis(benzyloxy)-5-isopropylphenyl)-3-(ethylcarbamoyl)isoxazol-4-yl)-1,2,4-oxadiazole-5-carboxylate (225 mg, 0.37 mmol) in a yield of 65%.

¹H-NMR (400 MHz, CDCl₃) δ 7.52 (s, 1H), 7.39-7.28 (m, 8H), 7.08 (m, 2H), 6.86 (br t, 1H), 6.42 (s, 1H), 4.97 (s, 2H), 4.84 (s, 2H), 4.43 (q, 2H), 3.45 (m, 2H), 3.30 (sept, 1H), 1.39 (t, 3H), 1.26-1.21 (m, 9H)

### Step 2

### Methyl 3-(5-(2,4-dihydroxy-5-isopropylphenyl)-3-(ethylcarbamoyl)isoxazol-4-yl)-1,2,4-oxadiazole-5-carboxylate

This compound was made using the procedure described for example 1 (Step 3). Thus, this intermediate compound (Step 1) (20 mg, 0.03 mmol) was reacted with BCl₃ to afford a crude product, which was purified by silica gel column chromatography to afford the title compound (14 mg, 0.03 mmol) in a yield of 99%.

¹H-NMR (400 MHz, CD₃OD) δ 7.36 (s, 1H), 6.28 (s, 1H), 4.03 (s, 3H), 3.39 (q, 2H), 3.19 (sept, 1H), 1.26-1.17 (m, 9H)

### Example 59

### 3-(5-(2,4-Dihydroxy-5-isopropylphenyl)-3-(ethylcarbamoyl)isoxazol-4-yl)-N-eth yl-1,2,4-oxadiazole-5-carboxamide (I-59)

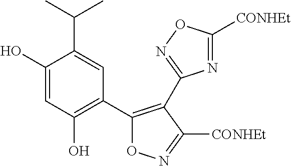

### Step 1

### 3-(5-(2,4-Bis(benzyloxy)-5-isopropylphenyl)-3-(ethylcarbamoyl)isoxazol-4-N-ethyl-1,2,4-oxadiazole-5-carboxamide

This compound was made using the procedure described for example 16 (Step 1). Thus, ethyl 3-(5-(2,4-bis(benzyloxy)-5-isopropylphenyl)-3-(ethylcarbamoyl) isoxazol-4-yl)-1,2,4-oxadiazole-5-carboxylate (30 mg, 0.05 mmol) was reacted with 30%-40% ethylamine in MeOH (1 ml) to afford the intermediate compound 3-(5-(2,4-bis(benzyloxy)-5-isopropylphenyl)-3-(ethylcarbamoyl)isoxazol-4-yl)-N-ethyl-1,2,4-oxadiazole-5-carboxamide (30 mg, 0.05 mmol) in a yield of 99%.

¹H-NMR (400 MHz, CDCl₃) δ 7.50 (s, 1H), 7.40-7.29 (m, 8H), 7.11 (m, 2H), 6.95 (t, 1H), 6.84 (t, 1H), 6.44 (s, 1H), 5.00 (s, 2H), 4.81 (s, 2H), 3.50-3.29 (m, 5H), 1.27-1.19 (m, 12H)

### Step 2

### 3-(5-(2,4-Dihydroxy-5-isopropylphenyl)-3-(ethylcarbamoyl)isoxazol-4-yl)-N-eth yl-1,2,4-oxadiazole-5-carboxamide

This compound was made using the procedure described for example 1 (Step 3). Thus, this intermediate compound (Step 1) (28 mg, 0.05 mmol) was reacted with BCl₃ to afford a crude product, which was purified by silica gel column chromatography to afford the title compound (16 mg, 0.04 mol) in a yield of 81%.

¹H-NMR (400 MHz, CD₃OD) δ 7.32 (s, 1H), 6.29 (s, 1H), 3.40 (quint, 4H), 3.19 (sept, 1H), 1.29-1.19 (m, 12H)

### Example 60

### 5-(2,4-Dihydroxy-5-isopropylphenyl)-N-ethyl-4-(5-meth 1-1,2,4-oxadiazol-3-isoxazole-3-carboxamide (I-60)

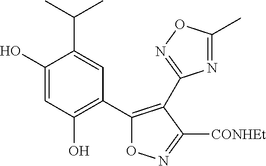

### Step 1

### 5-(2,4-Bis(benzyloxy)-5-isopropylphenyl)-N-ethyl-4-(5-methyl-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (345 mg, 0.65 mmol) was reacted with acetic anhydride (92.5 μl, 0.98 mmol) to afford the intermediate compound 5-(2,4-bis(benzyloxy)-5-isopropylphenyl)-N-ethyl-4-(5-methyl-1,2,4-oxadiazol-3-yl)isoxazole-3-carboxamide (280 mg, 0.51 mmol) in a yield of 78%.

¹H-NMR (400 MHz, CDCl₃) δ 7.46 (s, 1H), 7.40-7.27 (m, 8H), 7.16 (m, 2H), 7.00 (br t, 1H), 6.43 (s, 1H), 4.97 (s, 2H), 4.89 (s, 2H), 3.47 (m, 2H), 3.29 (sept, 1H), 2.43 (s, 3H), 1.25 (t, 3H), 1.20 (d, 6H)

### Step 2

### 5-(2,4-Dihydroxy-5-isopropylphenyl)-N-ethyl-4-(5-methyl-1,2,4-oxadiazol-3-yl) isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (140 mg, 0.38 mmol) in a yield of 74%.

¹H-NMR (400 MHz, CD₃OD) δ 7.29 (s, 1H), 6.31 (s, 1H), 3.39 (q, 2H), 3.18 (sept, 1H), 2.59 (s, 3H), 1.22 (t, 3H), 1.18 (d, 6H)

### Example 61

### 5-(2,4-Dihydroxy-5-isopropylphenyl)-N-ethyl-4-(5-phenyl-1,2,4-oxadiazol-3-yl) isoxazole-3-carboxamide (I-61)

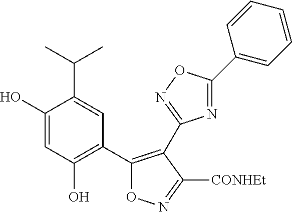

### Step 1

### 5-(2,4-Bis(benzyloxy)-5-isopropylphenyl)-N-ethyl-4-(5-phenyl-1,2,4-oxadiazol-3-yl)isoxazole-3-carboxamide

This compound was made using the procedure described for example 57 (Step 3). Thus, 5-(2,4-Bis(benzyloxy)-5-isopropylphenyl)-N-ethyl-4-(N′-hydroxycarbamimidoyl)isoxazole-3-carboxamide (60 mg, 0.11 mmol) was reacted with benzoyl chloride (19.8 μl, 0.17 mmol) to afford the intermediate compound 5-(2,4-bis(benzyloxy)-5-isopropylphenyl)-N-ethyl-4-(5-phenyl-1,2,4-oxadiazol-3-yl)isoxazole-3-carboxamide (40 mg, 0.06 mmol) in a yield of 57%.

¹H-NMR (400 MHz, CDCl₃) δ 8.04 (m, 2H), 7.59-7.46 (m, 4H), 7.38-7.30 (m, 5H), 7.18-7.16 (m, 3H), 7.10-7.08 (m, 3H), 6.43 (s, 1H), 4.96 (s, 2H), 4.85 (s, 2H), 3.48 (m, 2H), 3.28 (sept, 1H), 1.25 (t, 3H), 1.19 (d, 6H)

### Step 2

### 5-(2,4-Dihydroxy-5-isopropylphenyl)-N-ethyl-4-(5-phenyl-1,2,4-oxadiazol-3-yl) isoxazole-3-carboxamide

This compound was made using the procedure described for example 1 (Step 3). Thus, this intermediate compound (Step 1) (37 mg, 0.06 mmol) was reacted with BCl₃ to afford a crude product, which was purified by silica gel column chromatography to afford the title compound (18 mg, 0.04 mmol) in a yield of 70%.

¹H-NMR (400 MHz, CD₃OD) δ 8.14 (m, 2H), 7.66 (m, 1H), 7.58 (m, 2H), 7.34 (s, 1H), 6.31 (s, 1H), 3.40 (q, 2H), 3.18 (sept, 1H), 1.23 (t, 3H), 1.18 (d, 6H)

### Example 62

### 5-(2,4-Dihydroxy-5-isopropylphenyl)-N-ethyl-4-(5-ethyl-1,2,4-oxadiazol-3-yl)isoxazole-3-carboxamide (I-62)

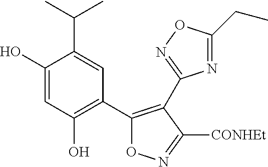

### Step 1

### 5-(2,4-Bis(benzyloxy)-5-isopropylphenyl)-N-ethyl-4-(5-ethyl-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (60 mg, 0.11 mmol) was reacted with propionyl chloride (19.7 μl, 0.23 mmol) to afford the intermediate compound 5-(2,4-bis(benzyloxy)-5-isopropylphenyl)-N-ethyl-4-(5-ethyl-1,2,4-oxadiazol-3-yl)isoxazole-3-carboxamide (58 mg, 0.10 mmol) in a yield of 67%.

¹H-NMR (400 MHz, CDCl₃) δ 7.45 (s, 1H), 7.40-7.28 (m, 8H), 7.16 (m, 2H), 7.08 (br t, 1H), 6.41 (s, 1H), 4.96 (s, 2H), 4.90 (s, 2H), 3.47 (m, 2H), 3.28 (sept, 1H), 2.79 (q, 2H), 1.30-1.21 (m, 6H), 1.19 (d, 6H)

### Step 2

### 5-(2,4-Dihydroxy-5-isopropylphenyl)-N-ethyl-4-(5-ethyl-1,2,4-oxadiazol-3-yl)isoxazole-3-carboxamide

This compound was made using the procedure described for example 1 (Step 3). Thus, this intermediate compound (Step 1) (53 mg, 0.09 mmol) was reacted with BCl₃ to afford a crude product, which was purified by silica gel column chromatography to afford the title compound (26 mg, 0.07 mmol) in a yield of 73%.

¹H-NMR (400 MHz, CD₃OD) δ 7.28 (s, 1H), 6.32 (s, 1H), 3.39 (q, 2H), 3.18 (sept, 1H), 2.95 (q, 2H), 1.38 (t, 3H), 1.22 (t, 3H), 1.18 (d, 6H)

### Example 63

### 5-(2,4-Dihydroxy-5-isopropylphenyl)-N-ethyl-4-(5-(furan-2-yl)-1,2,4-oxadiazol-3-yl)isoxazole-3-carboxamide (I-63)

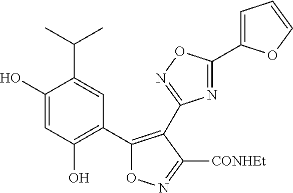

### Step 1

### 5-(2,4-Bis(benzyloxy)-5-isopropylphenyl)-N-ethyl-4-(5-(furan-2-yl)-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (70 mg, 0.13 mmol) was reacted with furan-2-carbonyl chloride (19.6 μl, 0.20 mmol) to afford the intermediate compound 5-(2,4-bis(benzyloxy)-5-isopropylphenyl)-N-ethyl-4-(5-(furan-2-yl)-1,2,4-oxadiazol-3-yl)isoxazole-3-carboxamide (43 mg, 0.07 mmol) in a yield of 53%.

¹H-NMR (400 MHz, CDCl₃) δ 7.64 (m, 1H), 7.52 (s, 1H), 7.40-7.30 (m, 6H), 7.21 (m, 3H), 7.11 (m, 2H), 7.06 (br t, 1H), 6.59 (dd, 1H), 6.43 (s, 1H), 4.96 (s, 2H), 4.86 (s, 2H), 3.47 (m, 2H), 3.29 (sept, 1H), 1.27 (t, 3H), 1.20 (d, 6H)

### Step 2

### 5-(2,4-Dihydroxy-5-isopropylphenyl)-N-ethyl-4-(5-(furan-2-yl)-1,2,4-oxadiazol-3-yl)isoxazole-3-carboxamide

This compound was made using the procedure described for example 1 (Step 3). Thus, this intermediate compound (Step 1) (39 mg, 0.06 mmol) was reacted with BCl₃ to afford a crude product, which was purified by silica gel column chromatography to afford the title compound (22 mg, 0.05 mmol) in a yield of 81%.

¹H-NMR (400 MHz, CD₃OD) δ 7.89 (dd, 1H), 7.44 (dd, 1H), 7.34 (s, 1H), 6.74 (dd, 1H), 6.30 (s, 1H), 3.40 (q, 2H), 3.18 (sept, 1H), 1.23 (t, 3H), 1.18 (d, 6H)

### Example 64

### 3-(5-(2,4-Dihydroxy-5-isopropylphenyl)-3-(ethylcarbamoyl)isoxazol-4-yl)-N,N-dimethyl-1,2,4-oxadiazole-5-carboxamide (I-64)

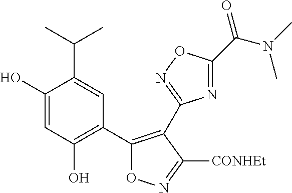

### Step 1

### 3-(5-(2,4-Bis(benzyloxy)-5-isopropylphenyl)-3-(ethylcarbamoyl)isoxazol-4-yl)-N,N-dimethyl-1,2,4-oxadiazole-5-carboxamide

This compound was made using the procedure described for example 16 (Step 1). Thus, ethyl 3-(5-(2,4-bis(benzyloxy)-5-isopropylphenyl)-3-(ethylcarbamoyl) isoxazol-4-yl)-1,2,4-oxadiazole-5-carboxylate (75 mg, 0.11 mmol) was reacted with aqueous 50% dimethylamine (0.50) to afford the intermediate compound 3-(5-(2,4-bis(benzyloxy)-5-isopropylphenyl)-3-(ethylcarbamoyl)isoxazol-4-yl)-N,N-dimethyl-1,2,4-oxadiazole-5-carboxamide (25 mg, 0.04 mmol) in a yield of 38%.

¹H-NMR (400 MHz, CDCl₃) δ 7.49 (s, 1H), 7.41-7.23 (m, 8H), 7.14 (m, 2H), 6.84 (br t, 1H), 6.43 (s, 1H), 4.97 (s, 2H), 4.84 (s, 2H), 3.46 (m, 2H), 3.29 (sept, 1H), 3.16 (s, 3H), 3.11 (s, 3H), 1.25 (t, 3H), 1.21 (d, 6H)

### Step 2

### 3-(5-(2,4-Dihydroxy-5-isopropylphenyl)-3-(ethylcarbamoyl)isoxazol-4-yl)-N,N-dimethyl-1,2,4-oxadiazole-5-carboxamide

This compound was made using the procedure described for example 1 (Step 3). Thus, this intermediate compound (Step 1) was reacted with BCl₃ to afford a crude product, which was purified by silica gel column chromatography to afford the title compound (15 mg, 0.04 mmol) in a yield of 87%.

¹H-NMR (400 MHz, CD₃OD) δ 7.34 (s, 1H), 6.30 (s, 1H), 3.39 (q, 2H), 3.24 (s, 3H), 3.19 (sept, 1H), 3.14 (s, 3H), 1.22 (t, 3H), 1.21 (d, 6H)

### Example 65

### 5-(2,4-Dihydroxy-5-isopropylphenyl)-N-ethyl-4-(5-isopropyl-1,2,4-oxadiazol-3-yl)isoxazole-3-carboxamide (I-65)

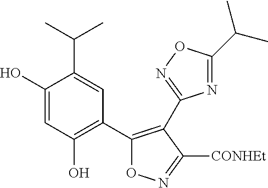

### Step 1

### 5-(2,4-Bis(benzyloxy)-5-isopropylphenyl)-N-ethyl-4-(5-isopropyl-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (70 mg, 0.13 mmol) was reacted with isobutyryl chloride (20.8 μl, 0.20 mmol) to afford the intermediate compound 5-(2,4-bis(benzyloxy)-5-isopropylphenyl)-N-ethyl-4-(5-isopropyl-1,2,4-oxadiazol-3-yl)isoxazole-3-carboxamide (60 mg, 0.10 mmol) in a yield of 78%.

¹H-NMR (400 MHz, CDCl₃) δ 7.44 (s, 1H), 7.38-7.26 (m, 8H), 7.17-7.15 (m, 3H), 6.40 (s, 1H), 4.95 (s, 2H), 4.91 (s, 2H), 3.51-3.44 (m, 2H), 3.28 (sept, 1H), 3.13 (sept, 1H), 1.32 (d, 6H), 1.25 (t, 3H), 1.19 (d, 6H)

### Step 2

### 5-(2,4-Dihydroxyl-5-isopropylphenyl)-N-ethyl-4-(5-isopropyl-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (30 mg, 0.07 mmol) in a yield of 72%.

¹H-NMR (400 MHz, CD₃OD) δ 7.27 (s, 1H), 6.32 (s, 1H), 3.38 (m, 2H), 3.29 (sept, 1H), 3.18 (sept, 1H), 1.40 (d, 6H), 1.22 (t, 3H), 1.18 (d, 6H)

### Example 66

### 5-(2,4-Dihydroxy-5-isopropylphenyl)-N-ethyl-4-(1,2,4-oxadiazol-3-yl)isoxazole-3-carboxamide (I-66)

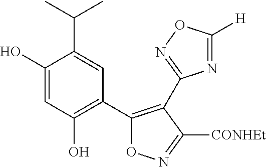

### Step 1

### 5-(2,4-Bis(benzyloxy)-5-isopropylphenyl)-N-ethyl-4-(1,2,4-oxadiazol-3-yl)isoxazole-3-carboxamide

5-(2,4-Bis(benzyloxy)-5-isopropylphenyl)-N-ethyl-4-(N′-hydroxycarbamimidoyl)isoxazole-3-carboxamide (60 mg, 0.11 mmol) was dissolved in trimethyl orthoformate (1 ml), p-toluenesulfonic acid monohydrate (2.1 mg, 0.01 mmol) was added. The reaction mixture was stirred at RT for overnight. Solvent was removed in vacuo, and the residue was dissolved in ethylacetate. And the organic phase was washed with saturated NaHCO₃, saturated aqueous NaCl, dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(1,2,4-oxadiazol-3-yl)isoxazole-3-carboxamide (45 mg, 0.08 mmol) in a yield of 73%.

¹H-NMR (400 MHz, CDCl₃) δ 8.52 (s, 1H), 7.48 (s, 1H), 7.40-7.26 (m, 8H), 7.13 (dd, 2H), 6.89 (br t, 1H), 6.44 (s, 1H), 4.98 (s, 2H), 4.84 (s, 2H), 3.48 (m, 2H), 3.30 (sept, 1H), 1.25 (t, 3H), 1.21 (d, 6H)

### Step 2

### 5-(2,4-Dihydroxy-5-isopropylphenyl)-N-ethyl-4-(1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (25 mg, 0.07 mmol) in a yield of 83%.

¹H-NMR (400 MHz, CD₃OD) δ 9.26 (s, 1H), 7.30 (s, 1H), 6.30 (s, 1H), 3.39 (q, 2H), 3.18 (sept, 1H), 1.22 (t, 3H), 1.19 (d, 6H)

### Example 67

### 5-(2,4-Dihydroxy-5-isopropylphenyl)-N-ethyl-4-(5-(morpholine-4-carbonyl)-1,2,4-oxadiazol-3-yl)isoxazole-3-carboxamide (I-67)

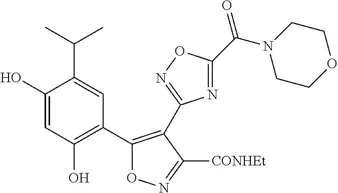

### Step 1

### 5-(2,4-Bis(benzyloxy)-5-isopropylphenyl)-N-ethyl-4-(5-(morpholine-4-carbonyl)-1,2,4-oxadiazol-3-yl)isoxazole-3-carboxamide

This compound was made using the procedure described for example 16 (Step 1). Thus, ethyl 3-(5-(2,4-bis(benzyloxy)-5-isopropylphenyl)-3-(ethylcarbamoyl) isoxazol-4-yl)-1,2,4-oxadiazole-5-carboxylate (80 mg, 0.12 mmol) was reacted with morpholine (53 μl, 0.61 mmol) to afford the intermediate compound 5-(2,4-bis(benzyloxy)-5-isopropylphenyl)-N-ethyl-4-(5-(morpholine-4-carbonyl)-1,2,4-oxadiazol-3-yl)isoxazole-3-carboxamide (60 mg, 0.09 mmol) in a yield of 75%.

¹H-NMR (400 MHz, CDCl₃) δ 7.50 (s, 1H), 7.40-7.26 (m, 8H), 7.13 (dd, 2H), 6.80 (br t, 1H), 6.43 (s, 1H), 4.97 (s, 2H), 4.83 (s, 2H), 3.76 (br t, 6H), 3.63 (br t, 2H), 3.45 (m, 2H), 3.29 (sept, 1H), 1.25 (t, 3H), 1.22 (d, 6H)

### Step 2

### 5-(2,4-Dihydroxy-5-isopropylphenyl)-N-ethyl-4-(5-(morpholine-4-carbonyl)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (36 mg, 0.08 mmol) in a yield of 83%.

¹H-NMR (400 MHz, CD₃OD) δ 7.33 (s, 1H), 6.29 (s, 1H), 3.81 (m, 2H), 3.76 (s, 4H), 3.66 (m, 2H), 3.39 (q, 2H), 3.20 (sept, 1H), 1.23 (t, 3H), 1.21 (d, 6H)

### Example 68

### 5-(2,4-Dihydroxy-5-isopropylphenyl)-N-ethyl-4-(5-(pyrrolidine-1-carbonyl)-1,2,4-oxadiazol-3-yl)isoxazole-3-carboxamide (I-68)

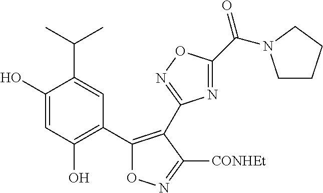

### Step 1

### 5-(2,4-Bis(benzyloxy)-5-isopropylphenyl)-N-ethyl-4-(5-(pyrrolidine-1-carbonyl)-1,2,4-oxadiazol-3-yl)isoxazole-3-carboxamide

This compound was made using the procedure described for example 16 (Step 1). Thus, ethyl 3-(5-(2,4-bis(benzyloxy)-5-isopropylphenyl)-3-(ethylcarbamoyl) isoxazol-4-yl)-1,2,4-oxadiazole-5-carboxylate (90 mg, 0.12 mmol) was reacted with pyrrolidine (114 μl, 1.37 mmol) to afford the intermediate compound 5-(2,4-bis(benzyloxy)-5-isopropylphenyl)-N-ethyl-4-(5-(pyrrolidine-1-carbonyl)-1,2,4-oxadiazol-3-yl)isoxazole-3-carboxamide (31 mg, 0.05 mmol) in a yield of 35%.

¹H-NMR (400 MHz, CDCl₃) δ 7.50 (s, 1H), 7.40-7.23 (m, 8H), 7.12 (dd, 2H), 6.87 (br t, 1H), 6.45 (s, 1H), 4.99 (s, 2H), 4.83 (s, 2H), 3.67 (br t, 2H), 3.62 (br t, 2H), 3.46 (m, 2H), 3.30 (sept, 1H), 1.23 (t, 3H), 1.18 (d, 6H)

### Step 2

### 5-(2,4-Dihydroxy-5-isopropylphenyl)-N-ethyl-4-(5-(pyrrolidine-1-carbonyl)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (17 mg, 0.04 mmol) in a yield of 76%.

¹H-NMR (400 MHz, CD₃OD) δ 7.32 (s, 1H), 6.30 (s, 1H), 3.81 (m, 2H), 3.63 (m, 2H), 3.40 (q, 2H), 3.19 (sept, 1H), 1.23 (t, 3H), 1.20 (d, 6H)

### Example 69

### 5-(2,4-Dihydroxy-5-isopropylphenyl)-N-ethyl-4-(5-(pyrrolidin-1-yl)-1,2,4-oxadiazol-3-yl)isoxazole-3-carboxamide (I-69)

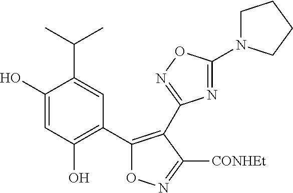

### Step 1

### 5-(2,4-Bis(benzyloxy)-5-isopropylphenyl)-N-ethyl-4-(5-(pyrrolidin-1-yl)-1,2,4-oxadiazol-3-yl)isoxazole-3-carboxamide

5-(2,4-Bis(benzyloxy)-5-isopropylphenyl)-N-ethyl-4-(5-(trichloromethyl)-1,2,4-oxadiazol-3-yl)isoxazole-3-carboxamide (148 mg, 0.23 mmol) was dissolved in DMF (3 ml), pyrrolidine (1 ml) was added. The reaciton mixture was stirred at RT for overnight. Solvent was removed in vacuo, and the residue was dissolved in ethylacetate. And the organic phase was washed with saturated 2N—HCl aqueous solution, saturated aqueous NaCl, dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(5-(pyrrolidin-1-yl)-1,2,4-oxadiazol-3-yl)isoxazole-3-carboxamide (108 mg, 0.18 mmol) in a yield of 79%.

¹H-NMR (400 MHz, CDCl₃) δ 7.77 (br t, 1H), 7.45 (s, 1H), 7.39-7.21 (m, 10H), 6.45 (s, 1H), 4.98 (s, 4H), 3.50-3.42 (m, 6H), 3.29 (sept, 1H), 1.95-1.88 (m, 4H), 1.23 (t, 3H), 1.19 (d, 6H)

### Step 2

### 5-(2,4-Dihydroxy-5-isopropylphenyl)-N-ethyl-4-(5-(pyrrolidin-1-yl)-1,2,4-oxad iazol-3-yl)isoxazole-3-carboxamide

This compound was made using the procedure described for example 1 (Step 3). Thus, this intermediate compound (Step 1) (34 mg, 0.06 mmol) was reacted with BCl₃ to afford a crude product, which was purified by silica gel column chromatography to afford the title compound (18 mg, 0.04 mmol) in a yield of 75%.

¹H-NMR (400 MHz, CD₃OD) δ 7.25 (s, 1H), 6.34 (s, 1H), 3.56 (t, 4H), 3.39 (q, 2H), 3.17 (sept, 1H), 2.04 (m, 4H), 1.22 (t, 3H), 1.15 (d, 6H)

### Example 70

### 5-(2,4-Dihydroxy-5-isopropylphenyl)-N-ethyl-4-(5-(trichloromethyl)-1,2,4-oxadiazol-3-yl)isoxazole-3-carboxamide (I-70)

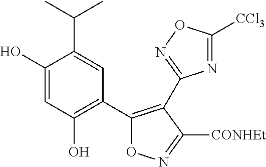

### Step 1

### 5-(2,4-Bis(benzyloxy)-5-isopropylphenyl)-N-ethyl-4-(5-(trichloromethyl)-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (100 mg, 0.19 mmol) was reacted with trichloroacetic anhydride (51.8 μl, 0.28 mmol) to afford the intermediate compound 5-(2,4-bis(benzyloxy)-5-isopropylphenyl)-N-ethyl-4-(5-(trichloromethyl)-1,2,4-oxadiazol-3-yl)isoxazole-3-carboxamide (75 mg, 0.11 mmol) in a yield of 60%.

¹H-NMR (400 MHz, CDCl₃) δ 7.48 (s, 1H), 7.38-7.26 (m, 8H), 7.13 (d, 2H), 6.83 (br, 1H), 4.94 (s, 2H), 4.90 (s, 2H), 3.48 (m, 2H), 3.29 (sept, 1H), 1.26 (t, 3H), 1.21 (d, 6H)

### Step 2

### 5-(2,4-Dihydroxy-5-isopropylphenyl)-N-ethyl-4-(5-(trichloromethyl)-1,2,4-oxadiazol-3-yl)isoxazole-3-carboxamide

This compound was made using the procedure described for example 1 (Step 3). Thus, the intermediate compound (Step 1) (35 mg, 0.23 mmol) was reacted with BCl₃ to afford a crude product, which was purified by silica gel column chromatography to afford the title compound (20 mg, 0.04 mmol) in a yield of 79%.

¹H-NMR (400 MHz, CD₃OD) δ 7.35 (s, 1H), 6.33 (s, 1H), 3.39 (q, 2H), 3.19 (sept, 1H), 1.23 (t, 3H), 1.21 (d, 6H)

### Example 71

### 5-(2,4-Dihydroxy-5-isopropylphenyl)-N-ethyl-4-(5-(piperidin-1-yl)-1,2,4-oxadiazol-3-yl)isoxazole-3-carboxamide (I-71)

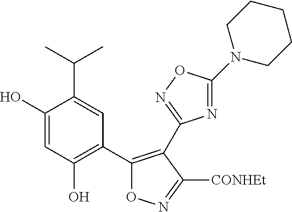

### Step 1

### 5-(2,4-Bis(benzyloxy)-5-isopropylphenyl)-N-ethyl-4-(5-(piperidin-1-yl)-1,2,4-oxadiazol-3-yl)isoxazole-3-carboxamide

This compound was made using the procedure described for example 69 (Step 1). Thus, 5-(2,4-bis(benzyloxy)-5-isopropylphenyl)-N-ethyl-4-(5-(trichloromethyl)-1,2,4-oxadiazol-3-yl)isoxazole-3-carboxamide (187.5 mg, 0.29 mmol) was reacted with piperidine (0.28 ml, 2.86 mmol) to afford the intermediate compound 5-(2,4-bis(benzyloxy)-5-isopropylphenyl)-N-ethyl-4-(5-(piperidin-1-yl)-1,2,4-oxadiazol-3-yl)isoxazole-3-carboxamide (114 mg, 0.18 mmol) in a yield of 64%.

¹H-NMR (400 MHz, CDCl₃) δ 7.69 (br t, 1H), 7.47 (s, 1H), 7.37-7.23 (m, 10H), 6.45 (s, 1H), 4.98 (s, 2H), 4.97 (s, 2H), 3.47 (br m, 6H), 3.28 (sept, 1H), 1.56 (br m, 6H), 1.23 (t, 3H), 1.19 (d, 6H)

### Step 2

### 5-(2,4-Dihydroxy-5-isopropylphenyl)-N-ethyl-4-(5-(piperidin-1-yl)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (54 mg, 0.12 mmol) in a yield of 66%.

¹H-NMR (400 MHz, CD₃OD) δ 7.24 (s, 1H), 6.35 (s, 1H), 3.58 (br m, 4H), 3.39 (q, 2H), 3.17 (sept, 1H), 1.68 (br m, 6H), 1.22 (t, 3H), 1.16 (d, 6H)

### Example 72

### 5-(2,4-Dihydroxy-5-isopropylphenyl)-4-(5-(dimethylamino)-1,2,4-oxadiazol-3-yl)-N-ethylisoxazole-3-carboxamide (I-72)

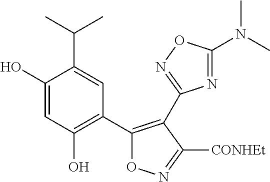

### Step 1

### 5-(2,4-Bis(benzyloxy)-5-isopropylphenyl)-4-(5-(dimethylamino)-1,2,4-oxadiazol-3-yl)-N-ethylisoxazole-3-carboxamide

This compound was made using the procedure described for example 69 (Step 1). Thus, 5-(2,4-bis(benzyloxy)-5-isopropylphenyl)-N-ethyl-4-(5-(trichloromethyl)-1,2,4-oxadiazol-3-yl)isoxazole-3-carboxamide (185 mg, 0.28 mmol) was reacted with aqueous 50% dimethylamine (1.5 ml) to afford the intermediate compound 5-(2,4-bis(benzyloxy)-5-isopropylphenyl)-4-(5-(dimethylamino)-1,2,4-oxadiazol-3-yl)-N-ethylisoxazole-3-carboxamide (135 mg, 0.23 mmol) in a yield of 82%.

¹H-NMR (400 MHz, CDCl₃) δ 7.62 (br t, 1H), 7.49 (s, 1H), 7.40-7.27 (m, 8H), 7.24 (dd, 2H), 6.46 (s, 1H), 4.98 (s, 2H), 4.97 (s, 2H), 3.47 (m, 2H), 3.29 (sept, 1H), 3.03 (s, 6H), 1.23 (t, 3H), 1.19 (d, 6H)

### Step 2

### 5-(2,4-Dihydroxy-5-isopropylphenyl)-4-(5-(dimethylamino)-1,2,4-oxadiazol-3-yl)-N-ethylisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (66 mg, 0.16 mmol) in a yield of 71%.

¹H-NMR (400 MHz, CD₃OD) δ 7.25 (s, 1H), 6.35 (s, 1H), 3.39 (q, 2H), 3.19-3.14 (m, 7H), 1.22 (t, 3H), 1.16 (d, 6H)

### Example 73

### 5-(2,4-Dihydroxy-5-isopropylphenyl)-N-ethyl-4-(5-morpholino-1,2,4-oxadiazol-3-yl)isoxazole-3-carboxamide (I-73)

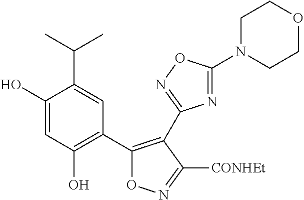

### Step 1

### 5-(2,4-Bis(benzyloxy)-5-isopropylphenyl)-N-ethyl-4-(5-morpholino-1,2,4-oxadiazol-3-yl)isoxazole-3-carboxamide

This compound was made using the procedure described for example 69 (Step 1). Thus, 5-(2,4-bis(benzyloxy)-5-isopropylphenyl)-N-ethyl-4-(5-(trichloromethyl)-1,2,4-oxadiazol-3-yl)isoxazole-3-carboxamide (148 mg, 0.23 mmol) was reacted with morpholine (1 ml) to afford the intermediate compound 5-(2,4-bis(benzyloxy)-5-isopropylphenyl)-N-ethyl-4-(5-morpholino-1,2,4-oxadiazol-3-yl)isoxazole-3-carboxamide (97 mg, 0.15 mmol) in a yield of 69%.

¹H-NMR (400 MHz, CDCl₃) δ 7.48 (s, 1H), 7.39-7.27 (m, 8H), 7.22 (dd, 2H), 6.42 (s, 1H), 4.99 (s, 2H), 4.96 (s, 2H), 3.66 (t, 4H), 3.50-3.43 (m, 6H), 3.29 (sept, 1H), 1.23 (t, 3H), 1.19 (d, 6H)

### Step 2

### 5-(2,4-Dihydroxy-5-isopropylphenyl)-N-ethyl-4-(5-morpholino-1,2,4-oxadiazol-3-yl)isoxazole-3-carboxamide

This compound was made using the procedure described for example 1 (Step 3). Thus, this intermediate compound (Step 1) (18 mg, 0.03 mmol) was reacted with BCl₃ to afford a crude product, which was purified by silica gel column chromatography to afford the title compound (9 mg, 0.02 mmol) in a yield of 70%.

¹H-NMR (400 MHz, CD₃OD) δ 7.25 (s, 1H), 6.35 (s, 1H), 3.76 (m, 4H), 3.60 (m, 4H), 3.39 (q, 2H), 3.17 (sept, 1H), 1.22 (t, 3H), 1.17 (d, 6H)

### Example 74

### 3-(5-(2,4-Dihydroxyl-5-isopropylphenyl)-3-(ethylcarbamoyl)isoxazol-4-yl)-N,N-diethyl-1,2,4-oxadiazole-5-carboxamide (I-74)

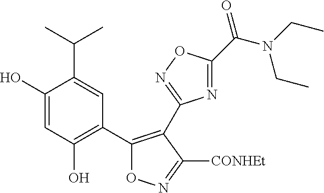

### Step 1

### 3-(5-(2,4-Bis(benzyloxy)-5-isopropylphenyl)-3-(ethylcarbamoyl)isoxazol-4-yl)-N,N-diethyl-1,2,4-oxadiazole-5-carboxamide

This compound was made using the procedure described for example 16 (Step 1) Thus, ethyl 3-(5-(2,4-bis(benzyloxy)-5-isopropylphenyl)-3-(ethylcarbamoyl) isoxazol-4-yl)-1,2,4-oxadiazole-5-carboxylate (65 mg, 0.10 mmol) was reacted with diethylamine (102.5 μl, 0.99 mmol) to afford the intermediate compound 3-(5-(2,4-bis(benzyloxy)-5-isopropylphenyl)-3-(ethylcarbamoyl)isoxazol-4-yl)-N,N-diethyl-1,2,4-oxadiazole-5-carboxamide (34 mg, 0.05 mmol) in a yield of 54%.

¹H-NMR (400 MHz, CDCl₃) δ 7.49 (s, 1H), 7.39-7.23 (m, 8H), 7.14 (dd, 2H), 6.84 (br t, 1H), 6.44 (s, 1H), 4.97 (s, 2H), 4.85 (s, 2H), 3.52 (q, 2H), 3.47-3.39 (m, 4H), 3.29 (sept, 1H), 1.27-1.20 (m, 12H), 1.16 (t, 3H)

### Step 2

### 3-(5-(2,4-Dihydroxy-5-isopropylphenyl)-3-(ethylcarbamoyl)isoxazol-4-yl)-N,N-diethyl-1,2,4-oxadiazole-5-carboxamide

This compound was made using the procedure described for example 1 (Step 3). Thus, this intermediate compound (Step 1) was reacted with BCl₃ to afford a crude product, which was purified by silica gel column chromatography to afford the title compound (21 mg, 0.04 mmol) in a yield of 86%.

¹H-NMR (400 MHz, CD₃OD) δ 7.33 (s, 1H), 6.31 (s, 1H), 3.59-3.52 (m, 4H), 3.39 (q, 2H), 3.20 (sept, 1H), 1.24 (t, 3H), 1.22 (t, 3H), 1.21 (d, 6H), 1.17 (t, 3H)

### Example 75

### 5-(2,4-Dihydroxy-5-isopropylphenyl)-N-ethyl-4-(5-(methoxymethyl)-1,2,4-oxadiazol-3-yl)isoxazole-3-carboxamide (I-75)

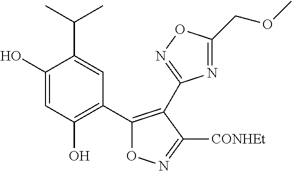

### Step 1

### 5-(2,4-Bis(benzyloxy)-5-isopropylphenyl)-N-ethyl-4-(5-(methoxymethyl)-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (60 mg, 0.11 mmol) was reacted with methoxyacetyl chloride (15.6 μl, 0.17 mmol) to afford the intermediate compound 5-(2,4-bis(benzyloxy)-5-isopropylphenyl)-N-ethyl-4-(5-(methoxymethyl)-1,2,4-oxadiazol-3-yl)isoxazole-3-carboxamide (45 mg, 0.08 mmol) in a yield of 68%.

¹H-NMR (400 MHz, CDCl₃) δ 7.47 (s, 1H), 7.39-7.26 (m, 8H), 7.15 (dd, 2H), 6.95 (br t, 1H), 6.42 (s, 1H), 4.96 (s, 2H), 4.88 (s, 2H), 4.53 (s, 2H), 3.51-3.42 (m, 5H), 3.29 (sept, 1H), 1.25 (t, 13H), 1.20 (d, 6H)

### Step 2

5-(2,4-Dihydroxy-5-isopropylphenyl)-N-ethyl-4-(5-(methoxymethyl)-1,2,4-oxadiazol-3-yl)isoxazole-3-carboxamide

This compound was made using the procedure described for example 1 (Step 3). Thus, this intermediate compound (Step 1) (40 mg, 0.07 mmol) was reacted with BCl₃ to afford a crude product, which was purified by silica gel column chromatography to afford the title compound (23 mg, 0.06 mmol) in a yield of 83%.

¹H-NMR (400 MHz, CD₃OD) δ 7.31 (s, 1H), 6.31 (s, 1H), 4.72 (s, 2H), 3.47 (s, 3H), 3.39 (q, 2H), 3.18 (sept, 1H), 1.22 (t, 3H), 1.19 (d, 6H)

### Example 76

### 4-(5-(Diethylamino)-1,2,4-oxadiazol-3-yl)-5-(2,4-dihydroxy-5-isopropylphenyl)-N-ethylisoxazole-3-carboxamide (I-76)

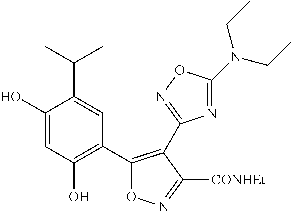

### Step 1

### 5-(2,4-Bis(benzyloxy)-5-isopropylphenyl)-4-(5-(diethylamino)-1,2,4-oxadiazol-3-yl)-N-ethylisoxazole-3-carboxamide

This compound was made using the procedure described for example 69 (Step 1). Thus, 5-(2,4-bis(benzyloxy)-5-isopropylphenyl)-N-ethyl-4-(5-(trichloromethyl)-1,2,4-oxadiazol-3-yl)isoxazole-3-carboxamide (65 mg, 0.10 mmol) was reacted with diethylamine (102.5 μl, 0.99 mmol) to afford the intermediate compound 5-(2,4-bis(benzyloxy)-5-isopropylphenyl)-4-(5-(diethylamino)-1,2,4-oxadiazol-3-yl)-N-ethylisoxazole-3-carboxamide (10 mg, 0.02 mmol) in a yield of 16%.

¹H-NMR (400 MHz, CDCl₃) δ 7.49 (s, 1H), 7.46-7.22 (m, 10H), 6.45 (s, 1H), 4.97 (s, 2H), 4.96 (s, 2H), 3.47 (m, 3H), 3.37-3.24 (m, 4H), 1.30-1.16 (m, 15H)

### Step 2

### 4-(5-(Diethylamino)-1,2,4-oxadiazol-3-yl)-5-(2,4-dihydroxy-5-isopropylphenyl)-N-ethylisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (6 mg, 0.01 mmol) in a yield of 85%.

¹H-NMR (400 MHz, CD₃OD) δ 7.24 (s, 1H), 6.36 (s, 1H), 3.42-3.33 (m, 6H), 3.17 (sept, 1H), 1.23 (t, 3H), 1.22 (t, 3H), 1.16 (d, 6H)

### Example 77

### 5-(2,4-Dihydroxy-5-isopropylphenyl)-N-ethyl-4-(5-(thiophen-2-yl)-1,2,4-oxadiazol-3-yl)isoxazole-3-carboxamide (I-77)

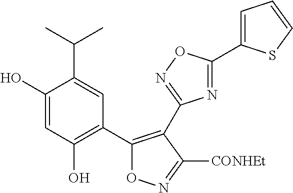

### Step 1

### 5-(2,4-Bis(benzyloxy)-5-isopropylphenyl)-N-ethyl-4-(5-(thiophen-2-yl)-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (70 mg, 0.13 mmol) was reacted with thiophene-2-carbonyl chloride (21 μl, 0.20 mmol) to afford the intermediate compound 5-(2,4-bis(benzyloxy)-5-isopropylphenyl)-N-ethyl-4-(5-(thiophen-2-yl)-1,2,4-oxadiazol-3-yl)isoxazole-3-carboxamide (35 mg, 0.06 mmol) in a yield of 43%.

¹H-NMR (400 MHz, CDCl₃) δ 7.80 (m, 1H), 7.60 (dd, 1H), 7.53 (s, 1H), 7.38-7.29 (m, 5H), 7.21-7.10 (m, 7H), 6.43 (s, 1H), 4.96 (s, 2H), 4.87 (s, 2H), 3.47 (m, 2H), 3.28 (sept, 1H), 1.24 (t, 3H), 1.20 (d, 6H)

### Step 2

### 5-(2,4-Dihydroxy-5-isopropylphenyl)-N-ethyl-4-(5-(thiophen-2-yl)-1,2,4-oxadia zol-3-yl)isoxazole-3-carboxamide

This compound was made using the procedure described for example 1 (Step 3). Thus, this intermediate compound (Step 1) (62 mg, 0.10 mmol) was reacted with BCl₃ to afford a crude product, which was purified by silica gel column chromatography to afford the title compound (20 mg, 0.05 mmol) in a yield of 80%.

¹H-NMR (400 MHz, CD₃OD) δ 7.97 (dd, 1H), 7.88 (dd, 1H), 7.33 (s, 1H), 7.28 (dd, 1H), 6.31 (s, 1H), 3.40 (q, 2H), 3.18 (sept, 1H), 1.23 (t, 3H), 1.18 (d, 6H)

### Example 78

### 4-(5-Amino-1,2,4-oxadiazol-3-yl)-5-(2,4-dihydroxy-5-isopropylphenyl)-N-ethylisoxazole-3-carboxamide (I-78)

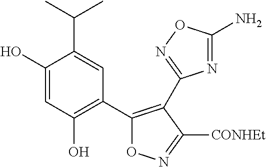

### Step 1

### 4-(5-Amino-1,2,4-oxadiazol-3-yl)-5-(2,4-bis(benzyloxy)-5-isopropylphenyl)-N-ethylisoxazole-3-carboxamide

5-(2,4-Bis(benzyloxy)-5-isopropylphenyl)-N-ethyl-4-(5-(trichloromethyl)-1,2,4-oxadiazol-3-yl)isoxazole-3-carboxamide (60 mg, 0.09 mmol) was dissolved in DMF (1 ml), ammonia water (0.5 ml) was added. The reaction mixture was stirred at RT for 2 h, and ammonia water (0.5 ml) was added. And then the reaction mixture was stirred at RT for overnight. After this time, the reaction was quenched with MeOH, solvents were removed in vacuo, and the residue was purified by silica gel column chromatography to afford the intermediate compound 4-(5-amino-1,2,4-oxadiazol-3-yl)-5-(2,4-bis(benzyloxy)-5-isopropylphenyl)-N-ethylisoxazole-3-carboxamide (41.5 mg, 0.07 mmol) in a yield of 82%.

¹H-NMR (400 MHz, CDCl₃) δ 8.06 (br t, 1H), 7.43 (s, 1H), 7.40-7.26 (m, 8H), 7.22 (m, 2H), 6.49 (s, 1H), 4.99 (s, 2H), 4.96 (s, 2H), 3.44 (m, 2H), 3.29 (sept, 1H), 1.24 (t, 3H), 1.19 (d, 6H)

### Step 2

### 4-(5-Amino-1,2,4-oxadiazol-3-yl)-5-(2,4-dihydroxy-5-isopropylphenyl)-N-ethylisoxazole-3-carboxamide

This compound was made using the procedure described for example 1 (Step 3). Thus, this intermediate compound (Step 1) (42 mg, 0.07 mmol) was reacted with BCl₃ to afford a crude product, which was purified by silica gel column chromatography to afford the title compound (24 mg, 0.06 mmol) in a yield of 87%.

¹H-NMR (400 MHz, CD₃OD) δ 7.23 (s, 1H), 6.35 (s, 1H), 3.39 (q, 2H), 3.17 (sept, 1H), 1.22 (t, 3H), 1.16 (d, 6H)

### Example 79

### 5-(2,4-Dihydroxy-5-isopropylphenyl)-N-ethyl-4-(5-(methylamino)-1,2,4-oxadiazo 1-3-yl)isoxazole-3-carboxamide (I-79)

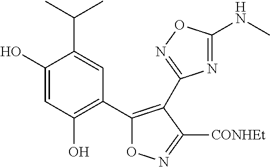

### Step 1

### 5-(2,4-Bis(benzyloxy)-5-isopropylphenyl)-N-ethyl-4-(5-(methylamino)-1,2,4-oxa diazol-3-yl)isoxazole-3-carboxamide

This compound was made using the procedure described for example 69 (Step 1). Thus, 5-(2,4-bis(benzyloxy)-5-isopropylphenyl)-N-ethyl-4-(5-(trichloromethyl)-1,2,4-oxadiazol-3-yl)isoxazole-3-carboxamide (45 mg, 0.07 mmol) was reacted with aqueous 40% methylamine (0.5 ml to afford the intermediate compound 5-(2,4-bis(benzyloxy)-5-isopropylphenyl)-N-ethyl-4-(5-(methylamino)-1,2,4-oxadiazol-3-yl)isoxazole-3-carboxamide (28.5 mg, 0.05 mmol) in a yield of 73%.

¹H-NMR (400 MHz, CDCl₃) δ 7.46 (s, 1H), 7.41-7.28 (m, 8H), 7.23 (m, 2H), 6.51 (s, 1H), 5.01 (s, 2H), 4.97 (s, 2H), 3.45 (m, 2H), 3.31 (m, 1H), 2.94 (s, 3H), 1.25 (t, 3H), 1.21 (d, 6H)

### Step 2

### 5-(2,4-Dihydroxy-5-isopropylphenyl)-N-ethyl-4-(5-(methylamino)-1,2,4-oxadiazo 1-3-yl)isoxazole-3-carboxamide

This compound was made using the procedure described for example 1 (Step 3). Thus, this intermediate compound (Step 1) (28 mg, 0.05 mmol) was reacted with BCl₃ to afford a crude product, which was purified by silica gel column chromatography to afford the title compound (16 mg, 0.04 mmol) in a yield of 84%.

¹H-NMR (400 MHz, CD₃OD) δ 7.25 (s, 1H), 6.35 (s, 1H), 3.39 (q, 2H), 3.17 (sept, 1H), 2.96 (s, 3H), 1.22 (t, 3H), 1.16 (d, 6H)

### Example 80

### 5-(2,4-Dihydroxy-5-isopropylphenyl)-N-ethyl-4-(5-hydroxy-1,2,4-oxadiazol-3-yl)isoxazole-3-carboxamide (I-80)

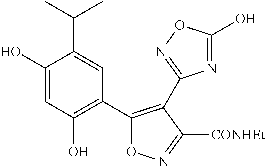

### Step 1

### 5-(2,4-Bis(benzyloxy)-5-isopropylphenyl)-N-ethyl-4-(5-hydroxy-1,2,4-oxadiazol-3-yl)isoxazole-3-carboxamide

5-(2,4-Bis(benzyloxy)-5-isopropylphenyl)-N-ethyl-4-(N′-hydroxycarbamimidoyl)isoxazole-3-carboxamide (100 mg, 0.19 mmol) was dissolved in acetone (3 ml), potassium carbonate (43.1 mg, 0.31 mmol) was added, and the reaction mixture was cooled to 0° C. Ethyl chloroformate (30 μl, 0.31 mmol) was added, and the reaction mixture was stirred at same condition for 1 h. After this time, the reaction was quenched with water, solvents were evaporated in vacuo, and the residue was extracted between methylene chloride and water. The organic phase was dried with magnesium sulfate, and evaporated in vacuo. The residue was dissolved in pyridine (3 ml), and heated to reflux for overnight. The mixture was cooled to ambient temperature, solvent was evaporated in vacuo, and the residue was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(5-hydroxy-1,2,4-oxadiazol-3-yl)isoxazole-3-carboxamide (65 mg, 0.12 mmol) in a yield of 62%.

¹H-NMR (400 MHz, CDCl₃) δ 11.4 (s, 1H), 7.42-7.28 (m, 9H), 7.24-7.18 (m, 3H), 6.63 (s, 1H), 5.10 (s, 2H), 5.00 (s, 2H), 3.51 (quint, 2H), 3.33 (sept, 1H), 1.29 (t, 3H), 1.23 (d, 6H)

### Step 2

### 5-(2,4-Dihydroxy-5-isopropylphenyl)-N-ethyl-4-(5-hydroxy-1,2,4-oxadiazol-3-yl)isoxazole-3-carboxamide

This compound was made using the procedure described for example 1 (Step 3). Thus, this intermediate compound (Step 1) (35 mg, 0.06 mmol) was reacted with BCl₃ to afford a crude product, which was purified by silica gel column chromatography to afford the title compound (22 mg, 0.06 mmol) in a yield of 93%.

¹H-NMR (400 MHz, CD₃OD) δ 7.36 (s, 1H), 6.37 (s, 1H), 3.40 (q, 2H), 3.19 (sept, 1H), 1.22 (t, 3H), 1.21 (d, 6H)

### Example 81

### 5-(2,4-Dihydroxy-5-isopropylphenyl)-N-ethyl-4-(5-(hydroxymethyl)-1,2,4-oxadiazol-3-yl)isoxazole-3-carboxamide (I-81)

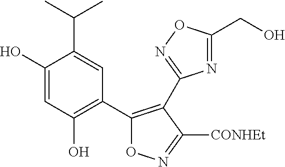

### Step 1

(3-(5-(2,4-Bis(benzyloxy)-5-isopropylphenyl)-3-(ethylcarbamoyl)isoxazol-4-yl)-1,2,4-oxadiazol-5-yl)methyl acetate

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (200 mg, 0.38 mmol) was reacted with acetoxyacetyl chloride (44.7 μl, 0.42 mmol) to afford the intermediate compound (Step 1) (3-(5-(2,4-bis(benzyloxy)-5-isopropylphenyl)-3-(ethylcarbamoyl)isoxazol-4-yl)-1,2,4-oxadiazol-5-yl)methyl acetate (167 mg, 0.27 mmol) in a yield of 72%.

¹H-NMR (400 MHz, CDCl₃) δ 7.48 (s, 1H), 7.40-7.28 (m, 8H), 7.14 (dd, 2H), 6.91 (br t, 1H), 6.40 (s, 1H), 5.15 (s, 2H), 4.95 (s, 2H), 4.87 (s, 2H), 3.47 (m, 2H), 3.29 (sept, 1H), 2.06 (s, 3H), 1.25 (t, 3H), 1.20 (d, 6H)

Step 2

### 5-(2,4-Bis(benzyloxy-5-isopropylphenyl)-N-ethyl-4-(5-(hydroxymethyl)-1,2,4-oxadiazol-3-yl)isoxazole-3-carboxamide

The intermediate compound (Step 1) was dissolved in MeOH (3 ml), potassium carbonate (41.6 mg, 0.30 mmol) was added. The reaction mixture was stirred at RT for 30 min, and removed in vacuo. The residue was purified by silica gel column chromatography to afford the intermediate compound 5-(2,4-bis(benzyloxy)-5-isopropylphenyl)-N-ethyl-4-(5-(hydroxymethyl)-1,2,4-o xadiazol-3-yl)isoxazole-3-carboxamide (142 mg, 0.25 mmol) in a yield of 91%.

¹H-NMR (400 MHz, CDCl₃) δ 7.49 (s, 1H), 7.38-7.25 (m, 8H), 7.12 (dd, 2H), 6.98 (br t, 1H), 6.42 (s, 1H), 4.96 (s, 2H), 4.86 (s, 2H), 4.62 (s, 2H), 3.43 (m, 2H), 3.29 (sept, 1H), 1.22 (t, 3H), 1.20 (d, 6H)

### Step 3

### 5-(2,4-Dihydroxy-5-isopropylphenyl)-N-ethyl-4-(5-(hydroxymethyl)-1,2,4-oxadiazol-3-yl)isoxazole-3-carboxamide

This compound was made using the procedure described for example 1 (Step 3). Thus, this intermediate compound (Step 2) (25 mg, 0.04 mmol) was reacted with BCl₃ to afford a crude product, which was purified by silica gel column chromatography to afford the title compound (16 mg, 0.04 mmol) in a yield of 94%.

¹H-NMR (400 MHz, CD₃OD) δ 7.29 (s, 1H), 6.31 (s, 1H), 4.80 (s, 2H), 3.39 (q, 2H), 3.18 (sept, 1H), 1.22 (t, 3H), 1.19 (d, 6H)

### Example 82

### 5-(2,4-Dihydroxy-5-isopropylphenyl)-N-ethyl-4-(5-mercapto-1,2,4-oxadiazol-3-yl)isoxazole-3-carboxamide (I-82)

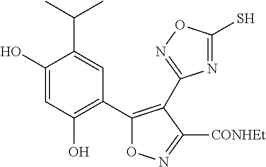

### Step 1

### 5-(2,4-Bis(benzyloxy)-5-isopropylphenyl)-N-ethyl-4-(5-mercapto-1,2,4-oxadiazol-3-yl)isoxazole-3-carboxamide

5-(2,4-Bis(benzyloxy)-5-isopropylphenyl)-N-ethyl-4-(N′-hydroxycarbamimidoyl)isoxazole-3-carboxamide (80 mg, 0.15 mmol) was suspended in CH₃CN (3 ml), 1,1-thiocarbonyldiimidazole (40.5 mg, 0.23 mmol) was added. 1,8-diazabicyclo[5,4,0]unde-7-cene (90.5 μl, 0.61 mmol) was added to this suspension, and this mixture was stirred to RT for overnight. After this time 1,1-thiocarbonyldiimidazole (67.4 mg, 0.38 mmol) was added, the mixture was stirred to RT for 3 h, and quenched with H₂O (10 ml). And then 2N—HCl was added to acidify (pH 7) the reaction mixture. And this mixture was extracted between methylene chloride and water. The organic phase was dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(5-mercapto-1,2,4-oxadiazo 1-3-yl)isoxazole-3-carboxamide (60 mg, 0.11 mmol) in a yield of 69%.

¹H-NMR (400 MHz, CDCl₃) δ 7.39-7.29 (m, 9H), 7.19 (dd, 2H), 6.58 (s, 1H), 5.07 (s, 2H), 4.96 (s, 2H), 4.62 (s, 2H), 3.47 (quint, 2H), 3.31 (sept, 1H), 1.25 (t, 3H), 1.22 (d, 6H)

### Step 2

### 5-(2,4-Dihydroxy-5-isopropylphenyl)-N-ethyl-4-(5-mercapto-1,2,4-oxadiazol-3-yl)isoxazole-3-carboxamide

This compound was made using the procedure described for example 1 (Step 3). Thus, this intermediate compound (Step 1) (45 mg, 0.08 mmol) was reacted with BCl₃ to afford a crude product, which was purified by silica gel column chromatography to afford the title compound (21 mg, 0.05 mmol) in a yield of 68%.

¹H-NMR (400 MHz, CD₃OD) δ 7.23 (s, 1H), 6.35 (s, 1H), 3.40 (q, 2H), 3.16 (sept, 1H), 1.23 (t, 3H), 1.17 (d, 6H)

### Example 83

### (S)-5-(2,4-Dihydroxy-5-isopropylphenyl)-N-ethyl-4-(5-(1-hydroxyethyl)-1,2,4-oxadiazol-3-yl)isoxazole-3-carboxamide (I-83)

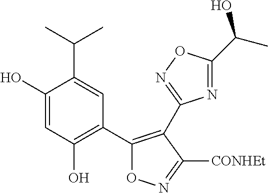

### Step 1

### (S)-1-(3-(5-(2,4-Bis(benzyloxy)-5-isopropylphenyl)-3-(ethylcarbamoyl)isoxazol-4-yl)-1,2,4-oxadiazol-5-yl)ethyl acetate

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (100 mg, 0.19 mmol) was reacted with (S)-(−)-2-acetoxypropionyl chloride (26.3 μl, 0.21 mmol) to afford the intermediate compound (S)-1-(3-(5-(2,4-bis(benzyloxy)-5-isopropylphenyl)-3-(ethylcarbamoyl)isoxazol-4-yl)-1,2,4-oxadiazol-5-yl)ethyl acetate (69 mg, 0.11 mmol) in a yield of 58%.

¹H-NMR (400 MHz, CDCl₃) δ 7.38 (s, 1H), 7.36-7.25 (m, 8H), 7.14 (dd, 2H), 7.00 (br t, 1H), 6.40 (s, 1H), 5.98 (q, 1H), 4.94 (s, 2H), 4.88 (s, 2H), 3.46 (m, 2H), 3.28 (sept, 1H), 2.02 (s, 3H), 1.61 (d, 3H), 1.24 (t, 3H), 1.20 (d, 6H)

### Step 2

### (S)-5-(2,4-Bis(benzyloxy-5-isopropylphenyl)-N-ethyl-4-(5-(1-hydroxyethyl)-1 2,4-oxadiazol-3-yl)isoxazole-3-carboxamide

This compound was made using the procedure described for example 81 (Step 2). Thus, this intermediate compound (Step 1) (45 mg, 0.07 mmol) was reacted with potassium carbonate (11 mg, 0.08 mmol) to afford the intermediate compound (S)-5-(2,4-bis(benzyloxy)-5-isopropylphenyl)-N-ethyl-4-(5-(1-hydroxyethyl)-1,2,4-oxadiazol-3-yl)isoxazole-3-carboxamide (14 mg, 0.02 mmol) in a yield of 33%.

¹H-NMR (400 MHz, CDCl₃) δ 7.47 (s, 1H), 7.38-7.27 (m, 8H), 7.13 (dd, 2H), 6.98 (br t, 1H), 6.40 (s, 1H), 4.94 (s, 2H), 4.88 (s, 2H), 3.44 (m, 2H), 3.28 (sept, 1H), 3.13 (br d, 1H), 1.50 (d, 3H), 1.23 (t, 3H), 1.20 (d, 6H)

### Step 3

### (S)-5-(2,4-Dihydroxy-5-isopropylphenyl)-N-ethyl-4-(5-(1-hydroxyethyl)-1,2,4-oxadiazol-3-yl)isoxazole-3-carboxamide

This compound was made using the procedure described for example 1 (Step 3). Thus, this intermediate compound (Step 2) (20 mg, 0.03 mmol) was reacted with BCl₃ to afford a crude product, which was purified by silica gel column chromatography to afford the title compound (13 mg, 0.03 mmol) in a yield of 94%.

¹H-NMR (400 MHz, CD₃OD) δ 7.28 (s, 1H), 6.32 (s, 1H), 5.04 (q, 1H), 3.39 (q, 2H), 3.18 (sept, 1H), 1.59 (d, 3H), 1.22 (t, 3H), 1.18 (d, 6H)

### Example 84

### 5-(2,4-Dihydroxy-5-isopropylphenyl)-N-ethyl-4-(5-(4-methoxyphenyl)-1,2,4-oxadiazol-3-yl)isoxazole-3-carboxamide (I-84)

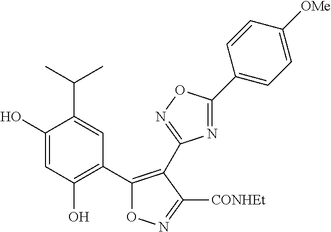

### Step 1

### 5-(2,4-Bis(benzyloxy)-5-isopropylphenyl)-N-ethyl-4-(5-(4-methoxyphenyl)-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (50 mg, 0.10 mmol) was reacted with 4-methoxybenzoyl chloride (19.2 μl, 0.14 mmol) to afford the intermediate compound 5-(2,4-bis(benzyloxy)-5-isopropylphenyl)-N-ethyl-4-(5-(4-methoxyphenyl)-1,2,4-oxadiazol-3-yl)isoxazole-3-carboxamide (29 mg, 0.05 mmol) in a yield of 47%.

¹H-NMR (400 MHz, CDCl₃) δ 7.98 (dd, 2H), 7.52 (s, 1H), 7.38-7.30 (m, 5H), 7.24-7.17 (m, 4H), 7.11 (m, 2H), 6.97 (dd, 2H), 6.43 (s, 1H), 4.96 (s, 2H), 4.86 (s, 2H), 3.88 (s, 3H), 3.48 (m, 2H), 3.28 (sept, 1H), 1.24 (t, 3H), 1.19 (d, 6H)

### Step 2

### 5-(2,4-Dihydroxy-5-isopropylphenyl)-N-ethyl-4-(5-(4-methoxyphenyl)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (16.5 mg, 0.04 mmol) in a yield of 79%.

¹H-NMR (400 MHz, CD₃OD) δ 8.08 (dd, 2H), 7.33 (s, 1H), 7.10 (m, 2H), 6.31 (s, 1H), 3.90 (s, 3H), 3.41 (q, 2H), 3.18 (sept, 1H), 1.24 (t, 3H), 1.17 (d, 6H)

### Example 85

### 5-(2,4-Dihydroxy-5-isopropylphenyl)-N-ethyl-4-(5-(4-nitrophenyl)-1,2,4-oxadiazol-3-yl)isoxazole-3-carboxamide(I-85)

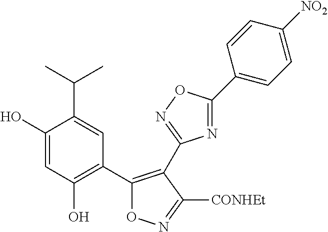

### Step 1

### 5-(2,4-Bis(benzyloxy)-5-isopropylphenyl)-N-ethyl-4-(5-(4-nitrophenyl-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (80 mg, 0.15 mmol) was reacted with 4-nitrobenzoyl chloride (42 mg, 0.23 mmol) to afford the intermediate compound 5-(2,4-bis(benzyloxy)-5-isopropylphenyl)-N-ethyl-4-(5-(4-nitrophenyl-1,2,4-oxadiazol-3-yl)isoxazole-3-carboxamide (80 mg, 0.12 mmol) in a yield of 80%.

¹H-NMR (400 MHz, CDCl₃) δ 8.31 (m, 2H), 8.16 (m, 2H), 7.55 (s, 1H), 7.38-7.30 (m, 5H), 7.17-7.14 (m, 3H), 7.04 (m, 2H), 6.90 (br t, 1H), 6.42 (s, 1H), 4.97 (s, 2H), 4.84 (s, 2H), 3.48 (m, 2H), 3.30 (sept, 1H), 1.26 (t, 3H), 1.21 (d, 6H)

### Step 2

### 5-(2,4-Dihydroxy-5-isopropylphenyl)-N-ethyl-4-(5-(4-nitrophenyl)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (45 mg, 0.09 mmol) in a yield of 77%.

¹H-NMR (400 MHz, CD₃OD) δ 8.45 (m, 2H), 8.38 (m, 2H), 7.35 (s, 1H), 6.30 (s, 1H), 3.40 (q, 2H), 3.19 (sept, 1H), 1.24 (t, 3H), 1.19 (d, 6H)

### Example 86

### 5-(2,4-Dihydroxy-5-isopropylphenyl)-N-ethyl-4-(5-methoxy-1,2,4-oxadiazol-3-yl)isoxazole-3-carboxamide (I-86)

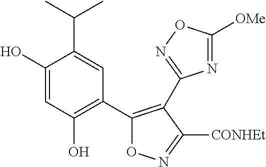

### Step 1

### 5-(2,4-Bis(benzyloxy)-5-isopropylphenyl)-N-ethyl-4-(5-methoxy-1,2,4-oxadiazol-3-yl)isoxazole-3-carboxamide

5-(2,4-bis(Benzyloxy)-5-isopropylphenyl)-N-ethyl-4-(5-hydroxy-1,2,4-oxadiazol-3-yl)isoxazole-3-carboxamide (65 mg, 0.12 mmol) was dissolved in DMF (1 ml), potassium carbonate (40.5 mg, 0.29 mmol) and iodomethane (18.2 μl, 0.29 mmol) were added to this solution sequentially. This reaction mixture was stirred at RT for overnight, and extracted between ethyl acetate and water. The organic phase was washed with 2N—HCl, saturated aqueous NaCl, dried with magnesium sulfate, and evaporated in vacuo. The residue was purified by silica gel column chromatography to afford the intermediate compound 5-(2,4-bis(benzyloxy)-5-isopropylphenyl)-N-ethyl-4-(5-methoxy-1,2,4-oxadiazol-3-yl)isoxazole-3-carboxamide (55 mg, 0.10 mmol) in a yield of 82%.

¹H-NMR (400 MHz, CDCl₃) δ 7.52 (s, 1H), 7.40-7.32 (m, 8H), 7.18 (dd, 2H), 6.83 (br t, 1H), 6.48 (s, 1H), 4.99 (s, 2H), 4.92 (s, 2H), 3.45 (m, 2H), 3.30 (sept, 1H), 2.76 (s, 3H), 1.25 (t, 3H), 1.23 (d, 6H)

### Step 2

### 5-(2,4-Dihydroxy-5-isopropylphenyl)-N-ethyl-4-(5-methoxy-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (37 mg, 0.10 mmol) in a yield of 98%.

¹H-NMR (400 MHz, CD₃OD) δ 7.45 (s, 1H), 6.34 (s, 1H), 3.38 (q, 2H), 3.20 (sept, 1H), 3.15 (s, 3H), 1.22 (d, 6H), 1.21 (t, 3H)

### Example 87

### 5-(2,4-Dihydroxy-5-isopropylphenyl)-N-ethyl-4-(5-(methylthio)-1,2,4-oxadiazol-3-yl)isoxazole-3-carboxamide (I-87)

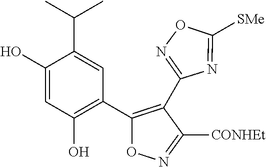

### Step 1

### 5-(2,4-Bis(benzyloxy)-5-isopropylphenyl)-N-ethyl-4-(5-(methylthio)-1,2,4-oxadiazol-3-yl)isoxazole-3-carboxamide

This compound was made using the procedure described for example 86 (Step 1). Thus, 5-(2,4-bis(benzyloxy)-5-isopropylphenyl)-N-ethyl-4-(5-mercapto-1,2,4-oxadiazol-3-yl)isoxazole-3-carboxamide (60 mg, 0.10 mmol) was reacted with iodomethane (16.4 μl, 0.26 mmol) to afford the intermediate compound 5-(2,4-bis(benzyloxy)-5-isopropylphenyl)-N-ethyl-4-(5-(methylthio)-1,2,4-oxad iazol-3-yl)isoxazole-3-carboxamide (45 mg, 0.08 mmol) in a yield of 73%.

¹H-NMR (400 MHz, CDCl₃) δ 7.46 (s, 1H), 7.39-7.28 (m, 8H), 7.18 (dd, 2H), 6.97 (br t, 1H), 6.42 (s, 1H), 4.96 (s, 2H), 4.90 (s, 2H), 3.47 (m, 2H), 3.28 (sept, 1H), 2.60 (s, 3H), 1.25 (t, 3H), 1.20 (d, 6H)

### Step 2

### 5-(2,4-Dihydroxy-5-isopropylphenyl)-N-ethyl-4-(5-(methylthio)-1,2,4-oxadiazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (22 mg, 0.05 mmol) in a yield of 71%.

¹H-NMR (400 MHz, CD₃OD) δ 7.27 (s, 1H), 6.32 (s, 1H), 3.39 (q, 2H), 3.18 (sept, 1H), 2.73 (s, 3H), 1.22 (t, 3H), 1.18 (d, 6H)

### Example 88

### 4-(5-Cyclopentyl-1,2,4-oxadiazol-3-yl)-5-(2,4-dihydroxy-5-isopropylphenyl)-N-ethylisoxazole-3-carboxamide (I-88)

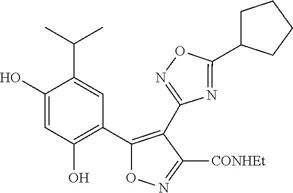

### Step 1

### 5-(2,4-Bis(benzyloxy)-5-isopropylphenyl)-4-(5-cyclopentyl-1,2,4-oxadiazol-3-yl)-N-ethylisoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (60 mg, 0.11 mmol) was reacted with cyclopentanecarbonyl chloride (20.7 μl, 0.17 mmol) to afford the intermediate compound 5-(2,4-bis(benzyloxy)-5-isopropylphenyl)-4-(5-cyclopentyl-1,2,4-oxadiazol-3-yl)-N-ethylisoxazole-3-carboxamide (41 mg, 0.07 mmol) in a yield of 60%.

¹H-NMR (400 MHz, CDCl₃) δ 7.43 (s, 1H), 7.37-7.27 (m, 8H), 7.18-7.16 (m, 3H), 6.41 (s, 1H), 4.95 (s, 2H), 4.90 (s, 2H), 3.47 (m, 2H), 3.29-3.23 (m, 2H), 2.06-2.04 (m, 2H), 1.86-1.63 (m, 6H), 1.25 (t, 3H), 1.18 (d, 6H)

### Step 2

### 4-(5-Cyclopentyl-1,2,4-oxadiazol-3-yl)-5-(2,4-dihydroxy-5-isopropylphenyl)-N-ethylisoxazole-3-carboxamide

This compound was made using the procedure described for example 1 (Step 3). Thus, this intermediate compound (Step 1) (38 mg, 0.06 mmol) was reacted with BCl₃ to afford a crude product, which was purified by silica gel column chromatography to afford the title compound (23 mg, 0.05 mmol) in a yield of 86%.

¹H-NMR (400 MHz, CD₃OD) δ 7.26 (s, 1H), 6.32 (s, 1H), 3.44-3.34 (m, 3H), 3.17 (sept, 1H), 2.17-2.11 (m, 2H), 1.97-1.90 (m, 2H), 1.84-1.71 (m, 4H), 1.22 (t, 3H), 1.18 (d, 6H)

### Example 89

### 4-(5-Cyclohexyl-1,2,4-oxadiazol-3-yl)-5-(2,4-dihydroxy-5-isopropylphenyl)-N-ethylisoxazole-3-carboxamide (I-89)

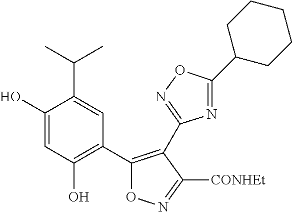

### Step 1

### 5-(2,4-Bis(benzyloxy)-5-isopropylphenyl)-4-(5-cyclohexyl-1,2,4-oxadiazol-3-yl)-N-ethylisoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (60 mg, 0.11 mmol) was reacted with cyclohexanecarbonyl chloride (22.8 μl, 0.17 mmol) to afford the intermediate compound 5-(2,4-bis(benzyloxy)-5-isopropylphenyl)-4-(5-cyclohexyl-1,2,4-oxadiazol-3-yl)-N-ethylisoxazole-3-carboxamide (42 mg, 0.07 mmol) in a yield of 60%.

¹H-NMR (400 MHz, CDCl₃) δ 7.44 (s, 1H), 7.39-7.27 (m, 8H), 7.19-7.15 (m, 3H), 6.41 (s, 1H), 4.95 (s, 2H), 4.90 (s, 2H), 3.47 (m, 2H), 3.28 (sept, 1H), 2.83 (m, 1H), 2.03-2.00 (m, 2H), 1.81-1.76 (m, 2H), 1.68 (m, 1H), 1.56-1.46 (m, 2H), 1.39-1.27 (m, 3H), 1.25 (t, 3H), 1.19 (d, 6H)

### Step 2

### 4-(5-Cyclohexyl-1,2,4-oxadiazol-3-yl)-5-(2,4-dihydroxy-5-isopropylphenyl)-N-ethylisoxazole-3-carboxamide

This compound was made using the procedure described for example 1 (Step 3). Thus, this intermediate compound (Step 1) (39 mg, 0.06 mmol) was reacted with BCl₃ to afford a crude product, which was purified by silica gel column chromatography to afford the title compound (21 mg, 0.05 mmol) in a yield of 76%.

¹H-NMR (400 MHz, CD₃OD) δ 7.27 (s, 1H), 6.32 (s, 1H), 3.39 (q, 2H), 3.17 (sept, 1H), 3.02 (m, 1H), 2.11-2.07 (m, 2H), 1.85-1.81 (m, 2H), 1.74-1.61 (m, 3H), 1.50-1.29 (m, 3H), 1.22 (t, 3H), 1.18 (d, 6H)

### Example 90

### 5-(2,4-Dihydroxy-5-isopropylphenyl)-4-(5-(4-ethoxyphenyl)-1,2,4-oxadiazol-3-1)-N-ethylisoxazole-3-carboxamide (I-90)

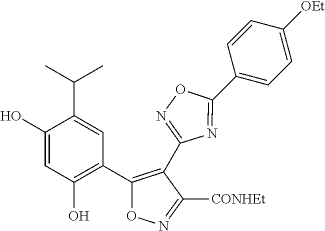

### Step 1

### 5-(2,4-Bis(benzloxy-isopropylphenyl)-4-(5-(4-ethoxyphenyl)-1,2,4-oxadiazol-3-yl)-N-ethylisoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (60 mg, 0.11 mmol) was reacted with 4-ethoxybenzoyl chloride (31.4 mg, 0.17 mmol) and pyridine (30) to afford the intermediate compound 5-(2,4-bis(benzyloxy)-5-isopropylphenyl)-4-(5-(4-ethoxyphenyl)-1,2,4-oxadiazol-3-yl)-N-ethylisoxazole-3-carboxamide (55 mg, 0.08 mmol) in a yield of 74%.

¹H-NMR (400 MHz, CDCl₃) δ 7.97 (dd, 2H), 7.51 (s, 1H), 7.38-7.31 (m, 5H), 7.23-7.17 (m, 4H), 7.11 (m, 2H), 6.95 (dd, 2H), 6.43 (s, 1H), 4.96 (s, 2H), 4.86 (s, 2H), 4.11 (q, 2H), 3.48 (m, 2H), 3.28 (sept, 1H), 1.46 (t, 3H), 1.25 (t, 3H), 1.19 (d, 6H)

### Step 2

### 5-(2,4-Dihydroxy-5-isopropylphenyl)-4-(5-(4-ethoxyphenyl)-1,2,4-oxadiazol-3-yl)-N-ethylisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (25 mg, 0.05 mmol) in a yield of 63%.

¹H-NMR (400 MHz, CD₃OD) δ 8.06 (m, 2H), 7.32 (s, 1H), 7.08 (m, 2H), 6.31 (s, 1H), 4.14 (q, 2H), 3.40 (q, 2H), 3.17 (sept, 1H), 1.43 (t, 3H), 1.23 (t, 3H), 1.17 (d, 6H)

### Example 91

### 4-(5-(2-Chloro-1-hydroxyethyl)-1,2,4-oxadiazol-3-yl)-5-(2,4-dihydroxy-5-isopropylphenyl)-N-ethylisoxazole-3-carboxamide (I-91)

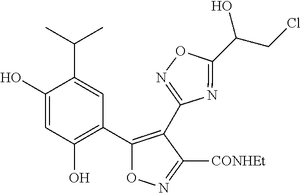

### Step 1

### 5-(2,4-Bis(benzyloxy)-5-isopropylphenyl)-N-ethyl-4-(5-vinyl-1,2,4-oxadiazol-3-yl)isoxazole-3-carboxamide

This compound was made using the procedure described for example 57 (Step 3). Thus, 5-(2,4-bis(benzyloxy)-5-isopropylphenyl)-N-ethyl-4-(N′-hydroxycarbamimidoyl)isoxazole-3-carboxamide (150 mg, 0.28 mmol) was reacted with pyridine (69 μl, 0.85 mmol) and acryloyl chloride (34.6 μl, 0.43 mmol) to afford the intermediate compound 5-(2,4-bis(benzyloxy)-5-isopropylphenyl)-N-ethyl-4-(5-vinyl-1,2,4-oxadiazol-3-yl)isoxazole-3-carboxamide (82 mg, 0.14 mmol) in a yield of 51%.

¹H-NMR (400 MHz, CDCl₃) δ 7.47 (s, 1H), 7.39-7.26 (m, 8H), 7.14 (dd, 2H), 7.00 (br t, 1H), 6.62 (dd, 1H), 6.45-6.41 (m, 2H), 5.89 (dd, 1H), 4.96 (s, 2H), 4.87 (s, 2H), 3.47 (m, 2H), 3.28 (sept, 1H), 1.25 (t, 3H), 1.19 (d, 6H)

### Step 2

### 5-(2,4-Bis(benzyloxy)-5-isopropylphenyl)-N-ethyl-4-(5-(oxiran-2-yl)-1,2,4-oxadiazol-3-yl)isoxazole-3-carboxamide

This intermediate compound (Step 1) (40 mg, 0.07 mmol) and benzonitrile (11 μl, 0.11 mmol) were dissolved in MeOH (1 ml). KHCO₃ (7.1 mg, 0.07 mmol) and hydrogen peroxide (11 μl, 0.11 mmol) were added. This mixture was stirred at RT for overnight, evaporated in vacuo. The residue was purified by silica gel column chromatography to afford the intermediate compound 5-(2,4-bis(benzyloxy)-5-isopropylphenyl)-N-ethyl-4-(5-(oxiran-2-yl)-1,2,4-oxa diazol-3-yl)isoxazole-3-carboxamide (20 mg, 0.03 mmol) in a yield of 49%.

¹H-NMR (400 MHz, CDCl₃) δ 7.48 (s, 1H), 7.39-7.27 (m, 8H), 7.14 (dd, 2H), 6.92 (br t, 1H), 6.42 (s, 1H), 4.96 (s, 2H), 4.86 (s, 2H), 4.00 (dd, 1H), 3.46 (m, 2H), 3.29 (sept, 1H), 3.17 (m, 2H), 1.24 (t, 3H), 1.21 (d, 6H)

### Step 3

### 4-(5-(2-Chloro-1-hydroxyethyl)-1,2,4-oxadiazol-3-yl)-5-(2,4-dihydroxy-5-isopropylphenyl)-N-ethylisoxazole-3-carboxamide

This compound was made using the procedure described for example 1 (Step 3). Thus, this intermediate compound (Step 2) (20 mg, 0.03 mmol) was reacted with BCl₃ to afford a crude product, which was purified by silica gel column chromatography to afford the title compound (13 mg, 0.03 mmol) in a yield of 86%.

¹H-NMR (400 MHz, CD₃OD) δ 7.28 (s, 1H), 6.32 (s, 1H), 5.14 (dd, 1H), 3.96 (dd, 1H), 3.89 (dd, 1H), 3.39 (q, 2H), 3.18 (sept, 1H), 1.23 (t, 3H), 1.19 (d, 6H)

### Example 92

### 5-(2,4-Dihydroxy-5-isopropylphenyl)-N-ethyl-4-(5-(morpholinomethyl)-1,2,4-oxadiazol-3-yl)isoxazole-3-carboxamide (I-92)

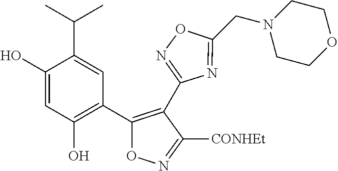

### Step 1

### (3-(5-(2,4-Bis(benzyloxy)-5-isopropylphenyl)-3(ethylcarbamoyl)isoxazol-4-yl)-1,2,4-oxadiazol-5-yl)methyl methanesulfonate

This compound was made using the procedure described for example 18 (Step 1). Thus, 5-(2,4-bis(benzyloxy)-5-isopropylphenyl)-N-ethyl-4-(5-(hydroxymethyl)-1,2,4-oxadiazol-3-yl)isoxazole-3-carboxamide (143 mg, 0.25 mmol) was reacted with methanesulfonyl chloride (39 μl, 0.50 mmol) to afford the intermediate compound (3-(5-(2,4-bis(benzyloxy)-5-isopropylphenyl)-3-(ethylcarbamoyl)isoxazol-4-yl)-1,2,4-oxadiazol-5-yl)methyl methanesulfonate (146 mg, 0.23 mmol) in a yield of 90%.

¹H-NMR (400 MHz, CDCl₃) δ 7.50 (s, 1H), 7.40-7.28 (m, 8H), 7.11 (dd, 2H), 6.83 (br t, 1H), 6.43 (s, 1H), 5.18 (s, 2H), 4.99 (s, 2H), 4.86 (s, 2H), 3.46 (m, 2H), 3.31 (sept, 1H), 3.01 (s, 3H), 1.25 (t, 3H), 1.22 (d, 6H)

### Step 2

### 5-(2,4-Bis(benzyloxy)-5-isopropylphenyl)-N-ethyl-4-(5(morpholinomethyl)-1,2,4-oxadiazol-3-yl)isoxazole-3-carboxamide

This compound was made using the procedure described for example 18 (Step 2). Thus, this intermediate compound (Step 1) (69 mg, 0.11 mmol) was reacted with morpholine (37.3 μl, 0.43 mmol) to afford the intermediate compound 5-(2,4-bis(benzyloxy)-5-isopropylphenyl)-N-ethyl-4-(5-(morpholinomethyl)-1,2,4-oxadiazol-3-yl)isoxazole-3-carboxamide (67 mg, 0.10 mmol) in a yield of 98%.

¹H-NMR (400 MHz, CDCl₃) δ 7.46 (s, 1H), 7.39-7.28 (m, 8H), 7.15 (dd, 2H), 6.95 (br t, 1H), 6.42 (s, 1H), 4.96 (s, 2H), 4.89 (s, 2H), 3.70 (s, 2H), 3.65 (t, 4H), 3.47 (m, 2H), 3.29 (sept, 1H), 2.53 (t, 4H), 1.25 (t, 3H), 1.20 (d, 6H)

### Step 3

### 5-(2,4-Dihydroxy-5-isopropylphenyl)-N-ethyl-4-(5-(morpholinomethyl)-1,2,4-oxadiazol-3-yl)isoxazole-3-carboxamide

This compound was made using the procedure described for example 1 (Step 3). Thus, this intermediate compound (Step 2) was reacted with BCl₃ to afford a crude product, which was purified by silica gel column chromatography to afford the title compound (48 mg, 0.10 mmol) in a yield of 99%.

¹H-NMR (400 MHz, CD₃OD) δ 7.30 (s, 1H), 6.30 (s, 1H), 3.89 (s, 2H), 3.70 (t, 4H), 3.39 (q, 2H), 3.18 (sept, 1H), 2.60 (t, 4H), 1.22 (t, 3H), 1.20 (d, 6H)

### Example 93

### 5-(2,4-Dihydroxy-5-isopropylphenyl)-4-(5-((dimethylamino)methyl)-1,2,4-oxadiazol-3-yl)-N-ethylisoxazole-3-carboxamide (I-93)

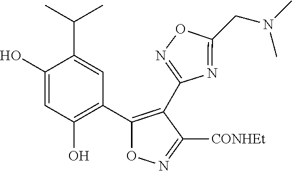

### Step 1

### 5-(2,4-Bis(benzyloxy)-5-isopropylphenyl)-4-(5-((dimethylamino)methyl)-1,2,4-oxadiazol-3-yl)-N-ethylisoxazole-3-carboxamide

This compound was made using the procedure described for example 18 (Step 2). Thus, (3-(5-(2,4-bis(benzyloxy)-5-isopropylphenyl)-3-(ethylcarbamoyl) isoxazol-4-yl)-1,2,4-oxadiazol-5-yl)methyl methanesulfonate (67 mg, 0.10 mmol) was reacted with aqueous 50% dimethylamine (0.5 ml) to afford the intermediate compound 5-(2,4-bis(benzyloxy)-5-isopropylphenyl)-4-(5-((dimethylamino)methyl)-1,2,4-oxadiazol-3-yl)-N-ethylisoxazole-3-carboxamide (56 mg, 0.09 mmol) in a yield of 91%.

¹H-NMR (400 MHz, CDCl₃) δ 7.46 (s, 1H), 7.39-7.28 (m, 8H), 7.16 (dd, 2H), 6.99 (br t, 1H), 6.43 (s, 1H), 4.97 (s, 2H), 4.89 (s, 2H), 3.67 (s, 2H), 3.47 (m, 2H), 3.28 (sept, 1H), 2.32 (s, 6H), 1.25 (t, 3H), 1.19 (d, 6H)

### Step 2

### 5-(2,4-Dihydroxy-5-isopropylphenyl)-4-(5-((dimethylamino)methyl)-1,2,4-oxadiazol-3-yl)-N-ethylisoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (24 mg, 0.06 mmol) in a yield of 61%.

¹H-NMR (400 MHz, CD₃OD) δ 7.31 (s, 1H), 6.31 (s, 1H), 3.87 (s, 2H), 3.39 (q, 2H), 3.18 (sept, 1H), 2.37 (s, 6H), 1.24 (t, 3H), 1.19 (d, 6H)

### Example 94

### 5-(2,4-Dihydroxy-5-isopropylphenyl)-N-ethyl-4-(5-(piperidin-1-ylmethyl)-1,2,4-oxadiazol-3-yl)isoxazole-3-carboxamide (I-94)

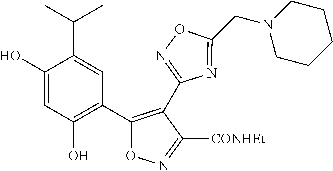

### Step 1

### 5-(2,4-Bis(benzyloxy)-5-isopropylphenyl)-N-ethyl-4-(5-(piperidin-1-ylmethyl)-1,2,4-oxadiazol-3-yl)isoxazole-3-carboxamide

This compound was made using the procedure described for example 18 (Step 2). Thus, (3-(5-(2,4-bis(benzyloxy)-5-isopropylphenyl)-3-(ethylcarbamoyl) isoxazol-4-yl)-1,2,4-oxadiazol-5-yl)methyl methanesulfonate (73 mg, 0.11 mmol) was reacted with piperidine (55.8 μl, 0.56 mmol) to afford the intermediate compound 5-(2,4-bis(benzyloxy)-5-isopropylphenyl)-N-ethyl-4-(5-(piperidin-1-ylmethyl)-1,2,4-oxadiazol-3-yl)isoxazole-3-carboxamide (69 mg, 0.11 mmol) in a yield of 96%.

¹H-NMR (400 MHz, CDCl₃) δ 7.45 (s, 1H), 7.40-7.27 (m, 8H), 7.16 (dd, 2H), 7.04 (br t, 1H), 6.42 (s, 1H), 4.96 (s, 2H), 4.89 (s, 2H), 3.71 (s, 2H), 3.47 (m, 2H), 3.28 (sept, 1H), 2.47 (t, 4H), 1.55 (m, 4H), 1.39 (m, 2H), 1.25 (t, 3H), 1.19 (d, 6H)

### Step 2

### 5-(2,4-Dihydroxy-5-isopropylphenyl)-N-ethyl-4-(5-(piperidin-1-ylmethyl)-1,2,4-oxadiazol-3-yl)isoxazole-3-carboxamide

This compound was made using the procedure described for example 1 (Step 3). Thus, this intermediate compound (Step 1) (66 mg, 0.10 mmol) was reacted with BCl₃ to afford a crude product, which was purified by silica gel column chromatography to afford the title compound (47 mg, 0.10 mmol) in a yield of 99%.

¹H-NMR (400 MHz, CD₃OD) δ 7.29 (s, 1H), 6.31 (s, 1H), 3.85 (s, 2H), 3.39 (q, 2H), 3.18 (sept, 1H), 2.56 (t, 4H), 1.62 (quint, 4H), 1.47 (m, 2H), 1.22 (t, 3H), 1.19 (d, 6H)

### Example 95

### 5-(2,4-Dihydroxy-5-isopropylphenyl)-N-ethyl-4-(5-(pyrrolidin-1-ylmethyl)-1,2,4-oxadiazol-3-yl)isoxazole-3-carboxamide (I-95)

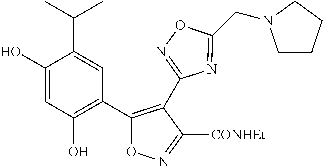

### Step 1

### 5-(2,4-Bis(benzyloxy)-5-isopropylphenyl)-N-ethyl-4-(5-(pyrrolidin-1-ylmethyl)-1,2,4-oxadiazol-3-yl)isoxazole-3-carboxamide

This compound was made using the procedure described for example 18 (Step 2). Thus, (3-(5-(2,4-bis(benzyloxy)-5-isopropylphenyl)-3-(ethylcarbamoyl) isoxazol-4-yl)-1,2,4-oxadiazol-5-yl)methyl methanesulfonate (73 mg, 0.11 mmol) was reacted with pyrrolidine (47.1 μl, 0.56 mmol) to afford the intermediate compound 5-(2,4-bis(benzyloxy)-5-isopropylphenyl)-N-ethyl-4-(5-(pyrrolidin-1-ylmethyl)-1,2,4-oxadiazol-3-yl)isoxazole-3-carboxamide (65 mg, 0.10 mmol) in a yield of 93%.

¹H-NMR (400 MHz, CDCl₃) δ 7.45 (s, 1H), 7.39-7.27 (m, 8H), 7.16 (dd, 2H), 7.02 (br t, 1H), 6.42 (s, 1H), 4.96 (s, 2H), 4.89 (s, 2H), 3.85 (s, 2H), 3.48 (m, 2H), 3.28 (sept, 1H), 2.62 (m, 4H), 1.76 (m, 4H), 1.25 (t, 3H), 1.19 (d, 6H)

### Step 2

### 5-(2,4-Dihydroxy-5-isopropylphenyl)-N-ethyl-4-(5-(pyrrolidin-1-ylmethyl)-1,2,4-oxadiazol-3-yl)isoxazole-3-carboxamide

This compound was made using the procedure described for example 1 (Step 3). Thus, this intermediate compound (Step 1) (62 mg, 0.10 mmol) was reacted with BCl₃ to afford a crude product, which was purified by silica gel column chromatography to afford the title compound (42.5 mg, 0.10 mmol) in a yield of 96%.

¹H-NMR (400 MHz, CD₃OD) δ 7.29 (s, 1H), 6.31 (s, 1H), 4.01 (s, 2H), 3.39 (q, 2H), 3.18 (sept, 1H), 2.71 (m, 4H), 1.84 (m, 4H), 1.22 (t, 3H), 1.19 (d, 6H)

### Example 96

### 5-(2,4-Dihydroxy-5-isopropylphenyl)-N-ethyl-4-(1H-pyrazol-3-yl)isoxazole-3-carboxamide (I-96)

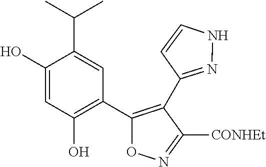

### Step 1

### 5-(2,4-Bis(benzyloxy)-5-isopropylphenyl)-N-ethyl-4-ethynyl isoxazole-3-carboxamide

A soluntion 5-(2,4-bis(benzyloxy)-5-isopropylphenyl)-N-ethyl-4-iodoisoxazole-3-carboxamide (1.5 g, 2.52 mmol) and tetrakis(triphenylphosphine)palladium(0) (145 mg, 0.13 mmol) in toluene (25 ml) heated at 112° C. After 10 min, tributyl(ethynyl)stannane (0.87 ml, 3.02 mmol) was added, and the suspension was heated to reflux for 2 h. The reaction mixture was cooled to ambient temperature, solvent was evaporated in vacuo. The residue was purified by silica gel column chromatography to afford the intermediate compound 5-(2,4-bis(benzyloxy)-5-isopropylphenyl)-N-ethyl-4-ethynyl isoxazole-3-carboxamide (934 mg, 1.89 mmol) in a yield of 75%.

¹H-NMR (400 MHz, CDCl₃) δ 7.76 (s, 1H), 7.42-7.29 (m, 10H), 6.78 (s, 1H), 6.58 (s, 1H), 5.12 (s, 2H), 5.06 (s, 2H), 3.51 (m, 2H), 3.32 (m, 1H), 1.26 (t, 3H), 1.19 (d, 6H)

### Step 2

### 4-Acetyl-5-(2,4-bis(benzyloxy)-5-isopropylphenyl)-N-ethylisoxazole-3-carboxamide

This intermediate (Step 1) compound was dissolved in formic acid (20 ml), and this solution was stirred at 95° C. under a nitrogen atmosphere. After 1 h, NaHCO₃ was added to the reaciton mixture until pH=8. Solvent was removed in vacuo, and the residue was extracted between methylene chloride and water. The organic phase was dried with magnesium sulfate, and evaporated in vacuo. The residue was purified by silica gel column chromatography to afford the intermediate compound 4-acetyl-5-(2,4-bis(benzyloxy)-5-isopropylphenyl)-N-ethylisoxazole-3-carboxamide (431 mg, 0.85 mmol) in a yield of 45%.

¹H-NMR (400 MHz, CDCl₃) δ 7.44-7.24 (m, 12H), 6.52 (s, 1H), 5.03 (s, 2H), 5.01 (s, 2H), 3.49 (m, 2H), 3.31 (m, 1H), 2.32 (s, 3H), 1.27 (t, 3H), 1.23 (d, 6H)

### Step 3

### (E)-5-(2,4-Bis(benzyloxy)-5-isopropylphenyl)-4-(3-(dimethylamino)acryloyl)-N-ethylisoxazole-3-carboxamide

This intermediate compound (Step 2) was dissolved in EtOH (8 ml) under a nitrogen atmosphere. N,N-dimethylformamide dimethylacetal (0.65 ml, 4.92 mmol) was added. This reaction mixture was heated to reflux for 4 h, stirred at RT for overnight, and evaporated in vacuo. The residue was purified by silica gel column chromatography to afford the intermediate compound (E)-5-(2,4-bis(benzyloxy)-5-isopropylphenyl)-4-(3-(dimethylamino)acryloyl)-N-ethylisoxazole-3-carboxamide (356 mg, 0.63 mmol) in a yield of 76%.

¹H-NMR (400 MHz, CDCl₃) δ 7.45-7.24 (m, 13H), 6.51 (s, 1H), 5.03 (s, 2H), 5.01 (s, 2H), 3.50 (m, 2H), 3.31 (m, 1H), 2.96 (s, 3H), 2.36 (s, 3H), 1.27 (t, 3H), 1.17 (d, 6H)

### Step 4

### 5-(2,4-Bis(benzyloxy)-5-isopropylphenyl)-N-ethyl-4-(1H-pyrazol-3-yl)isoxazole-3-carboxamide

This intermediate compound (Step 3) was dissolved in EtOH (7 ml), hydrazine monohydrate (12 μl, 2.47 mmol) was added. The reaction mixture was stirred at RT for 67 h. Methylene chloride was added to this reaction mixture, solvent was removed in vacuo to afford the intermediate compound 5-(2,4-bis(benzyloxy)-5-isopropylphenyl)-N-ethyl-4-(1H-pyrazol-3-yl)isoxazole-3-carboxamide (331 mg, 0.62 mmol) in a yield of 99%.

¹H-NMR (400 MHz, CDCl₃) δ 13.26 (br, 1H), 7.46-7.36 (m, 5H), 7.30-7.20 (m, 6H), 7.08-7.05 (m, 2H), 6.62 (s, 1H), 5.95 (d, 1H), 5.09 (s, 2H), 4.94 (s, 2H), 3.56 (m, 2H), 3.34 (m, 1H), 1.30 (t, 3H), 1.19 (d, 6H)

### Step 5

### 5-(2,4-Dihydroxy-5-isopropylphenyl)-N-ethyl-4-(1H-pyrazol-3-yl)isoxazole-3-carboxamide

This compound was made using the procedure described for example 1 (Step 3). Thus, this intermediate compound (Step 4) (180 mg, 0.34 mmol) was reacted with BCl₃ to afford a crude product, which was purified by silica gel column chromatography to afford the title compound (80.0 mg, 0.22 mmol) in a yield of 67%.

¹H-NMR (400 MHz, CD₃OD) δ 7.55 (br, 1H), 6.77 (s, 1H), 6.41 (s, 1H), 6.19 (br, 1H) 3.43 (m, 2H), 3.17 (m, 1H), 1.24 (t, 3H), 1.12 (d, 6H)

### Example 97

### 5-(2,4-Dihydroxy-5-isopropylphenyl)-N-ethyl-4-(1-methyl-1H-pyrazol-3-yl)isoxazole-3-carboxamide (I-97)

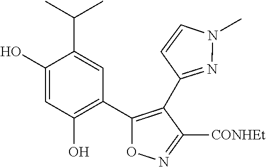

### Step 1

### 5-(2,4-Bis(benzyloxy)-5-isopropylphenyl)-N-ethyl-4-(1-methyl-1H-pyrazol-3-yl)isoxazole-3-carboxamide

This compound was made using the procedure described for example 11 (Step 1). Thus, 5-(2,4-bis(benzyloxy)-5-isopropylphenyl)-N-ethyl-4-(1H-pyrazol-3-yl)isoxazole-3-carboxamide (80 mg, 0.15 mmol) was reacted with iodomethane (11 μl, 0.18 mmol) to afford the intermediate compound 5-(2,4-bis(benzyloxy)-5-isopropylphenyl)-N-ethyl-4-(1-methyl-1H-pyrazol-3-yl) isoxazole-3-carboxamide (67.8 mg, 0.12 mmol) in a yield of 83%.

¹H-NMR (400 MHz, CDCl₃) δ 8.54 (br, 1H), 7.42-7.20 (m, 14H), 6.51 (s, 1H), 6.12 (d, 1H), 5.02 (s, 2H), 4.92 (s, 2H), 3.85 (s, 3H), 3.50 (m, 2H), 3.28 (m, 1H), 1.26 (t, 3H), 1.14 (d, 6H)

### Step 2

### 5-(2,4-Dihydroxy-5-isopropylphenyl)-N-ethyl-4-(1-methyl-1H-pyrazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (43.7 mg, 0.12 mmol) in a yield of 99%.

¹H-NMR (400 MHz, CD₃OD) δ 7.52 (d, 1H), 7.01 (s, 1H), 6.40 (s, 1H), 6.20 (s, 1H), 3.89 (s, 3H), 3.42 (m, 2H), 3.15 (m, 1H), 1.25 (t, 3H), 1.10 (d, 6H)

### Example 98

### 5-(2,4-Dihydroxy-5-isopropylphenyl)-N-ethyl-4-(1-methyl-1H-pyrazol-5-yl)isoxazole-3-carboxamide (I-98)

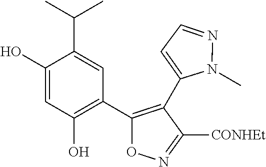

### Step 1

### 5-(2,4-Bis(benzyloxy)-5-isopropylphenyl)-N-ethyl-4-(1-methyl-1H-pyrazol-5-yl)isoxazole-3-carboxamide

This compound was made using the procedure described for example 11 (Step 1). Thus, 5-(2,4-bis(benzyloxy)-5-isopropylphenyl)-N-ethyl-4-(1H-pyrazol-3-yl)isoxazole-3-carboxamide (80 mg, 0.15 mmol) was reacted with iodomethane (11 μl, 0.18 mmol) to afford the intermediate compound 5-(2,4-bis(benzyloxy)-5-isopropylphenyl)-N-ethyl-4-(1-methyl-1H-pyrazol-5-yl) isoxazole-3-carboxamide (4.9 mg, 0.009 mmol) in a yield of 6%.

¹H-NMR (400 MHz, CDCl₃) δ 7.46 (s, 1H), 7.45-7.28 (m, 14H), 6.76 (t, 1H), 6.43 (s, 1H), 6.16 (d, 1H), 4.95 (s, 2H), 4.84 (s, 2H), 3.52 (s, 3H), 3.44 (m, 2H), 3.24 (m, 1H), 1.24 (t, 3H), 1.11 (d, 6H)

### Step 2

### 5-(2,4-Dihydroxy-5-isopropylphenyl)-N-ethyl-4-(1-methyl-1H-pyrazol-5-yl)isoxazole-3-carboxamide

This compound was made using the procedure described for example 1 (Step 3). Thus, this intermediate compound (Step 1) (181.4 mg, 0.329 mmol) was reacted with BCl₃ to afford a crude product, which was purified by silica gel column chromatography to afford the title compound (40.0 mg, 0.11 mmol) in a yield of 33%.

¹H-NMR (400 MHz, CD₃OD) δ 7.55 (d, 1H), 6.98 (s, 1H), 6.30 (s, 1H), 6.25 (s, 1H), 3.65 (s, 3H), 3.33 (m, 2H), 3.10 (m, 1H), 1.18 (t, 3H), 1.14 (d, 6H)

### Example 99

### 5-(2,4-Dihydroxy-5-isopropylphenyl)-N-ethyl-4-(1-ethyl-1H-pyrazol-3-yl)isoxazole-3-carboxamide (I-99)

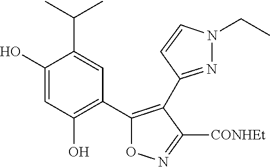

### Step 1

### 5-(2,4-Bis(benzyloxy)-5-isopropylphenyl)-N-ethyl-4-(1-ethyl-1H-pyrazol-3-yl)isoxazole-3-carboxamide

This compound was made using the procedure described for example 11 (Step 1). Thus, 5-(2,4-bis(benzyloxy)-5-isopropylphenyl)-N-ethyl-4-(1H-pyrazol-3-yl)isoxazole-3-carboxamide (100 mg, 0.19 mmol) was reacted with iodoethane (36 μl, 0.45 mmol) to afford the intermediate compound 5-(2,4-bis(benzyloxy)-5-isopropylphenyl)-N-ethyl-4-(1-ethyl-1H-pyrazol-3-yl)isoxazole-3-carboxamide (91.2 mg, 0.16 mmol) in a yield of 87%.

¹H-NMR (400 MHz, CDCl₃) δ 8.76 (s, 1H), 7.42-7.19 (m, 14H), 6.52 (s, 1H), 6.09 (d, 1H), 5.02 (s, 2H), 4.92 (s, 2H), 4.12 (q, 2H), 3.50 (m, 2H), 3.28 (m, 1H), 1.46 (t, 3H), 1.26 (t, 3H), 1.13 (d, 6H)

### Step 2

### 5-(2,4-Dihydroxy-5-isopropylphenyl)-N-ethyl-4-(1-ethyl-1H-pyrazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (57.5 mg, 0.15 mmol) in a yield of 95%.

¹H-NMR (400 MHz, CD₃OD) δ 7.59 (s, 1H), 7.00 (s, 1H), 6.39 (s, 1H), 6.20 (s, 1H), 4.18 (q, 2H), 3.42 (q, 2H), 3.14 (m, 1H), 1.45 (t, 3H), 1.23 (t, 3H), 1.10 (d, 6H)

### Example 100

### 5-(2,4-Dihydroxy-5-isopropylphenyl)-N-ethyl-4-(1-isopropyl-1H-pyrazol-3-yl)isoxazole-3-carboxamide (I-100)

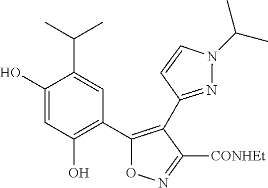

### Step 1

### 5-(2,4-Bis(benzyloxy)-5-isopropylphenyl)-N-ethyl-4-(1-isopropyl-1H-pyrazol-3-yl)isoxazole-3-carboxamide

This compound was made using the procedure described for example 11 (Step 1). Thus, 5-(2,4-bis(benzyloxy)-5-isopropylphenyl)-N-ethyl-4-(1H-pyrazol-3-yl)isoxazole-3-carboxamide (100 mg, 0.19 mmol) was reacted with 2-iodopropane (45 μl, 0.45 mmol) to afford the intermediate compound 5-(2,4-bis(benzyloxy)-5-isopropylphenyl)-N-ethyl-4-(1-isopropyl-1H-pyrazol-3-yl)isoxazole-3-carboxamide (84.1 mg, 0.15 mmol) in a yield of 78%.

¹H-NMR (400 MHz, CDCl₃) δ 9.02 (s, 1H), 7.42-7.19 (m, 14H), 6.52 (s, 1H), 6.05 (d, 1H), 5.02 (s, 2H), 4.92 (s, 2H), 4.45 (m, 1H), 3.51 (m, 2H), 3.27 (m, 1H), 1.48 (d, 6H), 1.26 (t, 3H), 1.13 (d, 6H)

### Step 2

### 5-(2,4-Dihydroxy-5-isopropylphenyl)-N-ethyl-4-(1-isopropyl-1H-pyrazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (53.1 mg, 0.14 mmol) in a yield of 99%.

¹H-NMR (400 MHz, CD₃OD) δ 7.62 (s, 1H), 7.00 (s, 1H), 6.41 (s, 1H), 6.21 (d, 1H), 5.53 (m, 1H), 3.42 (m, 2H), 3.15 (m, 1H), 1.49 (d, 6H), 1.24 (t, 3H), 1.11 (d, 6H)

### Example 101

### 5-(2,4-Dihydroxy-5-isopropylphenyl)-N-ethyl-4-(1-propyl-1H-pyrazol-3-yl)isoxazole-3-carboxamide (I-101)

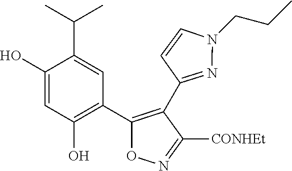

### Step 1

### 5-(2,4-Bis(benzyloxy)-5-isopropylphenyl)-N-ethyl-4-(1-propyl-1H-pyrazol-3-yl)isoxazole-3-carboxamide

This compound was made using the procedure described for example 11 (Step 1). Thus, 5-(2,4-bis(benzyloxy)-5-isopropylphenyl)-N-ethyl-4-(1H-pyrazol-3-yl)isoxazole-3-carboxamide (80 mg, 0.15 mmol) was reacted with 1-iodopropane (73 μl, 0.75 mmol) to afford the intermediate compound 5-(2,4-bis(benzyloxy)-5-isopropylphenyl)-N-ethyl-4-(1-propyl-1H-pyrazol-3-yl) isoxazole-3-carboxamide (75.0 mg, 0.13 mmol) in a yield of 87%.

¹H-NMR (400 MHz, CDCl₃) δ 8.87 (s, 1H), 7.42-7.20 (m, 14H), 6.52 (s, 1H), 6.07 (d, 1H), 5.02 (s, 2H), 4.92 (s, 2H), 4.03 (t, 2H), 3.50 (m, 2H), 3.27 (m, 1H), 1.85 (q, 2H), 1.27 (t, 3H), 1.13 (d, 6H), 0.90 (t, 3H)

### Step 2

### 5-(2,4-Dihydroxy-5-isopropylphenyl)-N-ethyl-4-(1-propyl-1H-pyrazol-3-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (19.6 mg, 0.05 mmol) in a yield of 40%.

¹H-NMR (400 MHz, CD₃OD) δ 7.56 (d, 1H), 7.00 (s, 1H), 6.40 (s, 1H), 6.20 (d, 1H), 4.10 (t, 2H), 3.42 (q, 2H), 3.14 (m, 2H), 1.87 (m, 2H), 1.24 (t, 3H), 1.11 (d, 6H), 0.90 (t, 3H)

### Example 102

### 5-(2,4-Dihydroxy-5-isopropylphenyl)-N-ethyl-4-(1-methyl-1H-pyrazol-4-yl)isoxazole-3-carboxamide (I-102)

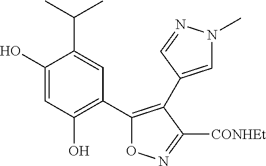

### Step 1

### 5-(2,4-Bis(benzyloxy)-5-isopropylphenyl)-N-ethyl-4-(1-meth 1-1H-pyrazol-4-isoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (100 mg, 0.17 mmol) was reacted with tetrakis(triphenylphosphine)palladium(0) (19.4 mg, 0.02 mmol) and 1-methyl-4-(tributylstannyl)-1H-pyrazole (66 μl, 0.20 mmol) to afford the intermediate compound 5-(2,4-bis(benzyloxy)-5-isopropylphenyl)-N-ethyl-4-(1-methyl-1H-pyrazol-4-yl)isoxazole-3-carboxamide (934 mg, 1.89 mmol) in a yield of 75%.

¹H-NMR (400 MHz, CDCl₃) δ 7.84 (s, 1H), 7.55-7.33 (m, 6H), 7.32-7.24 (m, 3H), 7.23 (s, 1H), 7.12 (dd, 2H), 6.84 (br t, 1H), 6.56 (s, 1H), 5.05 (s, 2H), 4.90 (s, 2H), 3.80 (s, 3H), 3.48 (m, 2H), 3.31 (sept, 1H), 1.26 (t, 3H), 1.18 (d, 6H)

### Step 2

### 5-(2,4-Dihydroxy-5-isopropylphenyl)-N-ethyl-4-(1-methyl-1H-pyrazol-4-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (23 mg, 0.06 mmol) in a yield of 37%.

¹H-NMR (400 MHz, CD₃OD) δ 7.68 (s, 1H), 7.39 (s, 1H), 6.97 (s, 1H), 6.41 (s, 1H), 3.83 (s, 3H), 3.41 (q, 2H), 3.16 (sept, 1H), 1.22 (t, 3H), 1.12 (d, 6H)

### Example 103

### 5-(2,4-Dihydroxy-5-isopropylphenyl)-N-ethyl-4-(1H-pyrazol-4-yl)isoxazole-3-carboxamide (I-103)

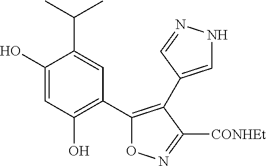

### Step 1

### 5-(2,4-Bis(benzyloxy)-5-isopropylphenyl)-N-ethyl-4-(1-trityl-1H-pyrazol-4-yl)isoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (60 mg, 0.10 mmol) was reacted with tetrakis(triphenylphosphine)palladium(0) (11.6 mg, 0.01 mmol) and 4-(tributylstannyl)-1-trityl-1H-pyrazole (72.3 mg, 0.25 mmol) to afford the crude intermediate compound 5-(2,4-bis(benzyloxy)-5-isopropylphenyl)-N-ethyl-4-(1-trityl-1H-pyrazol-4-yl)isoxazole-3-carboxamide.

¹H-NMR (400 MHz, CDCl₃) δ 7.73 (s, 1H), 7.63 (s, 1H), 7.42-7.30 (m, 10H), 7.25-7.18 (m, 8H), 7.16-7.06 (m, 8H), 6.71 (br t, 1H), 6.46 (s, 1H), 5.03 (s, 2H), 4.86 (s, 2H), 3.45 (m, 2H), 3.26 (sept, 1H), 1.22 (t, 3H), 1.12 (d, 6H)

### Step 2

### 5-(2,4-Bis(benzyloxy)-5-isopropylphenyl)-N-ethyl-4-(1H-pyrazol-4-yl)isoxazole-3-carboxamide

This intermediate compound (Step 1) was dissolved in methylene chloride/MeOH (1/2) (3.2 ml), trifluoroacetic acid (0.8 ml) was added. This reaction mixture was stirred at 75° C. for 3 h, and cooled to ambient temperature. Solvents were evaporated in vacuo, and the residue was purified by silica gel column chromatography to afford the intermediate compound 5-(2,4-bis(benzyloxy)-5-isopropylphenyl)-N-ethyl-4-(1H-pyrazol-4-yl)isoxazole-3-carboxamide (47 mg, 0.09 mmol) in a yield of 87%.

¹H-NMR (400 MHz, CDCl₃) δ 7.68 (br m, 2H), 7.42-7.27 (m, 8H), 7.20 (s, 1H), 7.13 (dd, 2H), 6.59 (s, 1H), 5.07 (s, 2H), 4.91 (s, 2H), 3.46 (m, 2H), 3.30 (sept, 1H), 1.26 (t, 3H), 1.16 (d, 6H)

### Step 3

### 5-(2,4-Dihydroxy-5-isopropylphenyl)-N-ethyl-4-(1H-pyrazol-4-yl)isoxazole-3-carboxamide

This compound was made using the procedure described for example 1 (Step 3). Thus, this intermediate compound (Step 2) was reacted with BCl₃ to afford a crude product, which was purified by silica gel column chromatography to afford the title compound (17 mg, 0.05 mmol) in a yield of 57%.

¹H-NMR (400 MHz, CD₃OD) δ 7.72-7.45 (br m, 2H), 6.97 (s, 1H), 6.41 (s, 1H), 3.41 (q, 2H), 3.16 (sept, 1H), 1.22 (t, 3H), 1.11 (d, 6H)

### Example 104

### 5-(2,4-Dihydroxy-5-isopropylphenyl)-N-ethyl-4-(1-ethyl-1H-pyrazol-4-yl)isoxazole-3-carboxamide (I-104)

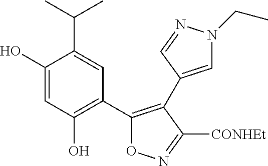

### Step 1

### 5-(2,4-Bis(benzyloxy)-5-isopropylphenyl)-N-ethyl-4-(1-ethyl-1H-pyrazol-4-yl)isoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (100 mg, 0.17 mmol) was reacted with tetrakis(triphenylphosphine)palladium(0) (19.4 mg, 0.02 mmol) and 4-(tributylstannyl)-1-ethyl-1H-pyrazole (77.5 mg, 0.20 mmol) to afford the crude intermediate compound 5-(2,4-bis(benzyloxy)-5-isopropylphenyl)-N-ethyl-4-(1-ethyl-1H-pyrazol-4-yl)isoxazole-3-carboxamide.

¹H-NMR (400 MHz, CDCl₃) δ 7.92 (s, 1H), 7.41-7.25 (m, 9H), 7.21 (s, 1H), 7.14 (dd, 2H), 6.84 (br t, 1H), 6.56 (s, 1H), 5.05 (s, 2H), 4.91 (s, 2H), 4.08 (q, 2H), 3.49 (m, 2H), 3.30 (sept, 1H), 1.41 (t, 3H), 1.26 (t, 3H), 1.17 (d, 6H)

### Step 2

### 5-(2,4-Dihydroxy-5-isopropylphenyl)-N-ethyl-4-(1-ethyl-1H-pyrazol-4-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (22 mg, 0.06 mmol) in a yield of 34%.

¹H-NMR (400 MHz, CD₃OD) δ 7.72 (s, 1H), 7.41 (s, 1H), 6.97 (s, 1H), 6.41 (s, 1H), 4.12 (q, 2H), 3.41 (q, 2H), 3.16 (sept, 1H), 1.41 (t, 3H), 1.23 (t, 3H), 1.11 (d, 6H)

### Example 105

### 5-(2,4-Dihydroxy-5-isopropylphenyl)-N-ethyl-4-(1-isopropyl-1H-pyrazol-4-yl)isoxazole-3-carboxamide (I-105)

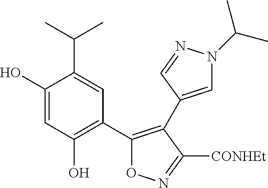

### Step 1

### 5-(2,4-Bis(benzyloxy)-5-isopropylphenyl)-N-ethyl-4-(1-isopropyl-1H-pyrazol-4-yl)isoxazole-3-carboxamide

This compound was made using the procedure described for example 7 (Step 1). Thus, 5-(2,4-bis(benzyloxy)-5-isopropylphenyl)-N-ethyl-4-iodoisoxazole-3-carboxamide (100 mg, 0.17 mmol) was reacted with tetrakis(triphenylphosphine)palladium(0) (19.4 mg, 0.02 mmol) and 4-(tributylstannyl)-1-isopropyl-1H-pyrazole (100.4 mg, 0.25 mmol) to afford the crude intermediate compound 5-(2,4-bis(benzyloxy)-5-isopropylphenyl)-N-ethyl-4-(1-isopropyl-1H-pyrazol-4-yl)isoxazole-3-carboxamide.

¹H-NMR (400 MHz, CDCl₃) δ 7.97 (s, 1H), 7.41-7.25 (m, 9H), 7.20 (s, 1H), 7.14 (dd, 2H), 6.84 (br t, 1H), 6.55 (s, 1H), 5.04 (s, 2H), 4.91 (s, 2H), 4.41 (sept, 1H), 3.49 (m, 2H), 3.30 (sept, 1H), 1.44 (d, 6H), 1.26 (t, 3H), 1.16 (d, 6H)

### Step 2

### 5-(2,4-Dihydroxy-5-isopropylphenyl)-N-ethyl-4-(1-isopropyl-1H-pyrazol-4-yl)isoxazole-3-carboxamide

This compound was made using the procedure described for example 1 (Step 3). Thus, this intermediate compound (Step 1) was reacted with BCl₃ to afford a crude product, which was purified by silica gel column chromatography to afford the title compound (38 mg, 0.09 mmol) in a yield of 57%.

¹H-NMR (400 MHz, CD₃OD) δ 7.74 (s, 1H), 7.41 (s, 1H), 6.96 (s, 1H), 6.42 (s, 1H), 4.46 (sept, 1H), 3.41 (q, 2H), 3.16 (sept, 1H), 1.44 (d, 6H), 1.23 (t, 3H), 1.11 (d, 6H)<

### Example 106

### Sodium 4-(3-(ethylcarbamoyl)-4-(5-methyl-1,2,4-oxadiazol-3-yl)isoxazol-5-yl)-6-isopropylbenzene-1,3-bis(olate) (I-106)

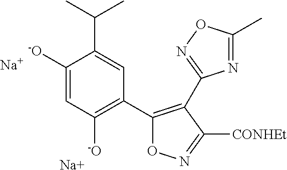

5-(2,4-Dihydroxy-5-isopropylphenyl)-N-ethyl-4-(5-methyl-1,2,4-oxadiazol-3-yl) isoxazole-3-carboxamide (92.6 mg, 0.249 mmol) was suspended in H₂O (4.6 ml). Sodium carbonate (52.7 mg, 0.497 mmol) was added. The reaction mixture was stirred at RT for 30 min, and filtered by solid impurities and the filtrate was freeze dried in vacuo to afford the title compound (100 mg, 0.24 mmol) in a yield of 96%.

### Example 107

### 4-(3-(Ethylcarbamoyl)-4-(5-methyl-1,2,4-oxadiazol-3-yl)isoxazol-5-yl)-6-isopropyl-1,3-phenylene diacetate (II-1)

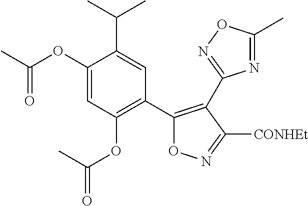

5-(2,4-Dihydroxy-5-isopropylphenyl)-N-ethyl-4-(5-methyl-1,2,4-oxadiazol-3-yl) isoxazole-3-carboxamide (150 mg, 0.40 mmol) was dissolved in THF (5 ml). Triethylamine (140 μl, 1.00 mmol), acetic anhydride (114 μl, 1.21 mmol) and 4-(dimethylamino)pyridine (4.9 mg, 0.04 mmol) were added sequentially. The reaction mixture was stirred at RT for overnight. Ethyl acetate was added to the solution. The organic phase was washed with 2N—HCl, saturated aqueous NaCl, dried with magnesium sulfate, and evaporated in vacuo to afford the title compound (179 mg, 0.39 mmol) in a yield of 97%.

¹H-NMR (400 MHz, CDCl₃) δ 7.65 (s, 1H), 6.98 (s, 1H), 6.91 (br t, 1H), 3.49 (m, 2H), 3.05 (sept, 1H), 2.63 (s, 3H), 2.33 (s, 3H), 2.17 (s, 3H), 1.26 (t, 3H), 1.20 (d, 6H)

### Example 108

### 4-(3-(Ethylcarbamoyl)-4-(5-methyl-1,2,4-oxadiazol-3-yl)isoxazol-5-yl)-6-isopropyl-1,3-phenylene dibutyrate (II-2)

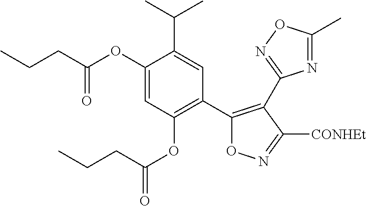

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (150 mg, 0.40 mmol) was reacted with triethylamine (140 μl, 1.00 mmol), butyryl chloride (125.5 μl, 1.21 mmol) and 4-(dimethylamino)pyridine (4.9 mg, 0.04 mmol) to afford the title compound (206 mg, 0.40 mmol) in a yield of 99%.

¹H-NMR (400 MHz, CDCl₃) δ 7.64 (s, 1H), 6.97 (s, 1H), 6.91 (br t, 1H), 3.49 (m, 2H), 3.03 (sept, 1H), 2.62 (s, 3H), 2.57 (t, 2H), 2.42 (dd, 2H), 1.80 (sext, 2H), 1.66 (sext, 2H), 1.26 (t, 3H), 1.19 (d, 6H), 1.05 (t, 3H), 0.97 (t, 3H)

### Example 109

### 5-(4-(3-(Ethylcarbamoyl)-4-(5-methyl-1,2,4-oxadiazol-3-yl)isoxazol-5-yl)-5-hydroxy-2-isopropylphenoxy)-5-oxopentanoic acid (II-3)

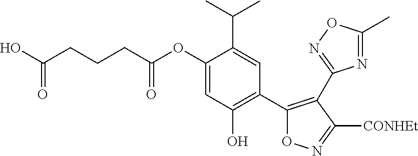

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (70 mg, 0.19 mmol) was reacted with glutaric anhydride (64 mg, 0.56 mmol) and 4-(dimethylamino)pyridine (2.3 mg, 0.02 mmol) to afford the title compound (55 mg, 0.11 mmol) in a yield of 60%.

¹H-NMR (400 MHz, CDCl₃) δ 7.39 (s, 1H), 7.14 (br s, 1H), 6.63 (s, 1H), 3.43 (m, 2H), 2.86 (sept, 1H), 2.55 (s, 3H), 2.31 (br t, 2H), 2.13 (br t, 2H), 1.94 (br t, 2H), 1.21 (t, 3H), 1.10 (d, 6H)

### Example 110

### (2S,2′S)-1-tert-Butyl^{′2},2-4-(3-(ethylcarbamoyl)-4-(5-methyl-1,2,4-oxadiazol-3-yl)isoxazol-5-yl)-6-isopropyl-1,3-phenylene dipyrrolidine-1,2-dicarboxylate (II-4)

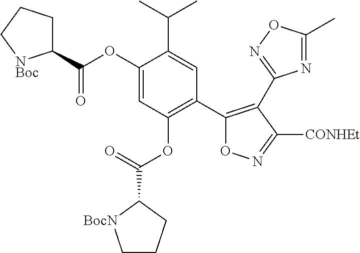

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (80 mg, 0.21 mmol) was reacted with Boc-Pro-OH (140 mg, 0.64 mmol), 4-(dimethylamino)pyridine (13 mg, 0.11 mmol) and 1-(3-dimethylaminopropyl)-3-ethylcarbodiimide hydrochloride (144 mg, 0.75 mmol) to afford the title compound (140 mg, 0.18 mmol) in a yield of 85%.

¹H-NMR (400 MHz, CDCl₃) δ 7.65 (m, 1H), 7.06-7.02 (m, 2H), 4.54 (m, 1H), 4.39 (m, 1H), 3.58-3.45 (m, 6H), 3.16-2.99 (m, 1H), 2.61 (s, 3H), 2.45-1.89 (m, 8H), 1.47-1.44 (m, 13H), 1.39-1.38 (m, 5H), 1.28-1.25 (m, 3H), 1.20-1.14 (m, 6H)

### Example 111

### (2S,2′S)-4-(3-(Ethylcarbamoyl)-4-(5-methyl-1,2,4-oxadiazol-3-yl)isoxazol-5-yl)-6-isopropyl-1,3-phenylene bis(2-(tert-butoxycarbonylamino)propanoate) (II-5)

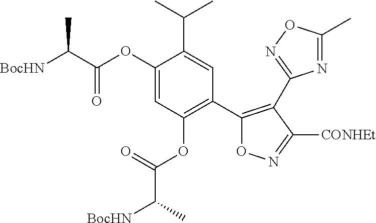

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (50 mg, 0.13 mmol) was reacted with Boc-Ala-OH (76.2 mg, 0.40 mmol), 4-(dimethylamino)pyridine (8.2 mg, 0.07 mmol) and 1-(3-dimethylaminopropyl)-3-ethylcarbodiimide hydrochloride (90 mg, 0.47 mmol) to afford the title compound (86 mg, 0.12 mmol) in a yield of 89%.

¹H-NMR (400 MHz, CDCl₃) δ 7.66 (s, 1H), 7.00 (br s, 2H), 5.04 (br t, 2H), 4.57 (br t, 1H), 4.43 (br t, 1H), 3.49 (m, 2H), 3.06 (sept, 1H), 2.63 (s, 3H), 1.57 (s, 6H), 1.46 (s, 9H), 1.43 (s, 9H), 1.26 (t, 3H), 1.19 (d, 6H)

### Example 112

### (2S,2′S)-4-(3-(Ethylcarbamoyl)-4-(5-methyl-1,2,4-oxadiazol-3-yl)isoxazol-5-yl)-6-isopropyl-1,3-phenylene bis(5-(2,3-bis(tert-butoxycarbonyl)guanidino)-2-(tert-butoxycarbonylamino)pentanoate) (II-6)

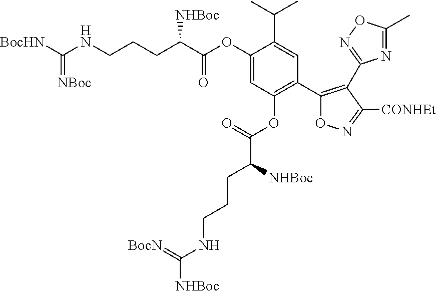

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (50 mg, 0.13 mmol) was reacted with Boc-Arg(Boc)₂-OH (191 mg, 0.40 mmol), 4-(dimethylamino)pyridine (8.2 mg, 0.07 mmol), and 1-(3-dimethylaminopropyl)-3-ethylcarbodiimide hydrochloride (90 mg, 0.47 mmol) to afford the title compound (153 mg, 0.12 mmol) in a yield of 88%.

¹H-NMR (400 MHz, CDCl₃) δ 9.39-9.15 (br m, 4H), 7.66 (s, 1H), 7.33 (br t, 1H), 6.99 (s, 1H), 5.79 (d, 1H), 5.64 (d, 1H), 4.49 (m, 1H), 4.39 (m, 1H), 4.01 (m, 2H), 3.85 (m, 2H), 3.48 (quint, 2H), 3.05 (sept, 1H), 2.62 (s, 3H), 2.00-1.69 (m, 8H), 1.53 (s, 9H), 1.51 (s, 9H), 1.48 (s, 9H), 1.47 (s, 9H), 1.45 (s, 9H), 1.42 (s, 9H), 1.26 (t, 3H), 1.16 (d, 6H)

### Example 113

### (2S,2′S)-4-(3-(Ethylcarbamoyl)-4-(5-methyl-1,2,4-oxadiazol-3-yl)isoxazol-5-yl)-6-isopropyl-1,3-phenylene bis(2-(tert-butoxycarbonylamino)-3-methylbutanoate) (II-7)

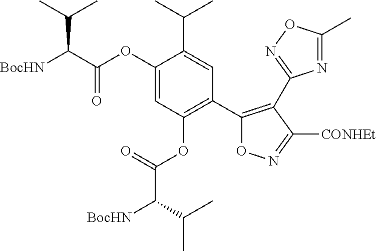

This compound was made using the procedure described for example 106. Thus, 5-(2,4-dihydroxy-5-isopropylphenyl)-N-ethyl-4-(5-methyl-1,2,4-oxadiazol-3-yl) isoxazole-3-carboxamide (50 mg, 0.13 mmol) was reacted with Boc-Val-OH (87.5 mg, 0.40 mmol), 4-(dimethylamino)pyridine (8.2 mg, 0.07 mmol), and 1-(3-dimethylaminopropyl)-3-ethylcarbodiimide hydrochloride (90 mg, 0.47 mmol) to afford the title compound (96 mg, 0.12 mmol) in a yield of 93%.

¹H-NMR (400 MHz, CDCl₃) δ 7.65 (s, 1H), 7.02-6.98 (m, 2H), 5.05 (dd, 2H), 4.47 (m, 1H), 4.34 (m, 1H), 3.49 (m, 2H), 3.08 (sept, 1H), 2.62 (s, 3H), 2.33 (m, 1H), 2.17 (m, 1H), 1.46 (s, 9H), 1.44 (s, 9H), 1.27 (t, 3H), 1.19 (d, 6H), 1.10 (d, 3H), 1.02 (d, 3H), 0.98 (d, 3H), 0.87 (d, 3H)

### Example 114

### (2S,2′S)-1-tert-Butyl^{′2},2-4-(3-(ethylcarbamoyl)-4-(1-methyl-1H-pyrazol-3-yl)isoxazol-5-yl)-6-isopropyl-1,3-phenylene dipyrrolidine-1,2-dicarboxylate (II-8)

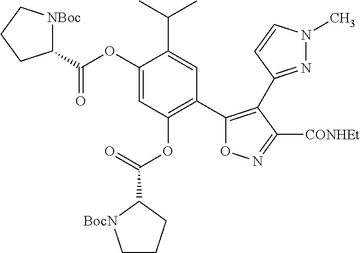

This compound was made using the procedure described for example 106. Thus, 5-(2,4-dihydroxy-5-isopropylphenyl)-N-ethyl-4-(1-methyl-1H-pyrazol-3-yl)isoxazole-3-carboxamide (49.7 mg, 0.13 mmol) was reacted with Boc-Pro-OH (86.7 mg, 0.40 mmol), 4-(dimethylamino)pyridine (8.2 mg, 0.07 mmol), and 1-(3-dimethylaminopropyl)-3-carbodiimide hydrochloride (90 mg, 0.46 mmol) to afford the title compound (102.6 mg, 0.13 mmol) in a yield of 99%.

¹H-NMR (400 MHz, CDCl₃) δ 8.30 (t, 1H), 7.47-7.30 (m, 2H), 7.09 (m, 1H), 6.23 (m, 1H), 4.56 (t, 1H), 4.36 (m, 1H), 3.91 (s, 3H), 3.65-3.30 (m, 6H), 2.96 (t, 1H), 2.48-1.82 (m, 6H), 1.49-1.37 (m, 18H), 1.26 (t, 3H), 1.13-1.05 (m, 6H)

### Example 115

### 5-(4-(3-(Ethylcarbamoyl)-4-(1-methyl-1H-pyrazol-3-yl)isoxazol-5-yl)-5-hydroxy-2-isopropylphenoxy)-5-oxopentanoic acid (II-9)

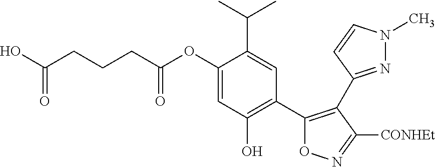

This compound was made using the procedure described for example 106. Thus, 5-(2,4-dihydroxy-5-isopropylphenyl)-N-ethyl-4-(1-methyl-1H-pyrazol-3-yl)isoxazole-3-carboxamide (50 mg, 0.14 mmol) was reacted with glutaric anhydride (46.2 mg, 0.41 mmol) and 4-(dimethylamino)pyridine (1.1 mg, 0.01 mmol) to afford the title compound (37 mg, 0.08 mmol) in a yield of 57%.

¹H-NMR (400 MHz, CDCl₃) δ 7.54 (s, 1H), 7.26 (s, 1H), 6.62 (s, 1H), 6.21 (s, 1H), 3.89 (s, 3H), 3.39 (m, 2H), 2.71 (m, 1H), 2.45 (m, 2H), 2.33 (m, 6H), 2.

### Experiment 1

### In Vitro Test for Antitumoral Activity

For present invention, the ATPase activity of yeast HSP90 was measured using malachite green assay. Formula I compounds for primary antitumoral activity were treated in human colorectal cancer cells (HCT116) showing increased expression levels of HSP90 client protein and cotoxicity IC₅₀ values was measured as a result.

TABLE 1
In vitro test for antitumoral activity.

IC₅₀ (nM)

| | Hsp90 | Growth |
|---|---|---|
| | ATPase | Inhibition |
| Compound | Assay | Assay |
| I-1 | A | A |
| I-2 | C | A |
| I-3 | C | C |
| I-4 | C | C |
| I-5 | C | A |
| I-6 | C | A |
| I-7 | B | B |
| I-8 | B | A |
| I-9 | B | A |
| I-10 | B | C |
| I-11 | A | A |
| I-12 | A | A |
| I-13 | A | A |
| I-14 | A | A |
| I-15 | A | C |
| I-16 | A | A |
| I-17 | A | A |
| I-18 | B | A |
| I-19 | A | A |
| I-20 | A | A |
| I-21 | B | A |
| I-22 | B | A |
| I-23 | B | A |
| I-24 | B | C |
| I-25 | B | C |
| I-26 | C | C |
| I-27 | A | A |
| I-28 | A | A |
| I-29 | C | C |
| I-30 | B | A |
| I-31 | B | A |
| I-32 | A | A |
| I-33 | C | C |
| I-34 | A | A |
| I-35 | A | A |
| I-36 | A | A |
| I-37 | A | A |
| I-38 | B | A |
| I-39 | B | A |
| I-40 | B | A |
| I-41 | A | A |
| I-42 | A | A |
| I-43 | B | A |
| I-44 | A | A |
| I-45 | A | A |
| I-46 | B | A |
| I-47 | A | A |
| I-48 | B | A |
| I-49 | B | B |
| I-50 | B | A |
| I-51 | A | A |
| I-52 | A | A |
| I-53 | B | A |
| I-54 | B | A |
| I-55 | B | A |
| I-56 | A | A |
| I-57 | B | A |
| I-58 | B | C |
| I-59 | B | A |
| I-60 | A | A |
| I-61 | A | A |
| I-62 | A | A |
| I-63 | A | A |
| I-64 | A | A |
| I-65 | A | A |
| I-66 | B | A |
| I-67 | B | B |
| I-68 | B | A |
| I-69 | B | A |
| I-70 | B | B |
| I-71 | A | A |
| I-72 | A | A |
| I-73 | B | A |
| I-74 | B | B |
| I-75 | A | A |
| I-76 | A | A |
| I-77 | B | A |
| I-78 | A | A |
| I-79 | B | A |
| I-80 | C | C |
| I-81 | B | A |
| I-82 | B | B |
| I-83 | B | A |
| I-84 | B | B |
| I-85 | A | A |
| I-86 | B | A |
| I-87 | A | B |
| I-88 | A | A |
| I-89 | B | A |
| I-90 | B | C |
| I-91 | B | A |
| I-92 | B | A |
| I-93 | B | A |
| I-94 | A | A |
| I-95 | B | A |
| I-96 | B | A |
| I-97 | B | A |
| I-98 | A | A |
| I-99 | B | A |
| I-100 | B | A |
| I-101 | B | A |
| I-102 | A | A |
| I-103 | A | A |
| I-104 | B | A |
| I-105 | B | A |

IC₅₀ results were allocated to one of 3 ranges as follows:
Range A: IC₅₀ < 500 nM
Range B: IC₅₀ 500 nM~IC₅₀ 1000 nM
Range C: IC₅₀ > 1000 nM

As can be seen from Table 1, Most of the compounds of the present invention exhibited significant antitumor activity for ATPase inhibitory activity and Growth inhibition assay.

### Experiment 2

### In Vivo Test for Antitumoral Activity

To evaluate the in vivo antitumor efficacy, the invented compound (example 60) was determined as follows.

A2780 Human ovarian cancer cells were established as subcutaneous xenografts by injection of 8×10⁶ cells into the flanks of adult female Balb/c nude mice. Mice with established tumors (50-200 mm³) were selected for study (n=3-6/treatment group). The test compounds were formulated in ethanol-PEG400-DW and administered orally (po) at a dose of 200 mg/kg. The vehicle alone was administered to control groups. Animals were dosed 5 days per week (Monday through Friday) for 2 consecutive weeks. Animals were weighed and tumor size was determined twice weekly by caliper measurements, and tumor volumes were calculated (volume=[l×w²]/2(mm³), where l and w refer to the larger and smaller dimensions collected at each measurement). The mean tumor volumes of each group were calculated. The change in mean treated tumor volume was divided by the change in mean control tumor volume, multiplied by 100 and subtracted from 100% to give the tumor growth inhibition for each group.

TABLE 2

| Compound | Dose (mg/kg) | Schedule | % TGI |
|---|---|---|---|
| example 60 | 200 | po qdx5 | 50 |
| (I -60) | | | |

Tumor growth inhibition of the selected compounds is set out in the Table 2. The selected compounds showed significant tumor growth inhibition. Those compounds were well-tolerated, exhibiting no treatment-related toxicity at these doses.

According to Experiment 1 and 2, the novel compound of Formula I can be useful for antitumor agent.

### Experiment 3

### Mouse Pharmacokinetics

Pharmacokinetics test was carried out in order to confirm the change from invented prodrugs to parent compounds in vivo. Balb/c male mice are dosed with a prodrug by oral gavages. A single administration of a prodrug is given and a dose volume of 10 mL/kg is used for PO doses.

Blood samples are collected in lithium heparin coated tubes at 10, 20, 30, 60, 120 min. Plasma is isolated by centrifugation and frozen before analysis. Plasma samples are prepared by liquid-liquid extraction with Ethyl acetate containing internal standard. Quantification is by using a LC-MS/MS method specific to the selected compound. Animals that had been dosed the selected compound were analyzed for prodrug and the parent compound. Pharmacokinetics parameters are calculated using WinNonLinnon compartmental analysis software.

TABLE 3

AUC_{0-120 min}

| | Dose level | II-8 | 1-97 (parent |
|---|---|---|---|
| compound | (mg/kg) | (prodrug) | compound) |
| II-8 | 100 | 55187 | 354559 |

The plasma AUC_{0-120 min} after PO administration of a prodrug is set out in the Table 3. For example 113 (II-8), plasma AUC_{0-120 min} was determined following a single dose of 100 mg/kg. Accordingly, exmaple 113 (II-8) afforded optimal exposure of the active pharmaceutical agent (example 97, I-97) relative to the starting dose.

Therefore, the compounds of Formula II which are the prodrug of Formula I can be useful for antitumor agent.

1-25. (canceled)

26. A compound of Formula I, a tautomer, or a pharmaceutically acceptable salt thereof: 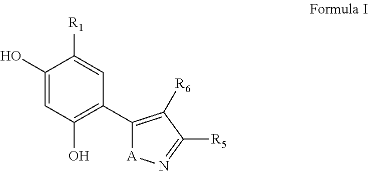
wherein:
A is a nitrogen atom or oxygen atom;
R₁ is chloro or isopropyl;
R₅ is CH₂R_{d} or N-ethylcarboxamide, wherein R_{d} is hydroxy, acetamido, propionamido or triazolyl; and
R₆ is 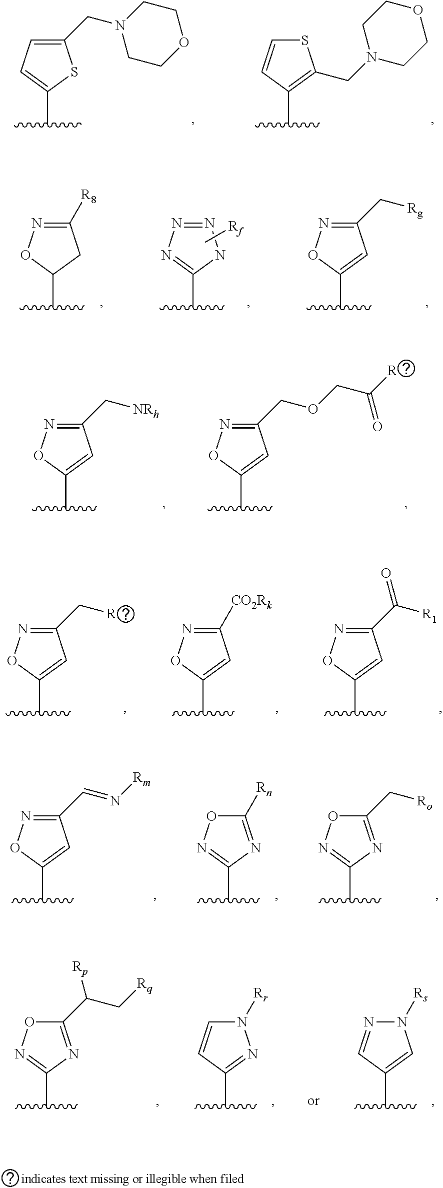
wherein Rₑ is hydroxymethyl, ethylcarboxylate or N-ethylcarboxamide; R_{f} is hydrogen, methyl or ethyl; R_{g} is hydrogen, hydroxy, fluoro, cyano, ethylamino, hydroxyethylamino, dimethylamino, diethylamino, isopropylamino, allylamino, diisopropylamino, piperidinyl, pyrrolidinyl, 4-methylpiperazinyl, morpholino, thiomorpholino or methanesulfonyl; Rₕ is hydrogen, acetyl or propionyl; Rᵢ is hydroxy, methoxy or amino; Rⱼ is cyano, thiophenyl, phenyl or dimethoxymethyl; Rₖ is hydrogen or ethyl; Rₗ is amino, methylamino, ethylamino, morpholino or thiomorpholino; Rₘ is hydroxy, methoxy, ethoxy or allyloxy; Rₙ is hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl; Rₒ is hydroxy, morpholino, dimethylamino, piperidinyl or pyrrolidinyl; Rₚ is (S)-hydroxy or hydroxy; R_{q} is hydrogen or chloro; Rᵣ is hydrogen, methyl, ethyl, isopropyl or n-propyl; Rₛ is hydrogen, methyl, ethyl, or isopropyl.

27. The compound according to claim 26, wherein
A is oxygen,
R₁ is isopropyl,
R₅ is N-ethylcarboxamide, and
R₆ is 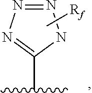
wherein R_{f} is hydrogen, methyl or ethyl.

28. The compound according to claim 26, wherein
A is oxygen,
R₁ is chloro or isopropyl,
R₅ is N-ethylcarboxamide, and
R₆ is 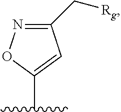
wherein R_{g} is hydrogen, hydroxy, fluoro, cyano, ethylamino, hydroxyethylamino, dimethylamino, diethylamino, isopropylamino, allylamino, diisopropylamino, piperidinyl, pyrrolidinyl, 4-methylpiperazinyl, morpholino, thiomorpholino or methanesulfonyl.

29. The compound according to claim 26, wherein
A is oxygen,
R₁ is chloro or isopropyl,
R₅ is N-ethylcarboxamide, and
R₆ is 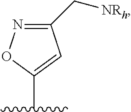
wherein Rₕ is hydrogen, acetyl or propionyl.

30. The compound according to claim 26, wherein
A is nitrogen or oxygen,
R₁ is isopropyl,
R₅ is N-ethylcarboxamide, and
R₆ is 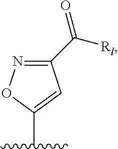
wherein R₁ is amino, methylamino, ethylamino, morpholino or thiomorpholino.

31. The compound according to claim 26, wherein
A is oxygen,
R₁ is isopropyl,
R₅ is N-ethylcarboxamide, and
R₆ is 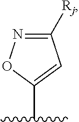
wherein Rⱼ is dimethoxymethyl.

32. The compound according to claim 26, wherein A is oxygen, R₁ is isopropyl, R₅ is N-ethylcarboxamide,
R₆ is 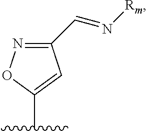
wherein Rₘ is hydroxy, methoxy, ethoxy or allyoxy.

33. The compound according to claim 26, wherein
A is oxygen,
R₁ is isopropyl,
R₅ is N-ethylcarboxamide, and
R₆ is 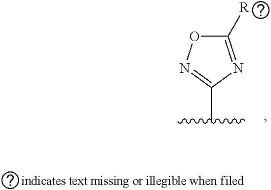
wherein Rₙ is hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl.

34. The compound according to claim 26, wherein
A is oxygen,
R₁ is isopropyl,
R₅ is N-ethylcarboxamide, and
R₆ is 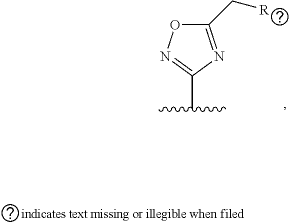
wherein Rₒ is hydroxy, morpholino, dimethylamino, piperidinyl or pyrrolidinyl.

35. The compound according to claim 26, wherein
A is oxygen,
R₁ is isopropyl,
R₅ is N-ethylcarboxamide, and
R₆ is 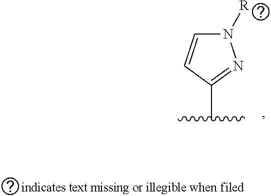
wherein Rᵣ is hydrogen, methyl, ethyl, isopropyl or n-propyl.

36. The compound according to claim 26, wherein
A is oxygen,
R₁ is isopropyl,
R₅ is N-ethylcarboxamide, and
R₆ is 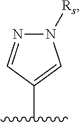
wherein Rₛ is hydrogen, methyl, ethyl, or isopropyl.

37. The compound according to claim 26, which is selected from the group consisting of:
5-(2,4-Dihydroxy-5-isopropylphenyl)-N-ethyl-4-(5-(morpholinomethyl)thiophen-2-yl)isoxazole-3-carboxamide;
5-(2,4-Dihydroxy-5-isopropylphenyl)-N-ethyl-4-(2-(morpholinomethyl)thiophen-3-yl)isoxazole-3-carboxamide;
4-(3-(Hydroxymethyl)-4-(thiophen-3-yl)isoxazol-5-yl)-6-isopropylbenzene-1,3-diol;
N-((5-(2,4-Dihydroxy-5-isopropylphenyl)-4-(thiophen-3-yl)isoxazol-3-yl)methyl)propionamide;
4-(3-((1H-1,2,3-Triazol-1-yl)methyl)-4-(thiophen-3-yl)isoxazol-5-yl)-6-isopropylbenzene-1,3-diol;
N-((5-(2,4-Dihydroxy-5-isopropylphenyl)-4-(thiophen-3-yl)isoxazol-3-yl)methyl)acetamide;
Ethyl 5-(5-(2,4-dihydroxy-5-isopropylphenyl)-3-(ethylcarbamoyl)isoxazol-4-yl)-4,5-dihydroisoxazole-3-carboxylate;
5-(2,4-Dihydroxy-5-isopropylphenyl)-N-ethyl-4-(3-(ethylcarbamoyl)-4,5-dihydroisoxazol-5-yl)isoxazole-3-carboxamide;
5-(2,4-Dihydroxy-5-isopropylphenyl)-N-ethyl-4-(3-(hydroxymethyl)-4,5-dihydroisoxazol-5-yl)isoxazole-3-carboxamide;
5-(2,4-Dihydroxy-5-isopropylphenyl)-N-ethyl-4-(1H-tetrazol-5-yl)isoxazole-3-carboxamide;
5-(2,4-Dihydroxy-5-isopropylphenyl)-N-ethyl-4-(1-methyl-1H-tetrazol-5-yl)isoxazole-3-carboxamide;
5-(2,4-Dihydroxy-5-isopropylphenyl)-N-ethyl-4-(2-methyl-2H-tetrazol-5-yl)isoxazole-3-carboxamide;
5-(2,4-Dihydroxy-5-isopropylphenyl)-N-ethyl-4-(1-ethyl-1H-tetrazol-5-yl)isoxazole-3-carboxamide;
5-(2,4-Dihydroxy-5-isopropylphenyl)-N-ethyl-4-(2-ethyl-2H-tetrazol-5-yl)isoxazole-3-carboxamide;
Ethyl 5-(2,4-dihydroxy-5-isopropylphenyl)-3-(ethylcarbamoyl)-4,5′-biisoxazole-3′-carboxylate;
5-(2,4-Dihydroxy-5-isopropylphenyl)-N³,N^{3′}-diethyl-4,5′-biisoxazole-3,3′-dicarboxamide;
5-(2,4-Dihydroxy-5-isopropylphenyl)-N-ethyl-3′-(hydroxymethyl)-4,5′-biisoxazole-3-carboxamide;
5-(2,4-Dihydroxy-5-isopropylphenyl)-N-ethyl-3′-(morpholinomethyl)-4,5′-biisoxazole-3-carboxamide;
Methyl 2-((5-(2,4-dihydroxy-5-isopropylphenyl)-3-(ethylcarbamoyl)-4,5′-biisoxazol-3′-yl)methoxy)acetate;
3′-((Diethylamino)methyl)-5-(2,4-dihydroxy-5-isopropylphenyl)-N-ethyl-4,5′-biisoxazole-3-carboxamide;
5-(2,4-Dihydroxy-5-isopropylphenyl)-N³-ethyl-4,5′-biisoxazole-3,3′-dicarboxamide;
5-(2,4-Dihydroxy-5-isopropylphenyl)-N-ethyl-3′-((2-hydroxyethylamino)methyl)-4,5′-biisoxazole-3-carboxamide;
5-(2,4-Dihydroxy-5-isopropylphenyl)-N-ethyl-3′-(morpholine-4-carbonyl)-4,5′-biisoxazole-3-carboxamide;
5-(5-Chloro-2,4-dihydroxyphenyl)-N-ethyl-3′-(hydroxymethyl)-4,5′-biisoxazole-3-carboxamide;
Ethyl 5-(5-chloro-2,4-dihydroxyphenyl)-3-(ethylcarbamoyl)-4,5′-biisoxazole-3′-carboxylate;
5-(2,4-Dihydroxy-5-isopropylphenyl)-3-(ethylcarbamoyl)-4,5′-biisoxazole-3′-carboxylic acid;
5-(2,4-Dihydroxy-5-isopropylphenyl)-N-ethyl-3′-((ethylamino)methyl)-4,5′-biisoxazole-3-carboxamide;
5-(2,4-Dihydroxy-5-isopropylphenyl)-N-ethyl-3′-(fluoromethyl)-4,5′-biisoxazole-3-carboxamide;
5-(5-(2,4-Dihydroxy-5-isopropylphenyl)-3-(ethylcarbamoyl)-1H-pyrazol-4-yl)-N-ethylisoxazole-3-carboxamide;
3′-(Aminomethyl)-5-(2,4-dihydroxy-5-isopropylphenyl)-N-ethyl-4,5′-biisoxazole-3-carboxamide;
3′-(Acetamidomethyl)-5-(2,4-dihydroxy-5-isopropylphenyl)-N-ethyl-4,5′-biisoxazole-3-carboxamide;
(5-(2,4-Dihydroxy-5-isopropylphenyl)-3-(ethylcarbamoyl)-4,5′-biisoxazol-3′-yl)methyl methanesulfonate;
2-((5-(2,4-Dihydroxy-5-isopropylphenyl)-3-(ethylcarbamoyl)-4,5′-biisoxazol-3′-yl)methoxy)acetic acid
5-(2,4-Dihydroxy-5-isopropylphenyl)-N-ethyl-3′-(propionamidomethyl)-4,5′-biisoxazole-3-carboxamide;
3′-(Cyanomethyl)-5-(2,4-dihydroxy-5-isopropylphenyl)-N-ethyl-4,5′-biisoxazole-3-carboxamide;
3′-((2-Amino-2-oxoethoxy)methyl)-5-(2,4-dihydroxy-5-isopropylphenyl)-N-ethyl-4,5′-biisoxazole-3-carboxamide;
5-(2,4-Dihydroxy-5-isopropylphenyl)-N-ethyl-3′-methyl-4,5′-biisoxazole-3-carboxamide;
5-(2,4-Dihydroxy-5-isopropylphenyl)-N-ethyl-3′-(piperidin-1-ylmethyl)-4,5′-biisoxazole-3-carboxamide;
5-(2,4-Dihydroxy-5-isopropylphenyl)-N-ethyl-3′-(pyrrolidin-1-ylmethyl)-4,5′-biisoxazole-3-carboxamide;
5-(2,4-Dihydroxy-5-isopropylphenyl)-N-ethyl-3′-((isopropylamino)methyl)-4,5′-biisoxazole-3-carboxamide;
5-(2,4-Dihydroxy-5-isopropylphenyl)-3′-((dimethylamino)methyl)-N-ethyl-4,5′-biisoxazole-3-carboxamide;
5-(2,4-Dihydroxy-5-isopropylphenyl)-N-ethyl-3′-((methylamino)methyl)-4,5′-biisoxazole-3-carboxamide;
5-(2,4-Dihydroxy-5-isopropylphenyl)-N-ethyl-3′-((4-methylpiperazin-1-yl)methyl)-4,5′-biisoxazole-3-carboxamide;
3′((Allylamino)methyl)-5-(2,4-dihydroxy-5-isopropylphenyl)-N-ethyl-4,5′-biisoxazole-3-carboxamide;
5-(2,4-Dihydroxy-5-isopropylphenyl)-3′-((dipropylamino)methyl)-N-ethyl-4,5′-biisoxazole-3-carboxamide;
5-(2,4-Dihydroxy-5-isopropylphenyl)-N-ethyl-3′-(thiophen-3-yl)-4,5′-biisoxazole-3-carboxamide;
5-(2,4-Dihydroxy-5-isopropylphenyl)-N-ethyl-3′-(thiomorpholinomethyl)-4,5′-biisoxazole-3-carboxamide;
3′-Cyano-5-(2,4-dihydroxy-5-isopropylphenyl)-N-ethyl-4,5′-biisoxazole-3-carboxamide;
5-(2,4-Dihydroxy-5-isopropylphenyl)-N-ethyl-3′-phenyl-4,5′-biisoxazole-3-carboxamide;
5-(2,4-Dihydroxy-5-isopropylphenyl)-N-ethyl-3′-(thiomorpholine-4-carbonyl)-4,5′-biisoxazole-3-carboxamide;
5-(2,4-Dihydroxy-5-isopropylphenyl)-3′-(dimethoxymethyl)-N-ethyl-4,5′-biisoxazole-3-carboxamide;
5-(2,4-Dihydroxy-5-isopropylphenyl)-N-ethyl-3′-((methoxyimino)methyl)-4,5′-biisoxazole-3-carboxamide;
5-(2,4-Dihydroxy-5-isopropylphenyl)-N-ethyl-3′-((hydroxyimino)methyl)-4,5′-biisoxazole-3-carboxamide;
3′-((Allyloxyimino)methyl)-5-(2,4-dihydroxy-5-isopropylphenyl)-N-ethyl-4,5′-biisoxazole-3-carboxamide;
5-(2,4-Dihydroxy-5-isopropylphenyl)-3′-((ethoxyimino)methyl)-N-ethyl-4,5′-biisoxazole-3-carboxamide;
5-(2,4-Dihydroxy-5-isopropylphenyl)-N³-ethyl-N^{3′}-methyl-4,5′-biisoxazole-3,3′-dicarboxamide;
5-(2,4-Dihydroxy-5-isopropylphenyl)-N-ethyl-4-(5-(trifluoromethyl)-1,2,4-oxadiazol-3-yl)isoxazole-3-carboxamide;
Methyl 3-(5-(2,4-dihydroxy-5-isopropylphenyl)-3-(ethylcarbamoyl)isoxazol-4-yl)-1,2,4-oxadiazole-5-carboxylate;
3-(5-(2,4-Dihydroxy-5-isopropylphenyl)-3-(ethylcarbamoyl)isoxazol-4-yl)-N-ethyl-1,2,4-oxadiazole-5-carboxamide;
5-(2,4-Dihydroxy-5-isopropylphenyl)-N-ethyl-4-(5-methyl-1,2,4-oxadiazol-3-yl)isoxazole-3-carboxamide;
5-(2,4-Dihydroxy-5-isopropylphenyl)-N-ethyl-4-(5-phenyl-1,2,4-oxadiazol-3-yl)isoxazole-3-carboxamide;
5-(2,4-Dihydroxy-5-isopropylphenyl)-N-ethyl-4-(5-ethyl-1,2,4-oxadiazol-3-yl)isoxazole-3-carboxamide;
5-(2,4-Dihydroxy-5-isopropylphenyl)-N-ethyl-4-(5-(furan-2-yl)-1,2,4-oxadiazol-3-yl)isoxazole-3-carboxamide;
3-(5-(2,4-Dihydroxy-5-isopropylphenyl)-3-(ethylcarbamoyl)isoxazol-4-yl)-N,N-dimethyl-1,2,4-oxadiazole-5-carboxamide;
5-(2,4-Dihydroxy-5-isopropylphenyl)-N-ethyl-4-(5-isopropyl-1,2,4-oxadiazol-3-yl)isoxazole-3-carboxamide;
5-(2,4-Dihydroxy-5-isopropylphenyl)-N-ethyl-4-(1,2,4-oxadiazol-3-yl)isoxazole-3-carboxamide;
5-(2,4-Dihydroxy-5-isopropylphenyl)-N-ethyl-4-(5-(morpholine-4-carbonyl)-1,2,4-oxadiazol-3-yl)isoxazole-3-carboxamide;
5-(2,4-Dihydroxy-5-isopropylphenyl)-N-ethyl-4-(5-(pyrrolidine-1-carbonyl)-1,2,4-oxadiazol-3-yl)isoxazole-3-carboxamide;
5-(2,4-Dihydroxy-5-isopropylphenyl)-N-ethyl-4-(5-(pyrrolidin-1-yl)-1,2,4-oxadiazol-3-yl)isoxazole-3-carboxamide;
5-(2,4-Dihydroxy-5-isopropylphenyl)-N-ethyl-4-(5-(trichloromethyl)-1,2,4-oxadiazol-3-yl)isoxazole-3-carboxamide;
5-(2,4-Dihydroxy-5-isopropylphenyl)-N-ethyl-4-(5-(piperidin-1-yl)-1,2,4-oxadiazol-3-yl)isoxazole-3-carboxamide;
5-(2,4-Dihydroxy-5-isopropylphenyl)-4-(5-(dimethylamino)-1,2,4-oxadiazol-3-yl)-N-ethylisoxazole-3-carboxamide;
5-(2,4-Dihydroxy-5-isopropylphenyl)-N-ethyl-4-(5-morpholino-1,2,4-oxadiazol-3-yl)isoxazole-3-carboxamide;
3-(5-(2,4-Dihydroxy-5-isopropylphenyl)-3-(ethylcarbamoyl)isoxazol-4-yl)-N,N-diethyl-1,2,4-oxadiazole-5-carboxamide;
5-(2,4-Dihydroxy-5-isopropylphenyl)-N-ethyl-4-(5-(methoxymethyl)-1,2,4-oxadiazol-3-yl)isoxazole-3-carboxamide;
4-(5-(Diethylamino)-1,2,4-oxadiazol-3-yl)-5-(2,4-dihydroxy-5-isopropylphenyl)-N-ethylisoxazole-3-carboxamide;
5-(2,4-Dihydroxy-5-isopropylphenyl)-N-ethyl-4-(5-(thiophen-2-yl)-1,2,4-oxadiazol-3-yl)isoxazole-3-carboxamide;
4-(5-Amino-1,2,4-oxadiazol-3-yl)-5-(2,4-dihydroxy-5-isopropylphenyl)-N-ethylisoxazole-3-carboxamide;
5-(2,4-Dihydroxy-5-isopropylphenyl)-N-ethyl-4-(5-(methylamino)-1,2,4-oxadiazol-3-yl)isoxazole-3-carboxamide;
5-(2,4-Dihydroxy-5-isopropylphenyl)-N-ethyl-4-(5-hydroxy-1,2,4-oxadiazol-3-yl)isoxazole-3-carboxamide;
5-(2,4-Dihydroxy-5-isopropylphenyl)-N-ethyl-4-(5-(hydroxymethyl)-1,2,4-oxadiazol-3-yl)isoxazole-3-carboxamide;
5-(2,4-Dihydroxy-5-isopropylphenyl)-N-ethyl-4-(5-mercapto-1,2,4-oxadiazol-3-yl)isoxazole-3-carboxamide;
(S)-5-(2,4-Dihydroxy-5-isopropylphenyl)-N-ethyl-4-(5-(1-hydroxyethyl)-1,2,4-oxadiazol-3-yl)isoxazole-3-carboxamide;
5-(2,4-Dihydroxy-5-isopropylphenyl)-N-ethyl-4-(5-(4-methoxyphenyl)-1,2,4-oxadiazol-3-yl)isoxazole-3-carboxamide;
5-(2,4-Dihydroxy-5-isopropylphenyl)-N-ethyl-4-(5-(4-nitrophenyl)-1,2,4-oxadiazol-3-yl)isoxazole-3-carboxamide;
5-(2,4-Dihydroxy-5-isopropylphenyl)-N-ethyl-4-(5-methoxy-1,2,4-oxadiazol-3-yl)isoxazole-3-carboxamide;
5-(2,4-Dihydroxy-5-isopropylphenyl)-N-ethyl-4-(5-(methylthio)-1,2,4-oxadiazol-3-yl)isoxazole-3-carboxamide;
4-(5-Cyclopentyl-1,2,4-oxadiazol-3-yl)-5-(2,4-dihydroxy-5-isopropylphenyl)-N-ethylisoxazole-3-carboxamide;
4-(5-Cyclohexyl-1,2,4-oxadiazol-3-yl)-5-(2,4-dihydroxy-5-isopropylphenyl)-N-ethylisoxazole-3-carboxamide;
5-(2,4-Dihydroxy-5-isopropylphenyl)-4-(5-(4-ethoxyphenyl)-1,2,4-oxadiazol-3-yl)-N-ethylisoxazole-3-carboxamide;
4-(5-(2-Chloro-1-hydroxyethyl)-1,2,4-oxadiazol-3-yl)-5-(2,4-dihydroxy-5-isopropylphenyl)-N-ethylisoxazole-3-carboxamide;
5-(2,4-Dihydroxy-5-isopropylphenyl)-N-ethyl-4-(5-(morpholinomethyl)-1,2,4-oxadiazol-3-yl)isoxazole-3-carboxamide;
5-(2,4-Dihydroxy-5-isopropylphenyl)-4-(5-((dimethylamino)methyl)-1,2,4-oxadiazol-3-yl)-N-ethylisoxazole-3-carboxamide;
5-(2,4-Dihydroxy-5-isopropylphenyl)-N-ethyl-4-(5-(piperidin-1-ylmethyl)-1,2,4-oxadiazol-3-yl)isoxazole-3-carboxamide;
5-(2,4-Dihydroxy-5-isopropylphenyl)-N-ethyl-4-(5-(pyrrolidin-1-ylmethyl)-1,2,4-oxadiazol-3-yl)isoxazole-3-carboxamide;
5-(2,4-Dihydroxy-5-isopropylphenyl)-N-ethyl-4-(1H-pyrazol-3-yl)isoxazole-3-carboxamide;
5-(2,4-Dihydroxy-5-isopropylphenyl)-N-ethyl-4-(1-methyl-1H-pyrazol-3-yl)isoxazole-3-carboxamide;
5-(2,4-Dihydroxy-5-isopropylphenyl)-N-ethyl-4-(1-methyl-1H-pyrazol-5-yl)isoxazole-3-carboxamide;
5-(2,4-Dihydroxy-5-isopropylphenyl)-N-ethyl-4-(1-ethyl-1H-pyrazol-3-yl)isoxazole-3-carboxamide;
5-(2,4-Dihydroxy-5-isopropylphenyl)-N-ethyl-4-(1-isopropyl-1H-pyrazol-3-yl)isoxazole-3-carboxamide;
5-(2,4-Dihydroxy-5-isopropylphenyl)-N-ethyl-4-(1-propyl-1H-pyrazol-3-yl)isoxazole-3-carboxamide;
5-(2,4-Dihydroxy-5-isopropylphenyl)-N-ethyl-4-(1-methyl-1H-pyrazol-4-yl)isoxazole-3-carboxamide;
5-(2,4-Dihydroxy-5-isopropylphenyl)-N-ethyl-4-(1H-pyrazol-4-yl)isoxazole-3-carboxamide;
5-(2,4-Dihydroxy-5-isopropylphenyl)-N-ethyl-4-(1-ethyl-1H-pyrazol-4-yl)isoxazole-3-carboxamide;
5-(2,4-Dihydroxy-5-isopropylphenyl)-N-ethyl-4-(1-isopropyl-1H-pyrazol-4-yl)isoxazole-3-carboxamide; and
Sodium 4-(3-(ethylcarbamoyl)-4-(5-methyl-1,2,4-oxadiazol-3-yl)isoxazol-5-yl)-6-isopropylbenzene-1,3-bis(olate),
or a tautomer or a pharmaceutically acceptable salt thereof.

38. A process for preparing a compound of Formula I, comprising:
subjecting a compound of Formula 2 to Suzuki-coupling or Stille cross-coupling with a substituted boronic acid or tributylstannane optionally in a solvent to prepare a compound of Formula 3 (Step 1);
subjecting the compound of Formula 3 to reduction, substitution, cyclization, reductive amination, hydrolysis, oxidation, dehydration, alcoholysis, or deacetylation to prepare a compound of Formula 4 (Step 2); and
subjecting the compound of Formula 4 to deprotection of benzyl group with BCl₃ to prepare a compound of Formula I (Step 3), 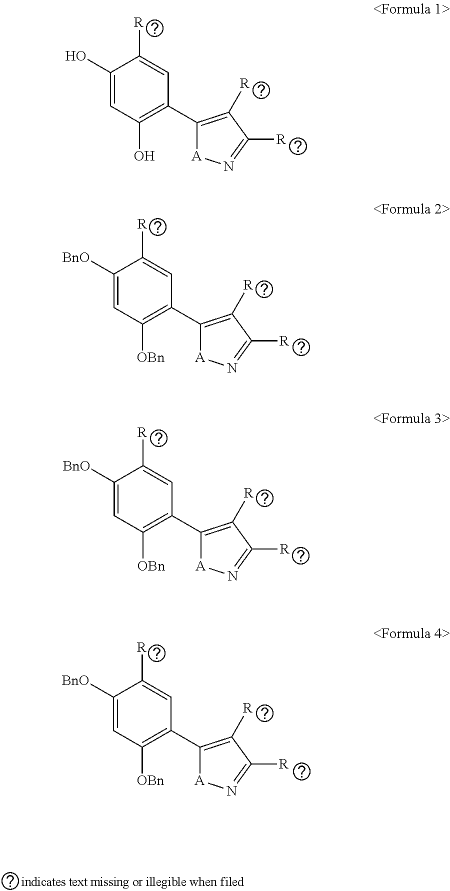
wherein:
A is a nitrogen atom or an oxygen atom,
R₁ is chloro or isopropyl;
R₂ is iodo;
R₃ is ethylcarboxylate or N-ethylcarboxamide,
R₄ is cyano, 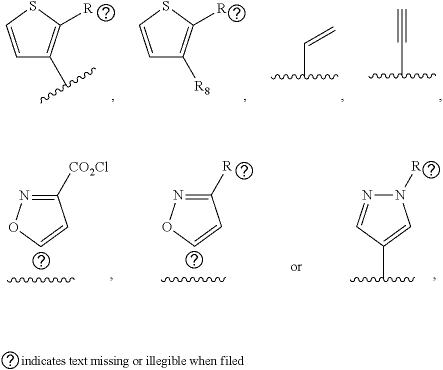
wherein Rₐ is hydrogen or formyl; R_{b} is methyl, thiophenyl or phenyl; R_{c} is hydrogen, trityl, methyl, ethyl or isopropyl;
R₅ is CH₂R_{d} or N-ethylcarboxamide, wherein R_{d} is hydroxyl, acetamido, propionamido or triazolyl;
R₆ is 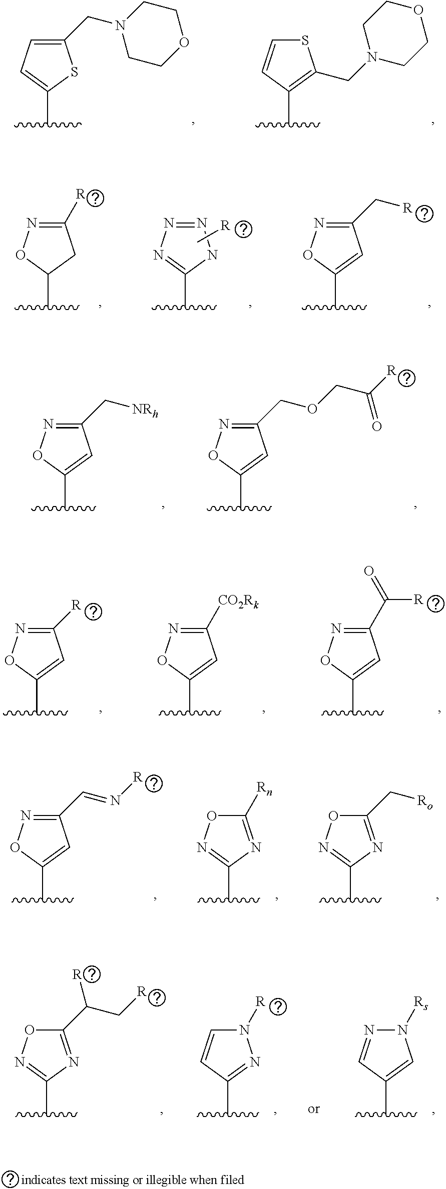
wherein
Rₑ is hydroxymethyl, ethylcarboxylate or N-ethylcarboxamide;
R_{f} is hydrogen, methyl or ethyl;
R_{g} is hydrogen, hydroxy, fluoro, cyano, ethylamino, hydroxyethyl amino, dimethylamino, diethylamino, isopropylamino, allylamino, diisopropylamino, piperidinyl, pyrrolidinyl, 4-methylpiperazinyl, morpholino, thiomorpholino or methanesulfonyl;
Rₕ is hydrogen, acetyl or propionyl;
Rᵢ is hydroxy, methoxy or amino;
Rⱼ is cyano, thiophenyl, phenyl or dimethoxymethyl;
Rₖ is hydrogen or ethyl;
Rₗ is amino, methylamino, ethylamino, morpholino or thiomorpholino;
Rₘ is hydroxy, methoxy, ethoxy or allyloxy;
Rₙ is hydrogen, methyl, ethyl, isopropyl, trifluoromethyl, methylcarboxylate, N-ethylcarboxamide, N,N-dimethylcarboxamide, 5-phenyl, 5-furanyl, morpholinocarbonyl, pyrrolidinocarbonyl, pyrrolidinyl, trichloromethyl, piperidinyl, dimethylamino, morpholino, N,N-diethylcarboxamide, diethylamino, methoxymethyl, 2-thiophenyl, amino, methylamino, hydroxy, mercapto, p-methoxyphenyl, p-nitrophenyl, methoxy, methylthio, cyclopentyl, cyclohexyl or p-ethoxyphenyl;
Rₒ is hydroxy, morpholino, dimethylamino, piperidinyl or pyrrolidinyl;
Rₚ is (S)-hydroxy or hydroxy;
R_{q} is hydrogen or chloro;
Rᵣ is hydrogen, methyl, ethyl, isopropyl or n-propyl; and
Rₛ is hydrogen, methyl, ethyl, or isopropyl.

39. A pharmaceutical composition comprising a therapeutically effective amount of a compound of Formula I according to claim 26, a tautomer, or a pharmaceutically acceptable salt thereof, and a pharmaceutically acceptable carrier.

40. A method of treating tumor comprising administering a therapeutically effective amount of a compound of Formula I according to claim 26, a tautomer, or a pharmaceutically acceptable salt thereof in a subject in need thereof.

41. A compound of Formula II as a prodrug of the compound of Formula I according to claim 26 or a pharmaceutically acceptable salt thereof, 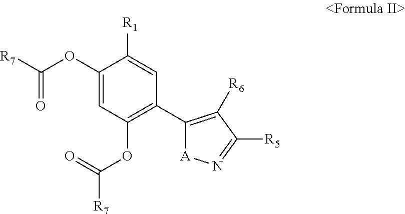
wherein:
A, R₁, R₅ and R₆ are as defined in claim 26 and
R₇ is acetyl, butyryl, 5-oxopentanoic acid, (tert-butoxycarbonyl)prolinyl, (tert-butoxycarbonyl)alaninyl,
5-(2,3-bis(tert-butoxycarbonyl)guanidino)-2-(tert-butoxycarbonyl)pentanoyl or (tert-butoxycarbonyl)valinyl.

42. The compound of Formula II according to claim 41, which is selected from the group consisting of:
4-(3-(Ethylcarbamoyl)-4-(5-methyl-1,2,4-oxadiazol-3-yl)isoxazol-5-yl)-6-isopropyl-1,3-phenylene diacetate;
4-(3-(Ethylcarbamoyl)-4-(5-methyl-1,2,4-oxadiazol-3-yl)isoxazol-5-yl)-6-isopropyl-1,3-phenylene dibutyrate;
5-(4-(3-(Ethylcarbamoyl)-4-(5-methyl-1,2,4-oxadiazol-3-yl)isoxazol-5-yl)-5-hydroxy-2-isopropylphenoxy)-5-oxopentanoic acid;
(2S,2′S)-1-tert-Butyl^{′2},2-4-(3-(ethylcarbamoyl)-4-(5-methyl-1,2,4-oxadiazol-3-yl)isoxazol-5-yl)-6-isopropyl-1,3-phenylene dipyrrolidine-1,2-dicarboxylate;
(2S,2′S)-4-(3-(Ethylcarbamoyl)-4-(5-methyl-1,2,4-oxadiazol-3-yl)isoxazol-5-yl)-6-isopropyl-1,3-phenylene bis(2-(tert-butoxycarbonylamino)propanoate);
(2S,2′S)-4-(3-(Ethylcarbamoyl)-4-(5-methyl-1,2,4-oxadiazol-3-yl)isoxazol-5-yl)-6-isopropyl-1,3-phenylene bis(5-(2,3-bis(tert-butoxycarbonyl)guanidino)-2-(tert-butoxycarbonylamino)pentanoate);
(2S,2′S)-4-(3-(Ethylcarbamoyl)-4-(5-methyl-1,2,4-oxadiazol-3-yl)isoxazol-5-yl)-6-isopropyl-1,3-phenylene bis(2-(tert-butoxycarbonylamino)-3-methylbutanoate);
(2S,2′S)-1-tert-Butyl^{′2},2-4-(3-(ethylcarbamoyl)-4-(1-methyl-1H-pyrazol-3-yl)isoxazol-5-yl)-6-isopropyl-1,3-phenylene dipyrrolidine-1,2-dicarboxylate; and
5-(4-(3-(Ethylcarbamoyl)-4-(1-methyl-1H-pyrazol-3-yl)isoxazol-5-yl)-5-hydroxy-2-isopropylphenoxy)-5-oxopentanoic acid,
or a pharmaceutically acceptable salt thereof.
